SEQUENCE LISTING (44 sequences)

SEQ ID NO: 1 - protein, 16 aa, Synthetic construct
YIHFSGYTNYNPSLKS

SEQ ID NO: 2 - protein, 16 aa, Synthetic construct
YIHFSGYTNYNGSLKS

SEQ ID NO: 3 - protein, 13 aa, Synthetic construct
YVHFSGYSNYSPS

SEQ ID NO: 4 - protein, 12 aa, Synthetic construct
YGHFSGYGNYGP

SEQ ID NO: 5 - protein, 12 aa, Synthetic construct
YGHFSPYGNYGP

SEQ ID NO: 6 - protein, 12 aa, Synthetic construct
YIHFSGYTNYNP

SEQ ID NO: 7 - protein, 12 aa, Synthetic construct
YIHFSPYTNYNP

SEQ ID NO: 8 - protein, 10 aa, Synthetic construct
YHFSYNYPKS

SEQ ID NO: 9 - protein, 8 aa, Synthetic construct
YHFSYNYP

SEQ ID NO: 10 - protein, 13 aa, Synthetic construct
VIDGSQSTDDDKI

SEQ ID NO: 11 - protein, 11 aa, Synthetic construct
DGSQSTDDDKI

SEQ ID NO: 12 - protein, 10 aa, Synthetic construct
DSQSTDDDKI

SEQ ID NO: 13 - protein, 9 aa, Synthetic construct
SGSTDDDKI

SEQ ID NO: 14 - protein, 19 aa, Synthetic construct
GYWIGPFTGGGYIHFSGYT

SEQ ID NO: 15 - protein, 19 aa, Synthetic construct
GYWIGPFTGPGYIHFSGYT

SEQ ID NO: 16 - protein, 19 aa, Synthetic construct
GYWIGPFTPGPYIHFSGYT

SEQ ID NO: 17 - protein, 20 aa, Synthetic construct
LITHPRDYSPKLTPGLYEFG

SEQ ID NO: 18 - protein, 8 aa, Synthetic construct
YIHFSGYT

SEQ ID NO: 19 - protein, 5 aa, Synthetic construct
STDDD

SEQ ID NO: 20 - protein, 5 aa, Synthetic construct
CYHFS

SEQ ID NO: 21 - protein, 9 aa, Synthetic construct
LITHPRDYS

SEQ ID NO: 22 - protein, 16 aa, Synthetic construct
GCGQQYWIGPFTFGCG

SEQ ID NO: 23 - protein, 12 aa, Synthetic construct
GQQYWIGPFTFG

SEQ ID NO: 24 - protein, 19 aa, Synthetic construct
LETVKPGLYEPITHPRDYS

SEQ ID NO: 25 - protein, 19 aa, Synthetic construct
SYDRPHTIPEYLGPKVTEL

SEQ ID NO: 26 - protein, 33 aa, Synthetic construct
AIVSPQFQEISLPTTSTVIDGSQSTDDDKIVQY

SEQ ID NO: 27 - protein, 11 aa, Synthetic construct
CSGSTEQEKIC

SEQ ID NO: 28 - protein, 18 aa, Synthetic construct
CVIDGSQSTDDDKIVQYC

SEQ ID NO: 29 - protein, 11 aa, Synthetic construct
GCLITHPRDYS

SEQ ID NO: 30 - protein, 14 aa, Synthetic construct
GCLITHPRDYSGCG

SEQ ID NO: 31 - protein, 17 aa, Synthetic construct
GYIHFSGYTNYNPSLKS

SEQ ID NO: 32 - protein, 17 aa, Synthetic construct
TVIDGSQSTDDDKIVQY

SEQ ID NO: 33 - protein, 13 aa, Synthetic construct
VIDGSASTDDDRI

SEQ ID NO: 34 - protein, 13 aa, Synthetic construct
VIDGSSSTDDDRI

SEQ ID NO: 35 - protein, 13 aa, Synthetic construct
VIDGSASTDDDHI

SEQ ID NO: 36 - DNA, 20 bp, Synthetic construct
  source 1..20: mol_type = other DNA
agcaaagaccccaacgagaa

SEQ ID NO: 37 - DNA, 15 bp, Synthetic construct
  source 1..15: mol_type = other DNA
ggcggcggtcacgaa

SEQ ID NO: 38 - DNA, 22 bp, Synthetic construct
  source 1..22: mol_type = other DNA
cgcgatcacatggtcctgctgg

SEQ ID NO: 39 - DNA, 26 bp, Synthetic construct
  source 1..26: mol_type = other DNA
tctgggacacaaattggaatacaact

SEQ ID NO: 40 - DNA, 25 bp, Synthetic construct
  source 1..25: mol_type = other DNA
cgggtcttgaagttcactttgattc

SEQ ID NO: 41 - DNA, 16 bp, Synthetic construct
  source 1..16: mol_type = other DNA
catggcagacaaacaa

SEQ ID NO: 42 - DNA, 22 bp, Synthetic construct
  source 1..22: mol_type = other DNA
agtaacgccaatagggactttc

SEQ ID NO: 43 - DNA, 22 bp, Synthetic construct
  source 1..22: mol_type = other DNA
ggcgtacttggcatatgataca

SEQ ID NO: 44 - DNA, 18 bp, Synthetic construct
  source 1..18: mol_type = other DNA
cggtaagcccacttggca

(19) United States
(12) Patent Application Publication
(10) Pub. No.: US 2026/0317783 A1
(43) Pub. Date: Oct. 15, 2026

(54) COMPOSITIONS AND METHODS FOR PURIFYING VIRAL VECTORS

(71) Applicant: North Carolina State University, Raleigh, NC (US)

(72) Inventors: Stefano Menegatti, Raleigh, NC (US); Shriarjun Shastry, Raleigh, NC (US); Wenning Chu, Raleigh, NC (US)

(21) Appl. No.: 19/483,663

(22) PCT Filed: May 13, 2024
(86) PCT No.: PCT/US2024/029159

§ 371 Date: Nov. 12, 2025

Related U.S. Application Data
(60) Provisional application No. 63/501,785, filed on May 12, 2023.

(51) Int. Cl. C12N 15/86; B01D 15/38; C07K 7/64; C07K 17/00; C12N 7/00

(52) U.S. Cl. CPC C12N 15/86; B01D 15/3804; C07K 7/64; C07K 17/00; C12N 7/00; C12N 2750/14143; C12N 2750/14151

(57) ABSTRACT

The present disclosure provides materials and methods related to the purification of viral vectors. In particular, the present disclosure provides compositions, and related methods, comprising peptide ligands capable of removing process-related impurities (e.g., host cell proteins, nucleic acids, and media components) and product-related impurities (e.g., product fragments, product aggregates, and inactive forms derived from product degradation by or association with other species in the cell culture harvest) from biological fluids during the production and purification of adeno-associated viruses (AAVs).

### CROSS REFERENCE TO RELATED APPLICATIONS

This application claims priority to and the benefit of U.S. Provisional Patent Application No. 63/501,785 filed May 12, 2023, which is incorporated herein by reference in its entirety for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 39,375 bytes Byte ASCII (Text) file named “NCSU-41892-601” created on May 13, 2024.

### FIELD

The present disclosure provides materials and methods related to the purification of viral vectors. In particular, the present disclosure provides compositions, and related methods, comprising peptide ligands capable of removing process-related impurities and product-related impurities from biological fluids during the production and purification of adeno-associated viruses (AAVs).

### BACKGROUND

Viral vectors are poised to become fundamental tools in modern medicine and biotechnology owing to their role as delivery agents of gene therapies targeting rare diseases, oncolytic agents to fight aggressive forms of cancer, vaccine platforms to counter infectious diseases, and a gateway to engineer plants and animals for a sustainable agriculture. The landscape of viral vector technology is rich of promises as much as challenges: novel vector designs are constantly being introduced with improved tissue targeting and gene delivery activity as well as lower genotoxicity, hepatotoxicity, and immunogenicity. At the same time, the bioprocess technology utilized in viral vector manufacturing draws heavily upon a decades-old platform established decades ago for producing monoclonal antibodies (mAbs). This is well exemplified by the purification pipeline of adeno-associated viruses (AAVs)—the vector of choice in gene therapy—where protein ligands that resemble the Protein A used for mAb purification are employed at the product capture step.

The landscape of commercial affinity resins for AAV purification—now counting six adsorbents, including POROS™ CaptureSelect™ AAVX, AAV8 (CSAL8) and AAV9 (CSAL9), AVB Sepharose HP, Capto AVB, and AVIPure—uniformly relies on single-chain camelid antibody fragments derived from animal immunization or biological display libraries. While providing the high binding strength and selectivity needed to isolate AAVs from current feedstocks, which typically feature low product titer and a wide abundance of impurities, these ligands also require the use of harsh aqueous buffers (pH≤3) to release the bound capsids and limits the reusability of the resins, despite the high cost, due to their limited biochemical stability.

Leveraging the chemical and structural diversity of peptides has delivered a collection of binders for protein purification that combine high selectivity and capacity with mild elution conditions: recent examples include ligands that release the product upon exposure to mild pH (≥4), kosmotropic salts (MgCl₂), or light, thus safeguarding the bioactivity of labile targets. The adoption of peptide ligands, which can be mass manufactured rapidly and affordably, is also conducive to reducing the production costs of viral vectors. This is particularly à propos in the field of gene therapy, where manufacturing costs—to which the purification segment contributes a great deal—result in staggering price tags to patients, ranging between $2.5-3.5M.

### SUMMARY

Embodiments of the present disclosure include a composition for purifying an adeno-associated virus (AAV) from a biological fluid. In accordance with these embodiments, the composition comprises at least one peptide ligand that is at least ten amino acids in length and comprises an AAV-binding motif having at least 80% identity to one of the following: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21).

In some embodiments, the AAV is a recombinant AAV (rAAV).

In some embodiments, the at least one peptide ligand is cyclic.

In some embodiments, the AAV is selected from the group consisting of AAV1, AAV2, AAV3, AAV4, AAV5, AAV6, AAV7, AAV8, AAV9, and AAVrh10. In some embodiments, the AAV is selected from the group consisting of AAV1, AAV2, AAV5, AAV6, AAV8, and AAV9.

In some embodiments, the at least one peptide ligand comprises an AAV-binding motif having at least 80% identity with SEQ ID NO: 18, and further comprises an amino acid sequence having at least 80% identity with any one of SEQ ID NOs: 1, 2, 6, 7, and 14-16.

In some embodiments, the at least one peptide ligand comprises an AAV-binding motif having at least 80% identity with SEQ ID NO: 19, and further comprises an amino acid sequence having at least 80% identity with any one of SEQ ID NOs: 10-13, and 33-35.

In some embodiments, the at least one peptide ligand comprises an AAV-binding motif having at least 80% identity with SEQ ID NO: 20, and further comprises an amino acid sequence having at least 80% identity with any one of SEQ ID NOs: 3-5, 8, and 9.

In some embodiments, the at least one peptide ligand comprises an AAV-binding motif having at least 80% identity with SEQ ID NO: 21, and further comprises an amino acid sequence having at least 80% identity with any one of SEQ ID NO: 17.

In some embodiments, the at least one peptide ligand binds AAV1 by interacting with at least one of the following amino acids located on the solvent-accessible surface of the conserved regions of its capsid virion proteins (VP1, VP2, and VP3): THR504, ASN500, SER499, TRP503, ASN269, ASP270, SER268, ASN271, ALA267, GLY266, HIS272, SER262, SER385, ALA263, GLN386, GLY384, ASN383, ASN512, GLY513, and/or LYS508.

In some embodiments, the at least one peptide ligand binds AAV2 by interacting with at least one of the following amino acids located on the solvent-accessible surface of the conserved regions of its capsid virion proteins (VP1, VP2, and VP3): GLN385, THR503, GLU499, TRP502, LYS507, ASN268, ASP269, SER267, ALA266, GLY265, HIS271, SER384, SER264, GLN263, SER262, GLY383, ASN382, and/or ASN511.

In some embodiments, the at least one peptide ligand binds AAV5 by interacting with at least one of the following amino acids located on the solvent-accessible surface of the conserved regions of its capsid virion proteins (VP1, VP2, and VP3): SER319, GLU708, GLN532, PRO533, ASN535, TYR542, ALA534, ASN530, ASN546, ASP704, SER531, GLY545, ARG710, PHE698, MET547, THR711, GLU544, THR712, LEU548, ARG713, GLN697, LEU543, THR541, ALA540, THR538, and/or GLY478.

In some embodiments, the at least one peptide ligand binds AAV9 by interacting with at least one of the following amino acids located on the solvent-accessible surface of the conserved regions of its capsid virion proteins (VP1, VP2, and VP3): GLY266, ASN262, THR264, SER263, GLY267, GLU500, SER499, ASN498, PRO504, TRP503, SER269, ASN270, ASP271, SER268, SER386, ALA273, GLN387, ASP384, and/or GLY385.

In some embodiments, the at least one peptide ligand binds AAV2 by interacting with at least one of the following amino acids located on the solvent-accessible surface of the conserved regions of its capsid virion proteins (VP1, VP2, and VP3): THR716, ASN717, VAL719, VAL708, LYS706, ASN709, GLU548, LYS556, and/or ASP553.

In some embodiments, the at least one peptide ligand comprises more than one of the AAV-binding motif of SEQ ID NOs: 18-21. In some embodiments, the at least one peptide ligand comprises any combination of the AAV-binding motifs of SEQ ID NOs: 18-21.

In some embodiments, the at least one peptide ligand is no more than 25 amino acids in length.

In some embodiments, the at least one peptide ligand comprises an isoelectric point from about 3.5 to about 9.5.

In some embodiments, the at least one peptide ligand comprises a polarity value from about-1.2 to about 1.2.

In some embodiments, the at least one peptide ligand exhibits a disassociation constant (K_{D}) less than or equal to about 10⁻⁵ M at a pH that is higher than or equal to 7.0. In some embodiments, the at least one peptide ligand exhibits a disassociation constant (K_{D}) higher than or equal to about 10⁻⁴ M at a pH that is lower than or equal to 6.5.

In some embodiments, the at least one peptide ligand from (a) and/or (b) exhibits a dynamic binding capacity (DBC_{10%}) of at least 10¹³ vp/mL of resin.

In some embodiments, the at least one peptide ligand from (a) comprises: (i) an amino acid sequence having at least 80% sequence identity with SEQ ID NO: 1; (ii) an amino acid sequence having at least 80% sequence identity with SEQ ID NO: 2; (iii) an amino acid sequence having at least 80% sequence identity with SEQ ID NO: 3; (iv) an amino acid sequence having at least 80% sequence identity with SEQ ID NO: 4; (v) an amino acid sequence having at least 80% sequence identity with SEQ ID NO: 5; (vi) an amino acid sequence having at least 80% sequence identity with SEQ ID NO: 6; (vii) an amino acid sequence having at least 80% sequence identity with SEQ ID NO: 7; (viii) an amino acid sequence having at least 80% sequence identity with SEQ ID NO: 8; (ix) an amino acid sequence having at least 80% sequence identity with SEQ ID NO: 9; (x) an amino acid sequence having at least 80% sequence identity with SEQ ID NO: 10; (xi) an amino acid sequence having at least 80% sequence identity with SEQ ID NO: 11; (xii) an amino acid sequence having at least 80% sequence identity with SEQ ID NO: 12; (xiii) an amino acid sequence having at least 80% sequence identity with SEQ ID NO: 13; (xiv) an amino acid sequence having at least 80% sequence identity with SEQ ID NO: 14; (xv) an amino acid sequence having at least 80% sequence identity with SEQ ID NO: 15; (xvi) an amino acid sequence having at least 80% sequence identity with SEQ ID NO: 16; (xvii) an amino acid sequence having at least 80% sequence identity with SEQ ID NO: 17; (xviii) an amino acid sequence having at least 80% sequence identity with SEQ ID NO: 33; (xix) an amino acid sequence having at least 80% sequence identity with SEQ ID NO: 34; and/or (xx) an amino acid sequence having at least 80% sequence identity with SEQ ID NO: 35.

In some embodiments, the composition comprises: (i) at least two peptide ligands from SEQ ID NOs: 1-17 and 33-35; (ii) at least three peptide ligands from SEQ ID NOs: 1-17 and 33-35; (iii) at least four peptide ligands from SEQ ID NOs: 1-17 and 33-35; (iv) at least five peptide ligands from SEQ ID NOs: 1-17 and 33-35; (v) at least six peptide ligands from SEQ ID NOs: 1-17 and 33-35; (vi) at least seven peptide ligands from SEQ ID NOs: 1-17 and 33-35; (vii) at least eight peptide ligands from SEQ ID NOs: 1-17 and 33-35; (viii) at least nine peptide ligands from SEQ ID NOs: 1-17 and 33-35; (ix) at least ten peptide ligands from SEQ ID NOs: 1-17 and 33-35; (x) at least eleven peptide ligands from SEQ ID NOs: 1-17 and 33-35; (xi) at least twelve peptide ligands from SEQ ID NOs: 1-17 and 33-35; (xii) at least thirteen peptide ligands from SEQ ID NOs: 1-17 and 33-35; (xiii) at least fourteen peptide ligands from SEQ ID NOs: 1-17 and 33-35; (xiv) at least fifteen peptide ligands from SEQ ID NOs: 1-17 and 33-35; (xv) at least sixteen peptide ligands from SEQ ID NOs: 1-17 and 33-35; (xvi) at least seventeen peptide ligands from SEQ ID NOs: 1-17 and 33-35; (xvii) at least eighteen peptide ligands from SEQ ID NOs: 1-17 and 33-35; (xviii) at least nineteen peptide ligands from SEQ ID NOs: 1-17 and 33-35; or (xix) all twenty peptide ligands of SEQ ID NOs: 1-17 and 33-35.

In some embodiments, the at least one peptide ligand comprises a linker. In some embodiments, the at least one peptide ligand is bound to a solid support. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate and derivatives, polyolefin and derivatives, polyesters and derivatives, polyethers and derivatives, polystyrene, crosslinked polysaccharides, iron oxide, silica, titania, and/or zirconia.

In some embodiments, the biological fluid is a cell culture fluid. In some embodiments, the biological fluid comprises a supernatant and/or a cellular lysate. In some embodiments, the biological fluid is derived from a virus production cell line. In some embodiments, the virus production cell line is selected from the group consisting of CHO cells, HEK293 cells, MDCK-S, MDCK-A, Vero cells, LLC-MK2D, PER.C6, EB66, AGE1.CR cells, Spodoptera frugiperda (Sf9) cells, and HeLa cells, or any derivatives or variants thereof.

Embodiments of the present disclosure also include an adsorbent comprising any of the compositions described herein.

Embodiments of the present disclosure also include a method of purifying an adeno-associated virus (AAV) from a biological fluid. In accordance with these embodiments, the method comprising contacting the composition comprising any of the peptide ligands described herein (or any of the adsorbents described herein) with a biological fluid comprising the AAV, wherein the peptide ligand binds the AAV; and eluting the AAV from the peptide ligand.

In some embodiments, the elution is performed at pH from about 6.0 to about 7.5.

In some embodiments, a composition comprising at least one peptide ligand described herein, or an adsorbent comprising at least one peptide ligand described herein, is substantially resistant to NaOH, or a similar alkaline agent, at a concentration from about 0.1M to about 0.5M.

In some embodiments, a composition comprising at least one peptide ligand described herein, or an adsorbent comprising at least one peptide ligand described herein, is substantially reusable for up to about 10 cycles.

In some embodiments, the method further comprises a washing step before eluting the AAV from the at least one peptide ligand.

In some embodiments, the method results in at least a 50% yield for the AAV.

In some embodiments, the method produces at least an 80-fold reduction in host cell proteins.

Embodiments of the present disclosure also include an adeno-associated virus (AAV) purified using an of the methods described herein, wherein the AAV exhibits at least 25% transduction efficiency.

### BRIEF DESCRIPTION OF THE DRAWINGS

FIGS. 1A-1F: Representative complexes formed by A20-mimetic peptides CYIHFSGYTNYNPSLKSC (A1, red; SEQ ID NO: 1), CYGHFSGYGNYGPC (A4, green; SEQ ID NO: 4), CYIHFSGYTNYNPC (A6, blue; SEQ ID NO: 6), CVIDGSQSTDDDKIC (A10, yellow; SEQ ID NO: 10), CDGSQSTDDDKIC (A11, magenta), and LITHPRDYSPKLTPGLYEFG (A17, orange) with the capsids of (A) AAV1 (PDB IDs: 6JCQ, 6JCR, 7RK9, and 8FQ4), (B) AAV2 (5IPI, 6IH9, 6IHB, and 6U0V); (C) AAV5 (6JCS, 6JCT, 7KP3, and 7KPN); (D) AAV6 (3SHM, 3OAH, 4V86, and 5EGC); (E) AAV8 (2QA0, 3RAA, 6PWA, 6U2V, and 6V10); and (F) AAV9 (3UX1, 7MT0, 7WJW, and 7WJX) obtained via molecular docking and dynamics simulations at pH 7.4 and ionic strength of 150 mM. The AAVR and the A20 antibody are presented as light pink and light green cartoons, respectively; the VP1 is presented as light blue cartoon, while the remainder of the capsid is presented in light gray cartoon.

FIGS. 2A-2F: Values of loss (orange, calculated as the ratio of the AAV titer in the flow-through and wash fractions vs. load) and yield (green, calculated as the ratio of the AAV titer in the elution fraction vs. load) of (A) AAV1, (B) AAV2, (C) AAV5, (D) AAV6, (E) AAV8 and (F) AAV9 obtained via bind-and-elute studies in non-competitive mode using peptide-based resins (A1; SEQ ID NO: 1) CYIHFSGYTNYNPSLKSC-, (A3; SEQ ID NO: 3) CYVHFSGYSNYSPSC-, (A4; SEQ ID NO: 4) CYGHFSGYGNYGPC-, (A6; SEQ ID NO: 6) CYIHFSGYTNYNPC-, (A10; SEQ ID NO: 10) CVIDGSQSTDDDKIC- and (A12; SEQ ID NO: 12) CDSQSTDDDKIC-Toyopearl resins, and control adsorbents POROS™ CaptureSelect™ AAVX (AAVX) and AVB Sepharose HP (AVB) resins. The AAV titer in the flow-through, wash, and elution fractions was measured using serotype-specific ELISA kits.

FIGS. 3A-3B: Values of loss (orange, calculated as the ratio of the AAV titer in the flow-through and wash fractions vs. load) and yield (green, calculated as the ratio of the AAV titer in the elution fraction vs. load) of AAV2 and logarithmic reduction of HCPs (HCP LRV, red triangles) obtained via chromatographic purification of AAV2 from (A) a clarified HEK293 cell lysate (AAV2 titer: ~1.9·10¹² vp/mL; HCP titer: ~0.3 mg/mL) and (B) a clarified Sf9 cell lysate (AAV2 titer: ~1.56·10¹² vp/mL; HCP titer: ~1.1 mg/mL) using adsorbents (A1; SEQ ID NO: 1) CYIHFSGYTNYNPSLKS-, (A4; SEQ ID NO: 4) CYGHFSGYGNYGPC-, and (A10; SEQ ID NO: 10) CVIDGSQSTDDDKIC-Toyopearl resins together with control adsorbents POROS™ CaptureSelect™ AAVX (AAVX) and AVB Sepharose HP (AVB) resins. The AAV titer in the flow-through, wash, and elution fractions was measured using serotype-specific ELISA kits.

FIG. 4: Values of relative transduction efficiency of AAV2 purified from a clarified HEK293 cell lysate using peptide-based adsorbents (A1; SEQ ID NO: 1) CYIHFSGYTNYNPSLKS-, (A4; SEQ ID NO: 4) CYGHFSGYGNYGPC-, and (A10; SEQ ID NO: 10) CVIDGSQSTDDDKIC-Toyopearl resins together with control adsorbent POROS™ CaptureSelect™ AAVX resin (note: AAVX and AAVX* denote the eluate that was neutralized respectively immediately and 48 hrs after collection). The transduction efficiency (TU/vp) of eluted AAV2 was measured on human epithelial (HT1080; 10⁷ vp per cell) by performing a green fluorescence assay using a CytoFLEX Flow Cytometer. The values of relative transduction efficiency were calculated as the ratio of transduction efficiency of eluted AAV2 vs. AAV2 in the feedstock.

FIGS. 5A-5D: Contacts formed by (A) AAV1 in complex with AAVR (PDB ID: 6JCQ and 7T15), (B) AAV2 in complex with AAVR (6IHB and 6NZ0) and monoclonal antibody A20 (3J1S), (C) AAV5 in complex with AAVR (6JCS), and (D) AAV9 in complex with AAVR (7WJX); the full capsid in is gray cartoon, the target VP1 is in light blue cartoon, the AAVR is in light pink cartoon, and the A20 is in light green cartoon; the interacting residues on VP1 are in blue sticks, on AAVR are in magenta sticks, and on A20 are in green sticks.

FIGS. 6A-6C: Examples of complexes formed by peptides (A) CYIHFSGYTNYNPSLKSC (A1, blue cartoon; SEQ ID NO: 1), (B) CYGHFSGYGNYGPC (A4, green cartoon; SEQ ID NO: 4), and (C) CVIDGSQSTDDDKIC (A10, yellow cartoon; SEQ ID NO: 10) with the solvent accessible peptide segments displayed on the convex side of the VP1 protein of AAV1 (PDB ID: 6JCR), AAV2 (6IH9), AAV5 (7KP3), AAV6 (5EGC), AAV8 (2QA0), and AAV9 (7WJX). The segments of the VP that are not solvent accessible or whose homology among AAV serotypes is lower than 95% are in grey cartoon, the homologous segments of VP that are solvent accessible and displayed on the concave side of the capsid are in pink cartoon, and the homologous segments of VP that are solvent accessible and displayed on the convex side of the capsid are in red cartoon.

FIGS. 7A-7B: Chromatograms of AAV2 purification from a clarified HEK293 cell lysate (AAV2 titer: ~2.51·10¹² vp/mL; HCP titer: ~0.3 mg/mL) using peptide-based adsorbents (A1; SEQ ID NO: 1) CYIHFSGYTNYNPSLKSC-, (A3; SEQ ID NO: 3) CYVHFSGYSNYSPSC-, (A4; SEQ ID NO: 4) CYGHFSGYGNYGPC-, (A6; SEQ ID NO: 6) CYIHFSGYTNYNPC-, (A10; SEQ ID NO: 10) CVIDGSQSTDDDKIC- and (A12; SEQ ID NO: 12) CDSQSTDDDKIC-Toyopearl resins, and control adsorbents POROS™ CaptureSelect™ AAVX and AVB Sepharose HP resins. Binding was conducted in 20 mM NaCl in 10 mM Bis-Tris buffer at pH 7.0 (RT: 3 min); elution from the peptide-functionalized resins was conducted using 1 M MgCl₂ in 10 mM Bis-Tris buffer at pH 6.0 (RT: 1 min); elution from POROS™ CaptureSelect™ AAVX affinity resin (AAVX) and AVB Sepharose HP resin (AVB) was conducted using 0.2 M MgCl₂ in 200 mM citrate buffer at pH 2.2 and PBS at pH 2.0, respectively (RT: 2 min).

FIGS. 8A-8F: Size Exclusion Chromatography (SEC) analysis of (A) a clarified HEK293 cell lysate (AAV2 titer: ~2.51·10¹² vp/mL; HCP titer: ~0.3 mg/mL) and the elution fractions obtained from the purification of AAV2 from the clarified lysate using peptide-based adsorbents (B) (A1; SEQ ID NO: 1) CYIHFSGYTNYNPSLKSC-, (C) (A4; SEQ ID NO: 4) CYGHFSGYGNYGPC-, (D) (A10; SEQ ID NO: 10) CVIDGSQSTDDDKIC-Toyopearl resins, and control adsorbents (E) (AAVX) POROS™ CaptureSelect™ AAVX and (F) (AVB) AVB Sepharose HP resins.

FIGS. 9A-9F: Steric Exclusion Chromatography (SXC) analysis of (A) a clarified HEK293 cell lysate (AAV2 titer: ~2.51·10¹² vp/mL; HCP titer: ~0.3 mg/mL) and the elution fractions obtained from the purification of AAV2 from the clarified lysate using peptide-based adsorbents (B) (A1; SEQ ID NO: 1) CYIHFSGYTNYNPSLKSC-, (C) (A4; SEQ ID NO: 4) CYGHFSGYGNYGPC-, (D) (A10; SEQ ID NO: 10) CVIDGSQSTDDDKIC-Toyopearl resins, and control adsorbents (E) (AAVX) POROS™ CaptureSelect™ AAVX and (F) (AVB) AVB Sepharose HP resins.

FIGS. 10A-10G: Transmission Electron Microscopy (TEM) analysis of (A) a clarified HEK293 cell lysate (AAV2 titer: ~2.51·10¹² vp/mL; HCP titer: ~0.3 mg/mL) and the elution fractions obtained from the purification of AAV2 from the clarified lysate using peptide-based adsorbents (B) (A1; SEQ ID NO: 1) CYIHFSGYTNYNPSLKSC-, (C) (A3; SEQ ID NO: 3) CYVHFSGYSNYSPSC, (D) (A4; SEQ ID NO: 4) CYGHFSGYGNYGPC-, (E) (A10; SEQ ID NO: 10) CVIDGSQSTDDDKIC-, and (F) (A12; SEQ ID NO: 12) CDSQSTDDDKIC-Toyopearl resins. (G) Particle diameter distribution in the elution fraction obtained using peptide-based adsorbent (A10; SEQ ID NO: 10) CVIDGSQSTDDDKIC-Toyopearl resin.

FIG. 11: Breakthrough curve obtained by loading a clarified HEK293 cell lysate (AAV2 titer: ~2.51·10¹² vp/mL; HCP titer: ~0.3 mg/mL) on CVIDGSQSTDDDKIC-Toyopearl (SEQ ID NO: 10) resins at the residence time of 3 min; the curve was obtained by fitting the values of AAV2 titer in the effluent measured via AAV2 ELISA kits. Figure onset: values of dynamic AAV2 binding capacity (DBC_{10%}) of peptide-functionalized resins loaded (RT: 3 min) with a clarified HEK293 cell lysate containing AAV2 at the titer of 2.51·10¹² vp/mL.—: not available.

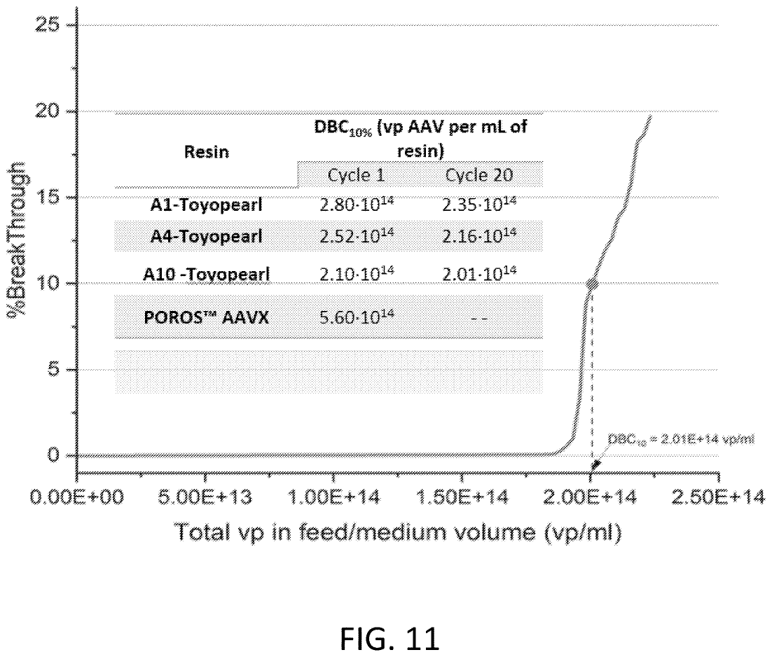

FIGS. 12A-12D: Values of loss (blue, calculated as the ratio of the AAV titer in the flow-through and wash fractions vs. load) and yield (green, calculated as the ratio of the AAV titer in the elution fraction vs. load) of AAV and logarithmic reduction of HCPs (HCP LRV, red triangles) obtained via chromatographic purification of AAV from clarified HEK293 cell lysates at pH 7.4 containing (A) AAV3 (capsid titer: ~5.0·10¹¹ vp/mL; HCP titer: ~0.2 mg/mL), (B) AAV6 (capsid titer: ~1.0·10¹¹ vp/mL; HCP titer: ~0.2 mg/mL), (C) AAV9 (capsid titer: ~5.0·10¹¹ vp/mL; HCP titer: ~0.2 mg/mL), and (D) AAVrh.10 (capsid titer: ~1.0·10¹¹ vp/mL; HCP titer: ~0.2 mg/mL) using CYIHFSGYTNYNPSLKSC (A1; SEQ ID NO: 1), CYVHFSGYSNYSPSC (A3; SEQ ID NO: 3), CYGHFSGYGNYGPC (A4; SEQ ID NO: 4), CYIHFSGY-TNYNPC (A6; SEQ ID NO: 6), CVIDGSQSTDDDKIC (A10; SEQ ID NO: 10), CDGSQSTDDDKIC (A11; SEQ ID NO: 11), CDSQSTDDDKIC (A12; SEQ ID NO: 12), and CSGSTDDDKIC (A13; SEQ ID NO: 13) Toyopearl resins together with control Poros™ CaptureSelect™ AAVX (AAVX) and AVB Sepharose HP (AVB) resins. The AAV and HEK293 HCP titers in the flow-through, wash, and elution fractions were measured using serotype-specific and HEK293 HCP ELISA kits, respectively.

FIGS. 13A-13D: Values of loss (blue, calculated as the ratio of the AAV titer in the flow-through and wash fractions vs. load) and yield (green, calculated as the ratio of the AAV titer in the elution fraction vs. load) of AAV and logarithmic reduction of HCPs (HCP LRV, red triangles) obtained via chromatographic purification of AAV from clarified HEK293 cell lysate containing (A) AAV1 (capsid titer: ~3.0·10¹¹ vp/mL; HCP titer: ~0.11 mg/mL), (B) AAV5 (capsid titer: ~1.1·10¹² vp/mL; HCP titer: ~0.16 mg/mL), (C) AAV7 (capsid titer: ~7.4·10¹¹ vp/mL; HCP titer: ~0.18 mg/mL), and (D) AAV8 (capsid titer: ~1.6·10¹¹ vp/mL; HCP titer: ~0.17 mg/mL) using CVIDGSQSTDDDKIC (A10; SEQ ID NO: 10), CDGSQSTDDDKIC (A11; SEQ ID NO: 11), CDSQSTDDDKIC (A12; SEQ ID NO: 12), and CSGSTDDDKIC (A13; SEQ ID NO: 13) Toyopearl resins together with control Poros™ CaptureSelect™ AAVX (AAVX) and AVB Sepharose HP (AVB) resins. The AAV and HEK293 HCP titers in the flow-through, wash, and elution fractions were measured using serotype-specific and HEK293 HCP ELISA kits, respectively.

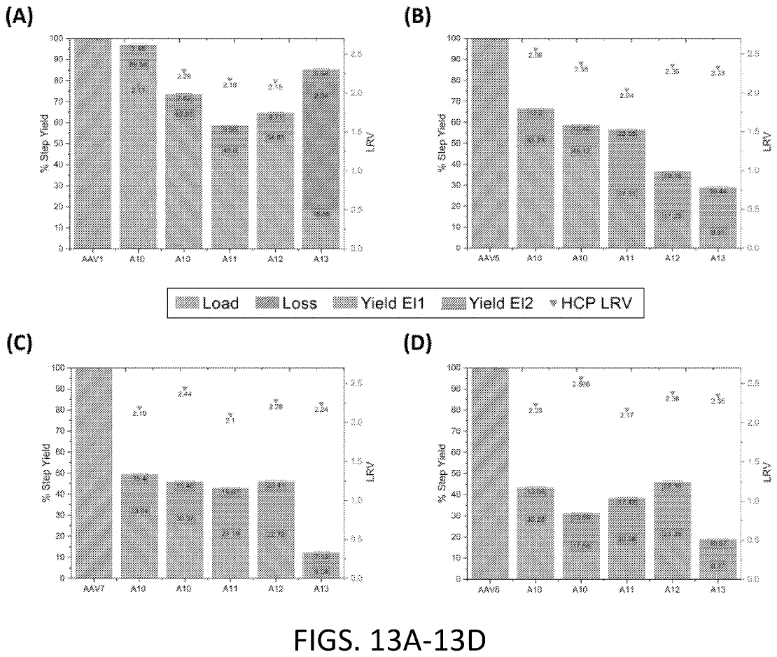

FIGS. 14A-14D: Values of total viral genomes (orange, measured via qPCR), total transducing units (green, measured via transduction assay), and step yield (black star, calculated as the ratio of the AAV titer in the elution fraction vs. load) obtained via chromatographic purification of AAV from clarified HEK293 cell lysates at pH 7.4 containing (A) AAV3 (capsid titer: ~5.0·10¹¹ vp/mL; HCP titer: ~0.2 mg/mL), (B) AAV6 (capsid titer: ~1.0·10¹¹ vp/mL; HCP titer: ~0.2 mg/mL), (C) AAV9 (capsid titer: ~5.0·10¹¹ vp/mL; HCP titer: ~0.2 mg/mL), and (D) AAVrh.10 (capsid titer: ~1.0·10¹¹ vp/mL; HCP titer: ~0.2 mg/mL) using CVIDGSQSTDDDKIC (A10; SEQ ID NO: 10) Toyopearl resin.

FIGS. 15A-15C: Values of total viral genomes (orange, measured via qPCR), total transducing units (green, measured via transduction assay), and step yield (black star, calculated as the ratio of the AAV titer in the elution fraction vs. load) obtained via chromatographic purification of AAV from clarified HEK293 cell lysates at pH 7.4 containing (A) AAV1 (capsid titer: ~3.0·10¹¹ vp/mL; HCP titer: ~0.11 mg/mL), (B) AAV7 (capsid titer: ~7.4·10¹¹ vp/mL; HCP titer: ~0.18 mg/mL), and (C) AAV8 (capsid titer: ~1.6·10¹¹ vp/mL; HCP titer: ~0.17 mg/mL) using CVIDGSQSTDDDKIC (A10; SEQ ID NO: 10) Toyopearl resin.

FIGS. 16A-16D: Representative complexes formed by CVIDGQASTDDDKIC (A10, red cartoon; SEQ ID NO: 10), CVIDGSASTDDDRIC (A10′, green cartoon; SEQ ID NO: 33), CVIDGSSSTDDDRIC (A10″, blue cartoon; SEQ ID NO: 34), and CVIDGSASTDDDHIC (A10″, magenta cartoon; SEQ ID NO: 35) with the capsids of (A) AAV2 (PDB ID: 5IPI, 6IH9, 6IHB, and 6U0V); (B) AAV5 (6JCS, 6JCT, 7KP3, and 7KPN); (C) AAV8 (2QA0, 3RAA, 6PWA, 6U2V, and 6V10); and (D) AAV9 (3UX1, 7MT0, 7WJW, and 7WJX). The AAVR are presented as light pink cartoon. The interdigitated VP proteins are presented as light blue cartoon, while the remainder of the capsid is presented in light gray cartoon.

FIG. 17: Consecutive purification of AAV9 from clarified HEK293 cell lysates using A10′-Poros™ resins with intermediate CIP using 0.1 M NaOH (15 CVs at the RT of 1 min followed by 15 min of static contact time).

FIGS. 18A-18H: Chromatograms of AAV purification from (A) a clarified AAV1-HEK293 cell lysate (AAV1 titer: ~3.0·10¹¹ vp/mL; HCP titer: ~0.11 mg/mL); (B) a clarified HEK293 cell lysate (AAV3 titer ~5.0·10¹¹ vp/mL; HCP titer ~0.2 mg/mL); (C) a clarified AAV5-HEK293 cell lysate (AAV5 titer: ~1.1·10¹² vp/mL; HCP titer: ~0.16 mg/mL); (D) a clarified HEK293 cell lysate (AAV6 titer: ~1.0·10¹¹ vp/mL; HCP titer: ~0.2 mg/mL); (E) a clarified AAV7-HEK293 cell lysate (AAV7 titer: ~7.4·10¹¹ vp/mL; HCP titer: ~0.18 mg/mL); (F) a clarified AAV8-HEK293 cell lysate (AAV8 titer: ~1.6·10¹¹ vp/mL; HCP titer: ~0.17 mg/mL); (G) a clarified HEK293 cell lysate (AAV9 titer: ~5.0·10¹¹ vp/mL; HCP titer: ~0.2 mg/mL); and (H) a clarified HEK293 cell lysate (AAVrh10 titer: ~1.0·10¹¹ vp/mL; HCP titer: ~0.2 mg/mL) using peptide-based adsorbents (A1; SEQ ID NO: 1) CYIHFSGYTNYNPSLKSC-, (A3; SEQ ID NO: 3) CYVHFSGYSNYSPSC-, (A4; SEQ ID NO: 4) CYGHFSGYGNYGPC-, (A6; SEQ ID NO: 6) CYIHFSGYTNYNPC-, (A10; SEQ ID NO: 10) CVIDGSQSTDDDKIC-, (A11; SEQ ID NO: 11) CDGSQSTDDDKIC-, (A12; SEQ ID NO: 12) CDSQSTDDDKIC-, (A13; SEQ ID NO: 13) CSGSTDDDKIC-TP750F resins, and control adsorbents POROS™ CaptureSelect™ AAVX and AVB Sepharose HP resins. Serotypes AAV3, AAV6, AAV9, and AAVrh10 were bound in 20 mM NaCl in 10 mM Bis-Tris buffer at pH 7.0 (RT: 3 min) and eluted using 0.4 M MgCl₂ in 10 mM Bis-Tris buffer at pH 6.0 (RT: 1 min). Serotypes AAV1, AAV5, AAV7, and AAV8 were bound in 2 mM MgCl₂ in 50 mM Sodium Acetate buffer at pH 5.0 (RT: 3 min) and eluted using 20 mM NaCl in 10 mM Bis-Tris buffer at pH 7.0 (RT: 1 min); and 0.4 M MgCl₂ in 10 mM Bis-Tris buffer at pH 6.0 (RT: 1 min). Elution from POROS™ CaptureSelect™ AAVX affinity resin and AVB Sepharose HP resin was conducted using 0.2 M MgCl2 in 200 mM citrate buffer at pH 2.2 and PBS at pH 2.0, respectively (RT: 2 min).

FIGS. 19A-19H: Size Exclusion Chromatography (SEC) analysis of clarified HEK293 cell lysates (red) and the corresponding elution fraction (blue and purple) obtained by purifying (A) AAV1, (B) AAV2, (C) AAV3, (D) AAV5, (E) AAV7, (F) AAV8, (G) AAV9, and (H) AAVrh.10 using peptide-based adsorbent (A10; SEQ ID NO: 10) CVIDGSQSTDDDKIC Toyopearl resin.

FIGS. 20A-20H: Steric Exclusion Chromatography (SXC) analysis of clarified HEK293 cell lysates (red) and the corresponding elution fraction (blue and purple) obtained by purifying (A) AAV1, (B) AAV2, (C) AAV3, (D) AAV5, (E) AAV7, (F) AAV8, (G) AAV9, and (H) AAVrh.10 using peptide-based adsorbent (A10; SEQ ID NO: 10) CVIDGSQSTDDDKIC Toyopearl resin.

FIGS. 21A-21D: Values of loss (blue, calculated as the ratio of the AAV titer in the flow-through and wash fractions vs. load) and yield (green, calculated as the ratio of the AAV titer in the elution fraction vs. load) of AAV and logarithmic reduction of HCPs (HCP LRV, red triangles) obtained via chromatographic purification of AAV from clarified HEK293 cell lysates at pH 7.4 containing (A) AAV1 (capsid titer: ~3.0·10¹¹ vp/mL; HCP titer: ~0.11 mg/mL), (B) AAV5 (capsid titer: ~1.1·10¹² vp/mL; HCP titer: ~0.16 mg/mL), (C) AAV7 (capsid titer: ~7.4·10¹¹ vp/mL; HCP titer: ~0.18 mg/mL), and (D) AAV8 (capsid titer: ~1.6·10¹¹ vp/mL; HCP titer: ~0.17 mg/mL) using CYIHFSGYTNYNPSLKSC (A1; SEQ ID NO: 1), CYVHFSGYSNYSPSC (A3; SEQ ID NO: 3), CYGHFSGYGNYGPC (A4; SEQ ID NO: 4), CYIHFSGY-TNYNPC (A6; SEQ ID NO: 6), CVIDGSQSTDDDKIC (A10; SEQ ID NO: 10), CDGSQSTDDDKIC (A11; SEQ ID NO: 11), CDSQSTDDDKIC (A12; SEQ ID NO: 12), and CSGSTDDDKIC (A13; SEQ ID NO: 13) Toyopearl resins together with control Poros™ CaptureSelect™ AAVX (AAVX) and AVB Sepharose HP (AVB) resins. The AAV and HEK293 HCP titers in the flow-through, wash, and elution fractions were measured using serotype-specific and HEK293 HCP ELISA kits, respectively.

FIG. 22: Size Exclusion Chromatography (SEC) analysis of clarified HEK293 cell lysates (red) and the corresponding elution fraction (blue and purple) obtained by conducting consecutive purifications of AAV9 from clarified HEK293 cell lysates using A10′-Poros™ resins with intermediate CIP using 0.1 M NaOH (15 CVs at the RT of 1 min followed by 15 min of static contact time).

### DETAILED DESCRIPTION

In the dynamic landscape of gene therapy, Adeno-Associated Viruses (AAV) have emerged as a prominent class of delivery vectors, demonstrating remarkable potential for treating various genetic disorders. Recent years have witnessed a significant growth in the field, marked by the approval of several AAV-based products (i.e., Hemgenix, Zolgensma, Luxturna, Roctavian, and Elevidys) that are revolutionizing the landscape of therapeutic interventions. These products highlight the clinical success and growing acceptance of viral vectors as safe and effective delivery vehicles of therapeutic and vaccine genes.

The future of AAVs—and gene and cell therapies in general—relies on the efficiency and precision of both upstream and downstream bioprocessing. The upstream segment, comprising vector production, plays a pivotal role in generating high-quality AAV vectors with desired critical quality attributes. Downstream processing—articulated in an affinity purification step and a full-capsid enrichment step—ensures the isolation of pure and potent AAV vectors from complex cell lysate feedstocks. The synergy between these two segments is crucial to achieve scalable, reproducible, and cost-effective vector production. The affinity purification of AAV is a critical step in the production process and one that almost ubiquitously relies on affinity chromatography. This technique leverages the specific interactions between ligands and viral capsid proteins, allowing for efficient and selective purification.

Recent commercial releases have focused on peptide-based affinity ligands. First came the AVIPure® AAV9 affinity resin, launched by Repligen, which utilizes a cyclic peptide featuring high AAV9 selectivity and resistance to alkaline sanitization. Later, LigaTrap Technologies introduced LigaGuard™ and AA Vidity™ resins for the purification of AAVs in flow-through and bind-and-elute mode respectively. In this context, embodiments of the present disclosure were designed as cyclic peptide mimetics of the AAV receptor (AAVR) and are the first truly serotype-agnostic affinity ligands for AAV purification. The rationale of abstracting peptide sequences from AAVR, which is required for AAV serotypes 1, 2, 3, 5, 6, 8, and 9 to achieve cell transduction, and further modifying them to expand their biorecognition range aimed at identifying ligands that selectively capture all AAVs in complex fluids. This technology was initially demonstrated by purifying AAV serotypes 2 and 6 from HEK293 and Sf9, affording high product capture (~2·10¹⁴ vp per mL of resin), yield and transduction activity (up to 70% and 60%, respectively), and purity (up to 700-fold reduction of HCPs). Among the tested candidates, CVIDGSQSTDDDKIC (A10; SEQ ID NO: 10) was chosen for additional characterization.

A unique property of AAVR-mimetic peptides is their nuanced binding of the various AAV serotypes, which stems from the unique amino acid sequence and structural features of the respective virion proteins. It was observed that A10 binding occurs at neutral pH for serotypes 2, 3, 6, 9, and rh10, whereas serotypes 1, 5, 7, and 8 required a slightly acidic environment (pH 5). This mirrors the AAVR: AAV interactions, which show a similar pH dependence. Nonetheless, the bound AAVs are consistently released in near-physiological conditions upon contact with magnesium chloride, ensuring the recovery of AAVs with high transduction activity. Among other important insights, the present disclosure provides an in-depth evaluation of ligand A10 and ancillary peptides conjugated to different chromatographic resins by demonstrating the purification of all major AAV serotypes from HEK293 cell lysates and comparing its purification outcomes (i.e., AAV binding capacity, capsid and genome yield, and removal of host cell proteins and nucleic acids) in comparison with commercial affinity resins. Complementing the purification performance is the chemical stability of the peptide ligands, which enable conducting multiple purification cycles with intermediate caustic cleaning without loss of product yield or purity, enabling the long-term reusability that is much needed to decrease the manufacturing cost of gene therapies.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “chromatography,” as used herein, refers to a technique which separates the product of interest (e.g., AAVs) from contaminants and/or protein aggregates in a biopharmaceutical preparation.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the terms “purified” and “purification” refer to the removal of contaminants or impurities from a sample, for example, the removal of process-related impurities (e.g., soluble molecules that are not the product or derived from the product) and/or product-related imputes (e.g., soluble molecules that are derived from the product but they are not identical to the product in terms of amino acid sequence, tertiary/quaternary structure, post-translational modifications, and functional activity). In some aspects, AVVs are purified by removing various process-related impurities such as proteins, DNA and RNA, soluble components generated/released by cells, and various other soluble components present in the cell culture media; and product-related impurities such as fragmented, incomplete, or inactive viral capsids, or viral capsids that contain no or incorrect gene payload. In another example, AAVs can be expressed in host cells and purified by the removal of host cell proteins; the amount of AAVs is thereby increased in the sample.

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an antibody or any antigen binding fragment/derivative thereof (e.g., monoclonal or polyclonal antibody). In other embodiments, the target or target biologic is a viral vector (e.g., AAV).

As used herein, the term “sample” refers to any composition or mixture that contains a target biologic (e.g., AAV). Samples may be derived from biological or other sources. Biological sources include prokaryotic sources and eukaryotic sources, the latter including fungal, vegetal, and animal cells, tissues, and organs. The sample may also include diluents, buffers, additives, and contaminating species, debris and the like that are found mixed with the target biologic. The sample may be “partially purified” (e.g., having been subjected to one or more purification steps, such as filtration steps) or may be obtained directly from a host cell or organism producing the target molecule (e.g., the sample may comprise harvested cell culture fluid).

As used herein, the term “target” or “target biologic” generally refers to a target protein, peptide, polypeptide, nucleic acid, ribonucleoprotein complex, nucleic acid construct, supramolecular construct, virus, viral construct, virus-like particle, cell, organelle, small molecule, and any combinations thereof, which may be present in a sample (e.g., biological fluid) comprising one or more process-related impurities and/or product-related substances. In some embodiments, the target or target biologic is an AAV.

As used herein, the term “host cell protein” or “HCP” refers to any intracellular or secreted protein produced by the organism engineered to produce the target biologic and unrelated to the target biologic. HCPs are generally undesirable in the final drug substance.

As used herein, a “mixture” comprises a target biologic of interest (for which purification is desired) and one or more contaminant or impurity. In some embodiments, the mixture is produced from a host cell or organism that expresses the AAV (either naturally or recombinantly). Such mixtures include, for example, cell cultures, cell lysates, and clarified bulk (e.g., clarified cell culture supernatant).

### 2. Compositions and Methods for Removing Process and Product-Related Impurities

a. Compositions

Given the importance and growth of AAVs, amply documented by the surge in clinical trials and the uptick in regulatory approvals, experiments were conducted to develop AAV-targeting peptides. Relevant to this endeavor has been the wealth of data on AAV-targeting biomolecules—particularly the crystal structures of the complexes formed by AAVs of different serotypes with the AAV receptor (AAVR) and the anti-AAV antibody A20—that has become available in the last decade. Accordingly, using rational design principles, cyclic peptide mimetics of AAVR and A20 were developed by abstracting sequences that target regions of the capsids that are highly conserved across serotypes of different clades. At the same time, to overcome the limitations of protein ligands, the peptide mimetics were designed to form complexes with different AAV serotypes that (i) feature high-affinity at physiological conditions (|ΔG_{b}|≥6.5 kcal/mol at pH 7.4) and (ii) undergo a ≥100-fold loss of binding strength as the pH is lowered to 6.5. Among the selected sequences, A20-mimetic YIHFSGYTNYNPSLKS (SEQ ID NO: 1) and AAVR-mimetic VIDGSQSTDDDKI (SEQ ID NO: 10) demonstrated excellent capture of serotypes belonging to distinct clones/clades—AAV1, AAV2, AAV5, AAV6, AAV8, and AAV9—corroborating the in silico models documenting their ability to target regions of the virion proteins that are conserved across all serotypes. VIDGSQSTDDDKI-Toyopearl resin (SEQ ID NO: 10) features values of binding capacity (~10¹⁴ vp per mL) and product yields (~60-80%) on par with commercial adsorbents, and purified AAV2 from a HEK293 cell lysate affording high recovery (70-80%), a 700-fold reduction of host cell proteins (HCPs), and high transduction activity (up to 65%) of the purified viruses.

In accordance with these embodiments, the present disclosure provides compositions and methods for purifying an adeno-associated virus (AAV) from a biological fluid. In some embodiments, the composition comprises at least one peptide ligand that is at least ten amino acids in length and comprises an AAV-binding motif having at least 80% identity to one of the following: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21). In some embodiments, the composition comprises at least one peptide ligand that is at least ten amino acids in length and comprises an AAV-binding motif having at least 85% identity to one of the following: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21). In some embodiments, the composition comprises at least one peptide ligand that is at least ten amino acids in length and comprises an AAV-binding motif having at least 90% identity to one of the following: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21). In some embodiments, the composition comprises at least one peptide ligand that is at least ten amino acids in length and comprises an AAV-binding motif having at least 95% identity to one of the following: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21). In some embodiments, the composition comprises at least one peptide ligand that is at least ten amino acids in length and comprises at least one of the following an AAV-binding motifs: (a) YIHFSGYT (SEQ ID NO: 18); (b) STDDD (SEQ ID NO: 19); (c) CYHFS (SEQ ID NO: 20); and/or (d) LITHPRDYS (SEQ ID NO: 21).

In some embodiments, the at least one peptide ligand of the present disclosure is cyclic. As would be recognized by one of ordinary skill in the art based on the present disclosure, cyclic peptides or polypeptides are molecules that are primarily composed of a peptide segment modified via head-to-tail, head-to-side-chain, side-chain-to-tail, or side-chain-to-side-chain cyclization. The peptide ligands of the present disclosure can be cyclized by any method available to one of skill in the art. For example, the N-terminal and C-terminal ends can be condensed to form a peptide bond by known procedures. Functional groups present on the side chains of amino acids in the peptides can also be joined to cyclize the peptides of the present disclosure. For example, functional groups that can form covalent bonds include —COOH and —OH; —COOH and —NH₂; and —COOH and —SH. Pairs of amino acids that can be used to cyclize a peptide include, Asp and Lys; Glu and Lys; Asp and Arg; Glu and Arg; Asp and Ser; Glu and Ser; Asp and Thr; Glu and Thr; Asp and Cys; and Glu and Cys. Other examples of amino acid residues that are capable of forming covalent linkages with one another include cysteine-like amino acids such Cys, hCys, β-methyl-Cys and Penicillamine (Pen), a non-proteinogenic an alpha-amino acid having the structure of valine substituted at the beta position with a sulfanyl group, which can form disulfide bridges with one another. Preferred cysteine-like amino acid residues include Cys and Pen. Other pairs of amino acids that can be used for cyclization of the peptide will be apparent to those skilled in the art. In accordance with this, the sequences of the peptide ligands of the present disclosure can be represented as “C-[sequence]-C” or a version thereof (e.g., “cycloCC[SEQUENCE]”), in which the primary sequence of the peptide ligand is flanked by Cys residues.

The groups used to cyclize a peptide need not be amino acids. Examples of functional groups capable of forming a covalent linkage with the amino terminus of a peptide include carboxylic acids and esters. Examples of functional groups capable of forming a covalent linkage with the carboxyl terminus of a peptide include —OH, —SH, —NH₂ and —NHR where R is (C₁-C₆) alkyl, (C₁-C₆)alkenyl and (C₁-C₆)alkynyl. In some embodiments, the N-terminal amino acid is covalently linked to the C-terminal amino acid in the cyclic peptide. In some embodiments, the covalent linkage is head to tail between the free C-terminal carboxyl and the free N-terminal amine. In some embodiments, the covalent linkage involves a side chain of the N-terminal amino acid, a side chain of the C-terminal amino acid, or both. In some embodiments, the N-terminal amino acid is covalently linked to the C-terminal amino acid with a thioether bond.

Preferably, the reaction conditions used to cyclize the peptides are sufficiently mild so as not to degrade or otherwise damage the peptide. Suitable groups for protecting the various functionalities as necessary are well known in the art (see, e.g., Greene & Wuts, 1991, 2nd ed., John Wiley & Sons, NY), as are various reaction schemes for preparing such protected molecules. In a preferred conventional procedure, the cyclic peptides of the present invention may be synthesized by solid-phase synthesis and purified according to methods known in the art. Any of a number of well-known procedures utilizing a variety of resins and reagents may be used to prepare the cyclic peptides of the present invention. In some embodiments, the peptides are modified to stabilize them, to facilitate their uptake and/or absorption, or to improve any other characteristic or property of the peptides that is known to one of skill in art. For example, charges on the peptides can be neutralized and the peptides can be linked to other chemical moieties. The peptides may also be modified by the addition of: radioactive atoms; detectable labels (e.g., radioactive labels, dyes, fluorescent moieties, chemiluminescent moieties, quantum dots); affinity tags (e.g., His tag, biotin); PEG moieties; carbohydrates (e.g., glycosylation, hesylation); and organic molecules (e.g., alkylation, acetylation, acylation).

The present disclosure also provides for nonpeptide compounds that mimic peptide sequences, synthesis of which are known in the art. Peptide mimetics that are structurally related to therapeutically useful peptides may be used to produce an equivalent or enhanced therapeutic or prophylactic effect. Generally, peptidomimetics are structurally similar to the peptide of interest, but have one or more peptide linkages optionally replaced by linkages such as —CH₂NH—, —CH₂S—, —CH₂CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such polypeptide mimetics may have significant advantages over naturally occurring polypeptides including more economical production, greater chemical stability, enhanced pharmacological properties (e.g., half-life, absorption, potency, efficiency), reduced antigenicity, and the like.

In some embodiments, the AAV is a recombinant AAV (rAAV). In some embodiments, the AAV is selected from the group consisting of AAV1, AAV2, AAV3, AAV4, AAV5, AAV6, AAV7, AAV8, AAV9, and AAVrh10. In some embodiments, the AAV is selected from the group consisting of AAV1, AAV2, AAV5, AAV6, AAV8, and AAV9.

In some embodiments, the at least one peptide ligand of the present disclosure comprises an AAV-binding motif having at least 80% identity with SEQ ID NO: 18, and further comprises an amino acid sequence having at least 80% identity with any one of SEQ ID NOs: 1, 2, 6, 7, and 14-16. In some embodiments, the at least one peptide ligand of the present disclosure comprises an AAV-binding motif having at least 85% identity with SEQ ID NO: 18, and further comprises an amino acid sequence having at least 85% identity with any one of SEQ ID NOs: 1, 2, 6, 7, and 14-16. In some embodiments, the at least one peptide ligand of the present disclosure comprises an AAV-binding motif having at least 90% identity with SEQ ID NO: 18, and further comprises an amino acid sequence having at least 90% identity with any one of SEQ ID NOs: 1, 2, 6, 7, and 14-16. In some embodiments, the at least one peptide ligand of the present disclosure comprises an AAV-binding motif having at least 95% identity with SEQ ID NO: 18, and further comprises an amino acid sequence having at least 95% identity with any one of SEQ ID NOs: 1, 2, 6, 7, and 14-16.

In some embodiments, the at least one peptide ligand of the present disclosure comprises an AAV-binding motif having at least 80% identity with SEQ ID NO: 19, and further comprises an amino acid sequence having at least 80% identity with any one of SEQ ID NOs: 10-13, and 33-35. In some embodiments, the at least one peptide ligand of the present disclosure comprises an AAV-binding motif having at least 85% identity with SEQ ID NO: 19, and further comprises an amino acid sequence having at least 85% identity with any one of SEQ ID NOs: 10-13, and 33-35. In some embodiments, the at least one peptide ligand of the present disclosure comprises an AAV-binding motif having at least 90% identity with SEQ ID NO: 19, and further comprises an amino acid sequence having at least 90% identity with any one of SEQ ID NOs: 10-13, and 33-35. In some embodiments, the at least one peptide ligand of the present disclosure comprises an AAV-binding motif having at least 95% identity with SEQ ID NO: 19, and further comprises an amino acid sequence having at least 95% identity with any one of SEQ ID NOs: 10-13, and 33-35.

In some embodiments, the at least one peptide ligand of the present disclosure comprises an AAV-binding motif having at least 80% identity with SEQ ID NO: 20, and further comprises an amino acid sequence having at least 80% identity with any one of SEQ ID NOs: 3-5, 8, and 9. In some embodiments, the at least one peptide ligand of the present disclosure comprises an AAV-binding motif having at least 85% identity with SEQ ID NO: 20, and further comprises an amino acid sequence having at least 85% identity with any one of SEQ ID NOs: 3-5, 8, and 9. In some embodiments, the at least one peptide ligand of the present disclosure comprises an AAV-binding motif having at least 90% identity with SEQ ID NO: 20, and further comprises an amino acid sequence having at least 90% identity with any one of SEQ ID NOs: 3-5, 8, and 9. In some embodiments, the at least one peptide ligand of the present disclosure comprises an AAV-binding motif having at least 95% identity with SEQ ID NO: 20, and further comprises an amino acid sequence having at least 95% identity with any one of SEQ ID NOs: 3-5, 8, and 9.

In some embodiments, the at least one peptide ligand of the present disclosure comprises an AAV-binding motif having at least 80% identity with SEQ ID NO: 21, and further comprises an amino acid sequence having at least 80% identity with any one of SEQ ID NO: 17. In some embodiments, the at least one peptide ligand of the present disclosure comprises an AAV-binding motif having at least 85% identity with SEQ ID NO: 21, and further comprises an amino acid sequence having at least 85% identity with any one of SEQ ID NO: 17. In some embodiments, the at least one peptide ligand of the present disclosure comprises an AAV-binding motif having at least 90% identity with SEQ ID NO: 21, and further comprises an amino acid sequence having at least 90% identity with any one of SEQ ID NO: 17. In some embodiments, the at least one peptide ligand of the present disclosure comprises an AAV-binding motif having at least 95% identity with SEQ ID NO: 21, and further comprises an amino acid sequence having at least 95% identity with any one of SEQ ID NO: 17.

In some embodiments, the at least one peptide ligand binds AAV1 by interacting with at least one of the following amino acids located on the solvent-accessible surface of the conserved regions of its capsid virion proteins (VP1, VP2, and VP3): THR504, ASN500, SER499, TRP503, ASN269, ASP270, SER268, ASN271, ALA267, GLY266, HIS272, SER262, SER385, ALA263, GLN386, GLY384, ASN383, ASN512, GLY513, and/or LYS508 (see, e.g., Table 8). In some embodiments, the at least one peptide ligand binds AAV2 by interacting with at least one of the following amino acids: GLN385, THR503, GLU499, TRP502, LYS507, ASN268, ASP269, SER267, ALA266, GLY265, HIS271, SER384, SER264, GLN263, SER262, GLY383, ASN382, and/or ASN511 (see, e.g., Table 8). In some embodiments, the at least one peptide ligand binds AAV5 by interacting with at least one of the following amino acids located on the solvent-accessible surface of the conserved regions of its capsid virion proteins (VP1, VP2, and VP3): SER319, GLU708, GLN532, PRO533, ASN535, TYR542, ALA534, ASN530, ASN546, ASP704, SER531, GLY545, ARG710, PHE698, MET547, THR711, GLU544, THR712, LEU548, ARG713, GLN697, LEU543, THR541, ALA540, THR538, and/or GLY478 (see, e.g., Table 8). In some embodiments, the at least one peptide ligand binds AAV9 by interacting with at least one of the following amino acids located on the solvent-accessible surface of the conserved regions of its capsid virion proteins (VP1, VP2, and VP3): GLY266, ASN262, THR264, SER263, GLY267, GLU500, SER499, ASN498, PRO504, TRP503, SER269, ASN270, ASP271, SER268, SER386, ALA273, GLN387, ASP384, and/or GLY385 (see, e.g., Table 8). In some embodiments, the at least one peptide ligand binds AAV2 by interacting with at least one of the following amino acids located on the solvent-accessible surface of the conserved regions of its capsid virion proteins (VP1, VP2, and VP3): THR716, ASN717, VAL719, VAL708, LYS706, ASN709, GLU548, LYS556, and/or ASP553 (see, e.g., Table 8).

In some embodiments, the at least one peptide ligand comprises more than one of the AAV-binding motif of SEQ ID NOs: 18-21. In some embodiments, the at least one peptide ligand comprises any combination of the AAV-binding motifs of SEQ ID NOs: 18-21.

In some embodiments, the at least one peptide ligand is no more than 20 amino acids in length. In some embodiments, the at least one peptide ligand is no more than 25 amino acids in length. In some embodiments, the at least one peptide ligand is no more than 30 amino acids in length. In some embodiments, the at least one peptide ligand is no more than 35 amino acids in length. In some embodiments, the at least one peptide ligand is no more than 40 amino acids in length. In some embodiments, the at least one peptide ligand is no more than 45 amino acids in length. In some embodiments, the at least one peptide ligand is no more than 50 amino acids in length.

In some embodiments, the at least one peptide ligand is from about 10 amino acids to about 100 amino acids in length. In some embodiments, the at least one peptide ligand is from about 10 amino acids to about 90 amino acids in length. In some embodiments, the at least one peptide ligand is from about 10 amino acids to about 80 amino acids in length. In some embodiments, the at least one peptide ligand is from about 10 amino acids to about 70 amino acids in length. In some embodiments, the at least one peptide ligand is from about 10 amino acids to about 60 amino acids in length. In some embodiments, the at least one peptide ligand is from about 10 amino acids to about 50 amino acids in length. In some embodiments, the at least one peptide ligand is from about 10 amino acids to about 40 amino acids in length. In some embodiments, the at least one peptide ligand is from about 10 amino acids to about 30 amino acids in length. In some embodiments, the at least one peptide ligand is from about 20 amino acids to about 100 amino acids in length. In some embodiments, the at least one peptide ligand is from about 30 amino acids to about 100 amino acids in length. In some embodiments, the at least one peptide ligand is from about 40 amino acids to about 100 amino acids in length. In some embodiments, the at least one peptide ligand is from about 50 amino acids to about 100 amino acids in length. In some embodiments, the at least one peptide ligand is from about 25 amino acids to about 50 amino acids in length.

In some embodiments, the at least one peptide ligand comprises an isoelectric point from about 3.5 to about 9.5. In some embodiments, the at least one peptide ligand comprises an isoelectric point from about 4.0 to about 9.0. In some embodiments, the at least one peptide ligand comprises an isoelectric point from about 5.0 to about 8.0. In some embodiments, the at least one peptide ligand comprises an isoelectric point from about 4.0 to about 7.0. In some embodiments, the at least one peptide ligand comprises an isoelectric point from about 6.0 to about 9.0.

In some embodiments, the at least one peptide ligand comprises a polarity value from about-1.2 to about 1.2. In some embodiments, the at least one peptide ligand exhibits a disassociation constant (K_{D}) less than or equal to about 1.0⁻⁴ M at pH 7.4. In some embodiments, the at least one peptide ligand exhibits a disassociation constant (K_{D}) less than or equal to about 10⁻⁵ M at a pH that is higher than or equal to 7.0. In some embodiments, the at least one peptide ligand exhibits a disassociation constant (K_{D}) higher than or equal to about 10⁻⁴ M at a pH that is lower than or equal to 6.5. In some embodiments, the at least one peptide ligand from (a) and/or (b) exhibits a dynamic binding capacity (DBC_{10%}) of at least 10¹³ vp/mL of resin.

In some embodiments, the at least one peptide ligand of the present disclosure from (a) comprises: (i) an amino acid sequence having at least 80% sequence identity with SEQ ID NO: 1; (ii) an amino acid sequence having at least 80% sequence identity with SEQ ID NO: 2; (iii) an amino acid sequence having at least 80% sequence identity with SEQ ID NO: 3; (iv) an amino acid sequence having at least 80% sequence identity with SEQ ID NO: 4; (v) an amino acid sequence having at least 80% sequence identity with SEQ ID NO: 5; (vi) an amino acid sequence having at least 80% sequence identity with SEQ ID NO: 6; (vii) an amino acid sequence having at least 80% sequence identity with SEQ ID NO: 7; (viii) an amino acid sequence having at least 80% sequence identity with SEQ ID NO: 8; (ix) an amino acid sequence having at least 80% sequence identity with SEQ ID NO: 9; (x) an amino acid sequence having at least 80% sequence identity with SEQ ID NO: 10; (xi) an amino acid sequence having at least 80% sequence identity with SEQ ID NO: 11; (xii) an amino acid sequence having at least 80% sequence identity with SEQ ID NO: 12; (xiii) an amino acid sequence having at least 80% sequence identity with SEQ ID NO: 13; (xiv) an amino acid sequence having at least 80% sequence identity with SEQ ID NO: 14; (xv) an amino acid sequence having at least 80% sequence identity with SEQ ID NO: 15; (xvi) an amino acid sequence having at least 80% sequence identity with SEQ ID NO: 16; (xvii) an amino acid sequence having at least 80% sequence identity with SEQ ID NO: 17; (xviii) an amino acid sequence having at least 80% sequence identity with SEQ ID NO: 33; (xix) an amino acid sequence having at least 80% sequence identity with SEQ ID NO: 34; and/or (xx) an amino acid sequence having at least 80% sequence identity with SEQ ID NO: 35.

In some embodiments, the at least one peptide ligand of the present disclosure from (a) comprises: (i) an amino acid sequence having at least 90% sequence identity with SEQ ID NO: 1; (ii) an amino acid sequence having at least 90% sequence identity with SEQ ID NO: 2; (iii) an amino acid sequence having at least 90% sequence identity with SEQ ID NO: 3; (iv) an amino acid sequence having at least 90% sequence identity with SEQ ID NO: 4; (v) an amino acid sequence having at least 90% sequence identity with SEQ ID NO: 5; (vi) an amino acid sequence having at least 90% sequence identity with SEQ ID NO: 6; (vii) an amino acid sequence having at least 90% sequence identity with SEQ ID NO: 7; (viii) an amino acid sequence having at least 90% sequence identity with SEQ ID NO: 8; (ix) an amino acid sequence having at least 90% sequence identity with SEQ ID NO: 9; (x) an amino acid sequence having at least 90% sequence identity with SEQ ID NO: 10; (xi) an amino acid sequence having at least 90% sequence identity with SEQ ID NO: 11; (xii) an amino acid sequence having at least 90% sequence identity with SEQ ID NO: 12; (xiii) an amino acid sequence having at least 90% sequence identity with SEQ ID NO: 13; (xiv) an amino acid sequence having at least 90% sequence identity with SEQ ID NO: 14; (xv) an amino acid sequence having at least 90% sequence identity with SEQ ID NO: 15; (xvi) an amino acid sequence having at least 90% sequence identity with SEQ ID NO: 16; (xvii) an amino acid sequence having at least 90% sequence identity with SEQ ID NO: 17; (xviii) an amino acid sequence having at least 90% sequence identity with SEQ ID NO: 33; (xix) an amino acid sequence having at least 90% sequence identity with SEQ ID NO: 34; and/or (xx) an amino acid sequence having at least 90% sequence identity with SEQ ID NO: 35.

In some embodiments, the at least one peptide ligand of the present disclosure from (a) comprises: (i) an amino acid sequence having at least 95% sequence identity with SEQ ID NO: 1; (ii) an amino acid sequence having at least 95% sequence identity with SEQ ID NO: 2; (iii) an amino acid sequence having at least 95% sequence identity with SEQ ID NO: 3; (iv) an amino acid sequence having at least 95% sequence identity with SEQ ID NO: 4; (v) an amino acid sequence having at least 95% sequence identity with SEQ ID NO: 5; (vi) an amino acid sequence having at least 95% sequence identity with SEQ ID NO: 6; (vii) an amino acid sequence having at least 95% sequence identity with SEQ ID NO: 7; (viii) an amino acid sequence having at least 95% sequence identity with SEQ ID NO: 8; (ix) an amino acid sequence having at least 95% sequence identity with SEQ ID NO: 9; (x) an amino acid sequence having at least 95% sequence identity with SEQ ID NO: 10; (xi) an amino acid sequence having at least 95% sequence identity with SEQ ID NO: 11; (xii) an amino acid sequence having at least 95% sequence identity with SEQ ID NO: 12; (xiii) an amino acid sequence having at least 95% sequence identity with SEQ ID NO: 13; (xiv) an amino acid sequence having at least 95% sequence identity with SEQ ID NO: 14; (xv) an amino acid sequence having at least 95% sequence identity with SEQ ID NO: 15; (xvi) an amino acid sequence having at least 95% sequence identity with SEQ ID NO: 16; (xvii) an amino acid sequence having at least 95% sequence identity with SEQ ID NO: 17; (xviii) an amino acid sequence having at least 95% sequence identity with SEQ ID NO: 33; (xix) an amino acid sequence having at least 95% sequence identity with SEQ ID NO: 34; and/or (xx) an amino acid sequence having at least 95% sequence identity with SEQ ID NO: 35.

In some embodiments, the at least one peptide ligand of the present disclosure from (a) comprises: (i) an amino acid sequence having at least 98% sequence identity with SEQ ID NO: 1; (ii) an amino acid sequence having at least 98% sequence identity with SEQ ID NO: 2; (iii) an amino acid sequence having at least 98% sequence identity with SEQ ID NO: 3; (iv) an amino acid sequence having at least 98% sequence identity with SEQ ID NO: 4; (v) an amino acid sequence having at least 98% sequence identity with SEQ ID NO: 5; (vi) an amino acid sequence having at least 98% sequence identity with SEQ ID NO: 6; (vii) an amino acid sequence having at least 98% sequence identity with SEQ ID NO: 7; (viii) an amino acid sequence having at least 98% sequence identity with SEQ ID NO: 8; (ix) an amino acid sequence having at least 98% sequence identity with SEQ ID NO: 9; (x) an amino acid sequence having at least 98% sequence identity with SEQ ID NO: 10; (xi) an amino acid sequence having at least 98% sequence identity with SEQ ID NO: 11; (xii) an amino acid sequence having at least 98% sequence identity with SEQ ID NO: 12; (xiii) an amino acid sequence having at least 98% sequence identity with SEQ ID NO: 13; (xiv) an amino acid sequence having at least 98% sequence identity with SEQ ID NO: 14; (xv) an amino acid sequence having at least 98% sequence identity with SEQ ID NO: 15; (xvi) an amino acid sequence having at least 98% sequence identity with SEQ ID NO: 16; (xvii) an amino acid sequence having at least 98% sequence identity with SEQ ID NO: 17; (xviii) an amino acid sequence having at least 98% sequence identity with SEQ ID NO: 33; (xix) an amino acid sequence having at least 98% sequence identity with SEQ ID NO: 34; and/or (xx) an amino acid sequence having at least 98% sequence identity with SEQ ID NO: 35.

In some embodiments, the composition comprises: (i) at least two peptide ligands from SEQ ID NOs: 1-17 and 33-35; (ii) at least three peptide ligands from SEQ ID NOs: 1-17 and 33-35; (iii) at least four peptide ligands from SEQ ID NOs: 1-17 and 33-35; (iv) at least five peptide ligands from SEQ ID NOs: 1-17 and 33-35; (v) at least six peptide ligands from SEQ ID NOs: 1-17 and 33-35; (vi) at least seven peptide ligands from SEQ ID NOs: 1-17 and 33-35; (vii) at least eight peptide ligands from SEQ ID NOs: 1-17 and 33-35; (viii) at least nine peptide ligands from SEQ ID NOs: 1-17 and 33-35; (ix) at least ten peptide ligands from SEQ ID NOs: 1-17 and 33-35; (x) at least eleven peptide ligands from SEQ ID NOs: 1-17 and 33-35; (xi) at least twelve peptide ligands from SEQ ID NOs: 1-17 and 33-35; (xii) at least thirteen peptide ligands from SEQ ID NOs: 1-17 and 33-35; (xiii) at least fourteen peptide ligands from SEQ ID NOs: 1-17 and 33-35; (xiv) at least fifteen peptide ligands from SEQ ID NOs: 1-17 and 33-35; (xv) at least sixteen peptide ligands from SEQ ID NOs: 1-17 and 33-35; (xvi) at least seventeen peptide ligands from SEQ ID NOs: 1-17 and 33-35; (xvii) at least eighteen peptide ligands from SEQ ID NOs: 1-17 and 33-35; (xviii) at least nineteen peptide ligands from SEQ ID NOs: 1-17 and 33-35; or (xix) all twenty peptide ligands of SEQ ID NOs: 1-17 and 33-35.

In accordance with these embodiments, the peptide ligands of the present disclosure can purify an AAV from a biological fluid. In some embodiments, the biological fluid is a cell culture fluid. In some embodiments, the biological fluid comprises a supernatant and/or a cellular lysate. In some embodiments, the biological fluid is derived from a virus production cell line. In some embodiments, the virus production cell line is selected from the group consisting of CHO cells, HEK293 cells, MDCK-S, MDCK-A, Vero cells, LLC-MK2D, PER.C6, EB66, AGE1.CR cells, Spodoptera frugiperda (Sf9) cells, and HeLa cells, or any derivatives or variants thereof.

b. Adsorbents

Embodiments of the present disclosure also include an adsorbent (e.g., adsorption chromatography) comprising any of the compositions described herein. Further described herein are adsorbents comprising a composition as described above, where each peptide ligand of the composition is conjugated to a support. Supports may comprise, but are not limited to, particles, beads, plastic surfaces, resins, fibers, and/or membranes. In some embodiments, the solid support comprises a non-porous or porous particle, a membrane, a plastic surface, a fiber or a woven or non-woven fibermat, a hydrogel, a microplate, and/or a microfluidic device. In some embodiments, the solid support comprises polymethacrylate and derivatives, polyolefin and derivatives, polyesters and derivatives, polyethers and derivatives, polystyrene, crosslinked polysaccharides, iron oxide, silica, titania, and/or zirconia. In some embodiments, supports may include microparticles and/or nanoparticles. Each support may be made out of any suitable material including, but not limited to, synthetic or natural polymers, metals, and metal oxides. Some supports may be magnetic, such as a magnetic bead, microparticle and/or nanoparticle. Suitable synthetic polymers include, but are not limited to, polymethacrylate, polyethersulfone, and polyethyleneglycole. Suitable natural polymers include, but are not limited to, cellulose, crosslinked agarose, and chitosan. Suitable metal oxides include, but are not limited to, iron oxide, silica, titania, and zirconia. Further described herein are adsorbents comprising a composition as described above conjugated to a support.

In some embodiments, the adsorbent comprises a single type of support made from a single type of support material, where all of the peptides in the composition are conjugated to supports formed of the single type of support material. In these embodiments, the composition may comprise one or more different types of peptides, each conjugated to the single type of support made from the single type of support material. In other embodiments, the adsorbent comprises a plurality of types of support. Each type of support may be made of the same type of support material or different types of support materials. In these embodiments, the composition may comprise one or more different types of peptides, as described further herein, each conjugated to a different type of support. In still other embodiments, the peptides of the composition can be conjugated to a soluble compound, for example stimuli-responsive polymer chains to remove AAVs by affinity precipitation.

c. Methods

As described further herein, the present disclosure also provides improved methods for purifying an AAV from a biological fluid comprising one or more product- and/or process-related impurities or contaminants, as compared to currently used methods. In some embodiments, the method includes contacting a composition comprising any of the peptide ligands described herein, or an adsorbent comprising any of the peptides described herein, with a biological fluid comprising the AAV, wherein the at least one peptide ligand binds the AAV. In accordance with these embodiments, the method includes eluting the AAV from the peptide ligand, thereby purifying the AAV. The methods of the present disclosure can further comprise washing the composition or adsorbent to remove one or more product- and/or process-related impurities or contaminants from the AAVs bound to the peptide ligands. In some embodiments, the method can be performed under any binding conditions suitable for use with the composition or adsorbent, including both static binding conditions and dynamic binding conditions.

As described further herein, the peptide ligands of the present disclosure exhibit unique operational features that provide distinct technological advantages over the compositions and methods currently available to purify AAVs. For example, currently available ligands that are used to purify AAVs require harsh elution conditions (e.g., pH<3.0), which damages the AAV products being eluted. In contrast, the peptide ligands of the present disclosure release bound AAVs under much gentler conditions, which do not damage the AAV product. In accordance with this, embodiments of the present disclosure include compositions comprising at least one peptide ligand described herein that can be used to elute AAVs at a pH ranging from about 6.0 to about 7.5. In some embodiments, a composition comprising at least one peptide ligand described herein, or an adsorbent comprising at least one peptide ligand described herein, is substantially resistant to NaOH, or a similar alkaline agent, at a concentration from about 0.1M to about 0.5M. In some embodiments, a composition comprising at least one peptide ligand described herein, or an adsorbent comprising at least one peptide ligand described herein, is substantially reusable for up to about 10 cycles. In some embodiments, the method further comprises a washing step before eluting the AAV from the at least one peptide ligand. As described further herein, the AAV purification methods of the present disclosure result in at least a 50% yield for the AAV. In some embodiments, the method produces at least an 80-fold reduction in host cell proteins.

The binding affinity of the compositions and/or adsorbent for the AAVs, as compared to one or more product- and/or process-related impurities or contaminants, can be adjusted by changes in the following: properties and concentration of the AAVs, properties and concentration of the one or more product- and/or process-related impurities or contaminants; the properties and concentration of the host cell proteins; the composition, concentration, and pH of the mixture; the loading conditions and residence time of the contacting and washing steps; and/or the composition, concentration, and pH of the aqueous buffers utilized to conduct the purification of the AAV from the mixture using the compositions and/or adsorbent. Any of these variables can be changed to variables which are suitable according to the methods of the present disclosure and result in increased or decreased binding affinity as required for the present disclosure.

In some embodiments, the contacting step can comprise a low pH buffer of between pH 5-9. In some embodiments, the contacting step can comprise a low pH buffer of between pH 5-8. In some embodiments, the contacting step can comprise a low pH buffer of between pH 5-7. In some embodiments, the contacting step can comprise a low pH buffer of between pH 6-9. In some embodiments, the contacting step can comprise a low pH buffer of between pH 6-8. In some embodiments, the contacting step can comprise a low pH buffer of between pH 6-7. In some embodiments, the contacting step can comprise a low pH buffer of between pH 7-9. In some embodiments, the contacting step can comprise a low pH buffer of between pH 7-8.

In some embodiments, the elution is performed at pH from about 5.0 to about 8.0. In some embodiments, the elution is performed at pH from about 5.0 to about 7.5. In some embodiments, the elution is performed at pH from about 5.0 to about 7.0. In some embodiments, the elution is performed at pH from about 5.0 to about 6.5. In some embodiments, the elution is performed at pH from about 5.0 to about 6.0. In some embodiments, the elution is performed at pH from about 5.0 to about 5.5. In some embodiments, the elution is performed at pH from about 5.5 to about 8.0. In some embodiments, the elution is performed at pH from about 6.0 to about 8.0. In some embodiments, the elution is performed at pH from about 6.5 to about 8.0. In some embodiments, the elution is performed at pH from about 7.0 to about 8.0. In some embodiments, the elution is performed at pH from about 7.5 to about 8.0. In some embodiments, the elution is performed at pH from about 6.0 to about 7.0. In some embodiments, the elution is performed at pH from about 5.5 to about 7.5.

In some embodiments, the methods of the present disclosure result in at least a 50% yield for the AAV (e.g., as compared to methods in which the peptide ligands of the present disclosure are not used). In some embodiments, the methods of the present disclosure result in at least a 60% yield for the AAV. In some embodiments, the methods of the present disclosure result in at least a 70% yield for the AAV. In some embodiments, the methods of the present disclosure result in at least an 80% yield for the AAV. In some embodiments, the methods of the present disclosure result in at least a 90% yield for the AAV.

In some embodiments, the methods of the present disclosure produce at least an 80-fold reduction in host cell proteins when purifying an AAV (e.g., as compared to methods in which the peptide ligands of the present disclosure are not used). In some embodiments, the methods of the present disclosure produce at least a 100-fold reduction in host cell proteins. In some embodiments, the methods of the present disclosure produce at least a 150-fold reduction in host cell proteins. In some embodiments, the methods of the present disclosure produce at least a 200-fold reduction in host cell proteins. In some embodiments, the methods of the present disclosure produce at least a 250-fold reduction in host cell proteins. In some embodiments, the methods of the present disclosure produce at least a 300-fold reduction in host cell proteins. In some embodiments, the methods of the present disclosure produce at least a 350-fold reduction in host cell proteins. In some embodiments, the methods of the present disclosure produce at least a 400-fold reduction in host cell proteins.

Embodiments of the present disclosure also include an adeno-associated virus (AAV) purified using any of the methods described herein. In some embodiments, the AAV exhibits at least 25% transduction efficiency (i.e., a measure of biological activity). In some embodiments, the AAV exhibits at least 30% transduction efficiency. In some embodiments, the AAV exhibits at least 35% transduction efficiency. In some embodiments, the AAV exhibits at least 40% transduction activity. In some embodiments, the AAV exhibits at least 45% transduction efficiency. In some embodiments, the AAV exhibits at least 50% transduction efficiency. In some embodiments, the AAV exhibits at least 55% transduction efficiency. In some embodiments, the AAV exhibits at least 60% transduction efficiency. In some embodiments, the AAV exhibits at least 65% transduction efficiency. In some embodiments, the AAV exhibits at least 70% transduction efficiency. In some embodiments, the AAV exhibits at least 75% transduction efficiency. In some embodiments, the AAV exhibits at least 80% transduction efficiency. In some embodiments, the AAV exhibits at least 85% transduction efficiency. In some embodiments, the AAV exhibits at least 90% transduction efficiency. In some embodiments, the AAV exhibits at least 95% transduction efficiency.

### 3. Materials and Methods

In silico design of peptide mimetics of the AAV receptor (AAVR) and anti-AAV antibody A20. The crystal structures of AAVR in complex with AAV1 (PDB ID: 6JCQ and 7T15), AAV2 (3J1S, 6IHB and 6NZ0), AAV5 (7KP3 and 7KPN), and AAV9 (7WJX and 7WQP) as well as the complex of AAV2 with monoclonal antibody A20 (3J1S) were analyzed to identify the residues on the protein ligands (AAVR and A20) and the AAV virion protein (VP1) involved in the affinity interaction and calculate their pairwise contributions to the binding energy. Based on this analysis, the A20-mimetic candidates CYGHFSGYGNYGPC (SEQ ID NO: 4), CYGHFSPYGNYGPC (SEQ ID NO: 5), CYHFSYNYPC (SEQ ID NO: 9), CYHFSYNYPKSC (SEQ ID NO: 8), CYIHFSGYTNYNGSLKSC (SEQ ID NO: 2), CYIHFSGYTNYNPC (SEQ ID NO: 6), CYIHFSGYTNYNPSLKSC (SEQ ID NO: 1), CYIHFSPYTNYNPSLKSC (SEQ ID NO: 7), CYVHFSGYSNYSPSC (SEQ ID NO: 3), GCGQQYWIGPFTFGCG (SEQ ID NO: 22), GQQYWIGPFTFG (SEQ ID NO: 23), LETVKPGLYEPITHPRDYS (SEQ ID NO: 24), and SYDRPHTIPEYLGPKVTEL (SEQ ID NO: 25); and the AAVR-mimetic candidates AIVSPQFQEISLPTTSTVIDGSQSTDDDKIVQY (SEQ ID NO: 26), CDGSQSTDDDKIC (SEQ ID NO: 11), CDSQSTDDDKIC (SEQ ID NO: 12), CSGSTDDDKIC (SEQ ID NO: 13), CSGSTEQEKIC (SEQ ID NO: 27), CVIDGSQSTDDDKIC (SEQ ID NO: 10), CVIDGSQSTDDDKIVQYC (SEQ ID NO: 28), GCLITHPRDYS (SEQ ID NO: 29), GCLITHPRDYSGCG (SEQ ID NO: 30), GYIHFSGYTNYNPSLKS (SEQ ID NO: 31), GYWIGPFTGGGYIHFSGYT (SEQ ID NO: 14), GYWIGPFTGPGYIHFSGYT (SEQ ID NO: 15), GYWIGPFTPGPYIHFSGYT (SEQ ID NO: 16), LITHPRDYSPKLTPGLYEFG (SEQ ID NO: 17), and TVIDGSQSTDDDKIVQY (SEQ ID NO: 32) were constructed using the molecular editor Avogadro and their structures were prepared in GROMACS using the force field GROMOS 54A7; disulfide-cyclic peptide sequences A1-A12 were designed in the cyclic format GC-X₁X₂[ . . . ]Xₙ—C-GSG, whereas linear peptide sequences A12-A16 were designed in the linear format G-X₁X₂[ . . . ]Xₙ-GSG. Each peptide sequence was placed in a simulation box with periodic boundary containing 1,500 TIP3P water molecules and equilibrated with 10,000 steps of steepest gradient descent; heated to 300 K in an NVT ensemble for 250 ps using 1 fs time steps; and equilibrated to 1 atm via a 500-ps NPT simulation with 2 fs time steps. The production runs were conducted in the NPT ensemble under constant 300 K and 1 atm by applying the Nosé-Hoover thermostat and the Parrinello-Rahman barostat, respectively; the motion equations were integrated using the leap-frog algorithm with steps of 2 fs; covalent bonds were constrained using the LINCS algorithm; Lennard-Jones and short-range electrostatic interactions were calculated using cut-off values of 0.8 nm and 1.2 nm, respectively; the particle-mesh Ewald method was implemented for the long-range electrostatic interactions; the lists of bonded and non-bonded interactions (cutoff of 1.2 nm) were updated every 2 and 6 fs, respectively. The energetic landscape associated with the various peptide conformations was sampled to identify the structures with absolute energy minima. The structure of the VP1 from AAV1 (PDB ID: 6JCR), AAV2 (6IH9), AAV3 (3KIC), AAV4 (2G8G), AAV5 (7KP3), AAV6 (5EGC), AAV7 (7JOT), AAV8 (2QA0), and AAV9 (7WJX) were initially prepared using Protein Prep Wizard (PPW, Schrödinger, New York, NY) by correcting missing residues or atoms, adding explicit hydrogens, removing salt ions, and optimizing the hydrogen-bonding network. The resulting VPs were utilized to construct triangular clusters of VP1-VP2-VP3 proteins, whose ionization states at pH 6.0 and 7.4 were obtained—and the corresponding structural minimization of the clusters were performed using PROPKA. The construct triangular clusters were finally draped on the spherical cap of the corresponding AAV capsid. The peptide ligands were then docked in silico against the AAVR binding sites using the docking software HADDOCK (High Ambiguity Driven Protein-Protein Docking) v.2.4. The AAVR-binding residues and the A20-binding residues on the VP proteins and residues X₁X₂[ . . . ]Xₙ on the peptides were denoted as “active”, while all surrounding residues were marked as “passive”. Clusters of up to 20 docked AAV: peptide structures selected based on Cα RMSD<7.5 Å were ranked using the dMM-PBSA score. Finally, the top AAV: peptide complexes were refined via 200-ns MD simulations to estimate the free energy of binding (AGB).

Materials. Fluorenylmethoxycarbonyl-(Fmoc-) protected amino acids Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, and Fmoc-Val-OH, the coupling agent Hexafluorophosphate Azabenzotriazole Tetramethyl Uronium (HATU), diisopropylethylamine (DIPEA), piperidine, and trifluoroacetic acid (TFA) were procured from ChemImpex International (Wood Dale, IL, USA). The Toyopearl NH2-750F resin (pore size >100 nm; particle size: 45 μm; ligand density: 200 μmol per mL resin) was obtained from Tosoh Bioscience (Tokyo, Japan). Triisopropylsilane (TIPS), Kaiser test kits, 1,2-ethanedithiol (EDT), polybrene, and phosphate buffered saline (PBS) tablets were from MilliporeSigma (St. Louis, MO, USA). Dichloromethane (DCM), methanol, N-methyl-2-pyrrolidone (NMP), N,N′-dimethylformamide (DMF), Bis-Tris HCl, magnesium chloride (MgCl₂), phosphoric acid, potassium chloride (KCl), sodium chloride (NaCl), sodium hydroxide (NaOH), Pluronic™ F-68, POROS™ CaptureSelect™ AAVX Affinity Resin, and SilverQuest™ Silver Staining Kit were obtained from Fisher Chemical (Hampton, NH, USA). The AVB Sepharose HP was sourced from Cytiva (Marlborough, MA). Dulbecco's Modified Eagle Medium (DMEM) and fetal bovine serum (FBS), Gibco™ Viral Production Cells 2.0, AAV-MAX Enhancer, Viral-Plex™ Complexation Buffer, Transfection Reagent, benzonase endonuclease, and AAV-MAX Lysis Buffer were obtained from ThermoFisher Scientific (Waltham, MA). Human fibrosarcoma (HT1080) cells were sourced from ATCC (Manassas, VA). Pure AAV2, AAV6, AAV8, and AAV9 were sourced from Charles River Laboratories (Durham, NC). The Alltech chromatography columns (diameter: 3.6 mm; length: 50 mm; volume: 0.5 mL), and 10 μm polyethylene frits were obtained from VWR International (Radnor, PA, USA). The AAV ELISA kits were purchased from Progen (Wayne, PA, USA) while the HEK293 ELISA kits were purchased from Cygnus (Southport, NC, USA). The BioResolve SEC mAb Column (particle diameter: 2.5 μm; pore diameter: 200 Å; column diameter: 7.8 mm; column length: 300 mm) size exclusion chromatography column was from Waters Inc. (Milford, MA, USA). The CIMac PrimaS™ 0.1 mL analytical monolith column (diameter: 5.2 mm; length: 4.95 mm; volume: 0.1 mL, channel radius: 1050 nm) for steric exclusion chromatography analysis was obtained from BIA separations (Ajdovscina, Slovenia). The 10-20% Tris-Glycine HCl SDS-PAGE gels were purchased from Bio Rad Life Sciences (Hercules, CA, USA). In house production of AAV6, AAV8, and AAV9 were conducted using plasmids from Aldevron; plasmids pAAV2/9n, pAAV2/8, and pAdDeltaF6 were a gift from James M. Wilson (Addgene plasmid #112865, #112864, and #112867, respectively); pDGM6 was a gift from David Russell (Addgene plasmid #110660);⁶⁶ pAAV CAGG eGFP was a gift from Troy Margrie (Addgene plasmid #107707).

Synthesis of peptide ligands on Toyopearl resin. Sequences CYIHFSGYTNYNPSLKSC (A1; SEQ ID NO: 1), CYIHFSGYTNYNGSLKSC (A2; SEQ ID NO: 2), CYVHFSGYSNYSPSC (A3; SEQ ID NO: 3), CYGHFSGYGNYGPC (A4; SEQ ID NO: 4), CYGHFSPYGNYGPC (A5; SEQ ID NO: 5), CYIHFSGYTNYNPC (A6; SEQ ID NO: 6), CYIHFSPYTNYNPC (A7; SEQ ID NO: 7), CYHFSYNYPKSC (A8; SEQ ID NO: 8), CYHFSYNYPC (A9; SEQ ID NO: 9), CVIDGSQSTDDDKIC (A10; SEQ ID NO: 10), CDGSQSTDDDKIC (A11; SEQ ID NO: 11), CDSQSTDDDKIC (A12; SEQ ID NO: 12), CSGSTDDDKIC (A13; SEQ ID NO: 13), GYWIGPFTGGGYIHFSGYT (A14; SEQ ID NO: 14), GYWIGPFTGPGYIHFSGYT (A15; SEQ ID NO: 15), GYWIGPFTPGPYIHFSGYT (A16; SEQ ID NO: 16), and LITHPRDYSPKLTPGLYEFG (A17; SEQ ID NO: 17) were synthesized on Toyopearl NH2-750F (TP750F) resin using an Initiator+ Alstra™ automated peptide synthesizer (Biotage, Uppsala, Sweden). Each amino acid coupling step was performed using 3 equivalents (eq.) of Fmoc/tBu-protected amino acid, 3 eq. of HATU and 0.5 M, 6 eq. of DIPEA—all at the concentration of 0.5 M—in dry DMF at 45° C. for 20 min. The yield of all amino acid coupling steps was monitored via Kaiser test, while the removal of Fmoc groups was performed using 20% v/v piperidine in DMF at room temperature for 30 min. The final peptide density varied within the range of 0.11-0.16 mmol per gram of resin. Following chain elongation, the peptides were deprotected via acidolysis using a cleavage cocktail containing TFA, thioanisole, anisole, and EDT (94/3/2/1) for 2 hrs. After deprotection, the peptide-TP750F resins were washed sequentially with DCM, DMF, methanol, and stored in 20% v/v aqueous methanol.

Production and harvest of AAV2 from HEK293 cell cultures. Gibco™ Viral Production Cells 2.0 were initially diluted to 3.0×10⁶ cells/mL incubated with AAV-MAX Enhancer at 1% v/v in a humidified incubator with 8% CO₂ while shaken at 120 rpm at 37° C. The transfection cocktail was prepared by combining the pRC2 plasmid, which contains the REP and CAP genes for AAV2, the pHelper plasmid, which contains helper genes necessary for AAV replication, and the pAAV-GFP plasmid, which containing the GFP gene to be packaged into the newly formed AAVs, at the molar DNA ratio of 1:1:1 (pRC2: pHelper: pAAV-GFP) and the total concentration of 1.5 μg per mL of culture. The plasmid cocktail was diluted in Viral-Plex™ Complexation Buffer to 10% of the culture volume. In parallel, volumes of Transfection Booster and Transfection Reagent corresponding to 0.3% and 0.6% of the culture volume were mixed. The DNA/Viral-Plex and the Transfection Booster/Transfection Reagent components were incubated at room temperature for 10 minutes, mixed, and incubated with the Gibco™ Viral Production Cells at room temperature for 20 minutes, after which the cells were returned to the humidified incubator. After 72 hours, AAV-MAX Lysis Buffer was added at a volume corresponding to 10% of the culture volume to lyse cells. The cell lysate was subsequently added with 2 mM MgCl₂ and 90 U/mL benzonase endonuclease (GENIUS™ Nuclease) and incubated at 37° C. for 2 hours. The lysate was then clarified via centrifugation at 4100 g for 40 minutes and the resulting supernatant was collected.

Preparation of feed samples. Clarified HEK293 cell lysates containing AAV1 and AAV5 were obtained from UNC Vector core (Chapel Hill, NC); clarified HEK293 cell lysates containing AAV2, AAV6, AAV8, and AAV9 were prepared as described herein, while the clarified Sf9 cell lysate containing AAV2 was obtained from BTEC (Raleigh, NC). Purified AAV1, AAV2, AAV5, AAV6, AAV8, and AAV9 were prepared at the titer of ~5.0 10¹¹-5.0 10¹² vp/mL in 20 mM NaCl in 10 mM Bis-Tris at pH 7.0. The cell culture lysates were diafiltered against 20 mM NaCl in 10 mM Bis-Tris at pH 7.0 to achieve a final AAV2 titer of ~1.9 10¹² vp/mL, and an HCP titer of ~0.3 mg/mL in the HEK293 harvests and ~1.1 mg/mL in the Sf9 harvest.

Binding studies of AAV1, AAV2, AAV5, AAV6, AAV8, and AAV9 in non-competitive conditions using peptide-TP750F resins. A volume of 0.5 mL of POROS™ CaptureSelect™ AAVX affinity resin, AVB Sepharose HP resin, (A1; SEQ ID NO: 1) CYIHFSGYTNYNPSLKSC-, (A2; SEQ ID NO: 2) CYIHFSGYTNYNGSLKSC-, (A3; SEQ ID NO: 3) CYVHFSGYSNYSPSC-, (A4; SEQ ID NO: 4) CYGHFSGYGNYGPC-, (A5; SEQ ID NO: 5) CYGHFSPYGNYGPC-, (A6; SEQ ID NO: 6) CYIHFSGYTNYNPC-, (A7; SEQ ID NO: 7) CYIHFSPYTNYNPC-, (A8; SEQ ID NO: 8) CYHFSYNYPKSC—, (A9; SEQ ID NO: 9) CYHFSYNYPC—, (A10; SEQ ID NO: 10) CVIDGSQSTDDDKIC-, (A11; SEQ ID NO: 11) CDGSQSTDDDKIC-, (A12; SEQ ID NO: 12) CDSQSTDDDKIC-, (A13; SEQ ID NO: 13) CSGSTDDDKIC-, (A14; SEQ ID NO: 14; SEQ ID NO: 14) GYWIGPFTGGGYIHFSGYT-, (A15; SEQ ID NO: 15) GYWIGPFTGPGYIHFSGYT-, (A16; SEQ ID NO: 16) GYWIGPFTPGPYIHFSGYT-, and (A17; SEQ ID NO: 17) LITHPRDYSPKLTPGLYEFG-TP750F resins was initially packed in an Alltech chromatography column and washed with 10 column volumes (CVs) of 20% v/v ethanol, 10 CVs of MilliQ water, and 10 CVs of binding buffer (10 mM Bis-Tris, 20 mM NaCl buffer at pH 7.0). A volume of 10 mL of pure AAV solution in binding buffer was loaded on the column at the flow rate of 0.17 mL/min (residence time, RT: 3 min). The resin was washed with 20 CVs of binding buffer at 0.5 mL/min. The bound AAVs were eluted from the peptide-TP750F resins using 1 M MgCl₂ in 10 mM Bis-Tris HCl buffer at pH 6.0 at the flow rate of 0.5 mL/min (RT: 1 min); and from POROS™ CaptureSelect™ AAVX affinity resin and AVB Sepharose HP resin respectively using 0.2 M MgCl₂ in 200 mM citrate buffer at pH 2.2 and PBS at pH 2.0 at the flow rate of 0.25 mL/min (RT: 2 min). All resins were regenerated with 10 CVs of phosphate buffered saline at pH 2.0 at the flow rate of 0.5 mL/min. All dynamic binding experiments were performed using a ÄKTA Avant system from Cytiva (Marlborough, MA, USA), while continuously monitoring the effluent stream via UV spectrometry at 280 nm. The collected fractions were analyzed using serotype-specific AAV ELISA Kit as described herein to quantify the values of AAV-binding and recovery.

Dynamic AAV2 binding capacity of peptide-TP750F resins. A volume of 0.5 mL of POROS™ CaptureSelect™ AAVX affinity resin, AVB Sepharose HP resin, (A1; SEQ ID NO: 1) CYIHFSGYTNYNPSLKSC-TP750F resin, (A4; SEQ ID NO: 4) CYGHFSGYGNYGPC-, and (A10; SEQ ID NO: 10) CVIDGSQSTDDDKIC-TP750F resins were initially packed and equilibrated with binding buffer as described herein. A volume of 45 mL of HEK293 cells lysate containing AAV2 and diafiltered into binding buffer was loaded on the column at the flow rate of 0.17 mL/min (residence time, RT: 3 min). Resin washing and elution of bound AAV2 from the peptide-TP750F resins and the commercial resins was performed as described herein. All dynamic binding experiments were performed using a ÄKTA Avant system from Cytiva (Marlborough, MA, USA), while continuously monitoring the effluent stream via UV spectrometry at 280 nm. The collected fractions were analyzed by AAV2 ELISA Kit as described herein to generate the breakthrough curves, and the resulting chromatograms were utilized to calculate the DBC_{10%}.

Purification of AAV2 from clarified HEK293 and Sf9 cell lysate using peptide-TP750F resins. A volume of 0.5 mL of POROS™ CaptureSelect™ AAVX affinity resin, AVB Sepharose HP resin, (A1; SEQ ID NO: 1) CYIHFSGYTNYNPSLKSC-, (A3; SEQ ID NO: 3) CYVHFSGYSNYSPSC-, (A4; SEQ ID NO: 4) CYGHFSGYGNYGPC-, (A6; SEQ ID NO: 6) CYIHFSGYTNYNPC-, (A10; SEQ ID NO: 10) CVIDGSQSTDDDKIC-, and (A12; SEQ ID NO: 12) CDSQSTDDDKIC-TP750F resins were initially packed and equilibrated with binding buffer as described herein. A volume of 10 mL of AAV2 at ~1.9·10¹² vp/mL in HEK293 cell culture lysate (HCP titer ~0.3 mg/mL) was loaded at the flow rate of 0.17 mL/min (RT: 3 min). Resin washing and elution of bound AAV2 from the peptide-TP750F resins and the commercial resins was performed as described herein. All resins were regenerated with 10 CVs of PBS buffer at pH 2.0 at the flow rate of 0.5 mL/min and re-used for 20 additional cycles of AAV2 purification. The collected flow-through and elution fractions were analyzed by ELISA Kit to determine the values of AAV2 yield; HEK293 and Sf9 ELISA Kits were used to quantify the values of HCP removal; size exclusion chromatography and steric exclusion chromatography were used to measure global product purity; transmission electron microscopy was used to evaluate the integrity of the eluted AAVs; and fluorescence flow cytometry was used to quantify the transduction efficiency of the eluted AAVs.

Capsid quantification via serotype-specific AAV ELISA kits. The AAV titer in the feed, flow-through, and elution samples collected as described herein was measured using AAV Titration ELISA kit (PROGEN, Wayne, PA) following the manufacturer's protocol.

Quantification of HCPs. The titer of HEK293 and Sf9 HCPs in the feed, flow-through and elution samples collected as described herein was measured using a Generation 3 HEK293 HCP ELISA kit and a Generation 2 Sf9 HCP ELISA kit (Cygnus Technologies, Southport, NC) following the manufacturer's protocols.

Analytical size-exclusion chromatography (SEC). The feed, flow-through, and elution samples collected as described herein were analyzed by SEC HPLC using a BioResolve SEC column (Waters, Milford, MA) operated with a 40-min isocratic method using PBS at pH 7.0 (0.05% v/v sodium azide) at the flow rate of 0.50 mL/min. A volume of 10 μL of sample was injected and the effluent continuously monitored via UV (abs: 260 nm and 280 nm) fluorescence spectroscopy (ex/em: 280/350 nm).

Analytical steric-exclusion chromatography (SXC). The feed, flow-through, and elution samples collected as described herein were analyzed via analytical SXC using a monolith 0.1 mL CIMac PrimaS™ analytical column (BIA Separations, Slovenia) operated with a 20-min linear gradient from 100:0 A:B to 0:100 A:B (mobile phase A: 10% v/v PEG 6K in PBS at pH 7.0; mobile phase B: 3×PBS at pH 7.0) at the flow rate of 0.33 mL/min. Injection volumes were normalized based on the AAV titer measured via ELISA kits as described herein. The effluent continuously monitored via fluorescence spectroscopy (ex/em: 280/350 nm).

AAV imaging via transmission electron microscopy (TEM). The sample grids were glow discharged using the Pelco easiGlow™ unit and taken to a biosafety cabinet, where 3 μL of sample was added on the grid and incubated for 60 seconds at room temperature. Each sample was then blotted using a Whatman paper, and 5 μL of MilliQ water was added on the grid and blotted for 3 times. Finally, 3 μL of 1% v/v Nano-W stain was added on the grid, incubated for 5 seconds, and blotted. The sample grid was dried in a desiccator for 15 minutes and placed in a clean sponge box overnight. The sample grids were imaged using a miniTEM™ system (Vironova, Stockholm, Sweden) using the VIAS software for image collection and analysis.

Fluorescence flow cytometry (FFC). HT1080 cells were cultured in DMEM media supplemented with 10% v/v FBS at 5% CO₂ and 37° C. until reaching 80-90% confluence. Cells were then seeded in 96-well plates at a density of 6,000 cells/well and cultured overnight. The AAV2 in the HEK293 cell lysate and the eluted samples were serially diluted in DMEM (no FBS and antibiotics) added with polybrene at 8 μg/mL. A volume of 0.1 mL of diluted AAV sample was incubated with the HT1080 cells. After 24 hrs, spent medium was replaced with fresh DMEM supplemented with 10% v/v FBS and the cells were cultured for 72 hrs. The fraction of cells expressing GFP (GFP+) was quantified using a CytoFlex flow cytometer (Beckman Coulter, Brea, CA) and the number of transduction units per mL (TU/mL) was calculated using Equation 1:

$$Activity ( \frac{TU}{mL} ) = \frac{{N}_{HT 1080} \times \% {GFP}^{+}}{V \times DF} \qquad Equation 1$$

Wherein N_{HT1080} is the number of cells incubated with the diluted AAV sample, V is the volume of the diluted AAV sample, and DF is the dilution factor.

Materials. Fluorenylmethoxycarbonyl-(Fmoc-) protected amino acids Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, and Fmoc-Val-OH, the coupling agent Hexafluorophosphate Azabenzotriazole Tetramethyl Uronium (HATU), diisopropylethylamine (DIPEA), piperidine, and trifluoroacetic acid (TFA) were procured from ChemImpex International (Wood Dale, IL, USA). The Toyopearl NH2-750F resin (pore size >100 nm; particle size: 45 μm; ligand density: 200 μmol per mL resin) was obtained from Tosoh Bioscience (Tokyo, Japan). Triisopropylsilane (TIPS), Kaiser test kits, 1,2-ethanedithiol (EDT), polybrene, and phosphate buffered saline (PBS) tablets were from MilliporeSigma (St. Louis, MO, USA). Dichloromethane (DCM), methanol, N-methyl-2-pyrrolidone (NMP), N,N′-dimethylformamide (DMF), Bis-Tris HCl, magnesium chloride (MgCl₂), phosphoric acid, potassium chloride (KCl), sodium chloride (NaCl), sodium hydroxide (NaOH), Pluronic™ F-68, Poros™ CaptureSelect™ AAVX Affinity Resin, and SilverQuest™ Silver Staining Kit were obtained from Fisher Chemical (Hampton, NH, USA). The AVB Sepharose HP was sourced from Cytiva (Marlborough, MA). Dulbecco's Modified Eagle Medium (DMEM) and fetal bovine serum (FBS), Gibco™ Viral Production Cells 2.0, AAV-MAX Enhancer, Viral-Plex™ Complexation Buffer, Transfection Reagent, benzonase endonuclease, EXPRESS qPCR SuperMix with Premixed ROX (A10313), and AAV-MAX Lysis Buffer were obtained from ThermoFisher Scientific (Waltham, MA). Human fibrosarcoma (HT1080) cells were sourced from ATCC (Manassas, VA). Pure AAV2, AAV6, AAV8, and AAV9 were sourced from Charles River Laboratories (Durham, NC). The Alltech chromatography columns (diameter: 3.6 mm; length: 50 mm; volume: 0.5 mL), and 10 μm polyethylene frits were obtained from VWR International (Radnor, PA, USA). The AAV ELISA kits were purchased from Progen (Wayne, PA, USA) while the HEK293 ELISA kits were purchased from Cygnus (Southport, NC, USA). The BioResolve SEC mAb Column (particle diameter: 2.5 μm; pore diameter: 200 Å; column diameter: 7.8 mm; column length: 300 mm) size exclusion chromatography column was from Waters Inc. (Milford, MA, USA). The CIMac™ OH 0.1 mL analytical monolith column (diameter: 5.2 mm; length: 4.95 mm; volume: 0.1 mL, channel radius: 1050 nm) for steric exclusion chromatography analysis was obtained from BIA separations (Ajdovscina, Slovenia). The 10-20% Tris-Glycine HCl SDS-PAGE gels and ddPCR™ Supermix for Probes were purchased from Bio Rad Life Sciences (Hercules, CA, USA). Plasmids pAAV2/9n (Addgene plasmid #112865), pAAV2/7 ((Addgene plasmid #112863), pAdDeltaF6 (Addgene plasmid #112867), pAAV2/8 (Addgene plasmid #112864) were gifted by James M. Wilson; plasmids pDGM6 (Addgene plasmid #110660) and pDGM3B (Addgene plasmid #110809) were gifted by David Russell; finally, plasmid pAAV CAGG eGFP was a gift from Troy Margrie (Addgene plasmid #107707).

Conjugation of peptide ligands on chromatographic resins. Peptides CYIHFSGYTNYNPSLKSC (A1; SEQ ID NO: 1), CYVHFSGYSNYSPSC (A3; SEQ ID NO: 3), CYGHFSGYGNYGPC (A4; SEQ ID NO: 4), CYIHFSGYTNYNPC (A6; SEQ ID NO: 6), CVIDGSQSTDDDKIC (A10; SEQ ID NO: 10), CVIDGSASTDDDRIC (A10′; SEQ ID NO: 33), CVIDGSSSTDDDRIC (A10″; SEQ ID NO: 34), and CVIDGSASTDDDHIC (A10′″; SEQ ID NO: 35), CDGSQSTDDDKIC (A11; SEQ ID NO: 11), CDSQSTDDDKIC (A12; SEQ ID NO: 12), and CSGSTD-DDKIC (A13; SEQ ID NO: 13) were synthesized on Toyopearl NH2-750F (henceforth, Toyopearl) resin using an Initiator+ Alstra™ automated peptide synthesizer (Biotage, Uppsala, Sweden). Each amino acid coupling step was performed using 3 equivalents (eq.) of Fmoc/tBu-protected amino acid, 3 eq. of HATU and 0.5 M, 6 eq. of DIPEA—all at the concentration of 0.5 M in dry DMF at 45° C. for 20 min. The yield of all amino acid coupling steps was monitored via Kaiser test, while the removal of Fmoc groups was performed using 20% v/v piperidine in DMF at room temperature for 30 min. The final peptide density varied within the range of 0.11-0.16 mmol per gram of resin. Following chain elongation, the peptides were deprotected via acidolysis using a cleavage cocktail containing TFA, thioanisole, anisole, and EDT (94/3/2/1) for 2 hrs. After deprotection, the peptide-Toyopearl resins were washed sequentially with DCM, DMF, methanol, and stored in 20% v/v aqueous methanol.

Production and harvest of AAV from HEK293 cell cultures. Gibco™ Viral Production Cells 2.0 were initially diluted to 3·10⁶ cells/mL incubated with AAV-MAX Enhancer at 1% v/v in a humidified incubator with 8% CO₂ while shaken at 120 rpm at 37° C. The transfection cocktail was prepared by combining the pRC2 plasmid, which contains the Rep and Cap genes for each AAV serotype, the pHelper plasmid, which contains helper genes necessary for AAV replication, and the pAAV-GFP plasmid, which contains the GFP gene to be packaged into the newly formed AAVs, at the molar ratios listed in Table 1 and a total concentration of 1.5 μg DNA per mL of culture. The plasmid cocktail was diluted in Viral-Plex™ Complexation Buffer to 10% of the culture volume. In parallel, volumes of Transfection Booster and Transfection Reagent corresponding to 0.3% and 0.6% of the culture volume were mixed. The DNA/Viral-Plex and the Transfection Booster/Transfection Reagent components were mixed at room temperature for 10 minutes and then incubated with the Gibco™ Viral Production Cells at room temperature for 20 minutes, after which the cells were returned to the humidified incubator. After 72 hours, AAV-Max Lysis Buffer was added at a volume corresponding to 10% of the culture volume to lyse cells. The cell lysate was subsequently added with 2 mM MgCl₂ and 90 U/mL benzonase endonuclease (GENIUS™ Nuclease) and incubated at 37° C. for 2 hours. The lysate was finally clarified via centrifugation at 4100 g for 40 minutes and the resulting supernatant was collected.

TABLE 1
Plasmid ratios for the production of different AAV serotypes.

| | | Plasmids Ratio |
|---|---|---|
| | | (pRC:pHelper:pAAV-GFP or |
| Serotype | Transfection | pRC + Helper:pAAV-GFP) |
| AAV1 | Triple | 2:0.5:1 |
| AAV2 | Triple | 2:0.5:1 |
| AAV3 | Double | 1:1 |
| AAV5 | Triple | 2:0.5:1 |
| AAV6 | Double | 1:1 |
| AAV7 | Triple | 2:0.5:1 |
| AAV8 | Triple | 2:0.5:1 |
| AAV9 | Triple | 2:0.5:1 |
| AAVrh.10 | Triple | 2:0.5:1 |

Preparation of feed samples. Pure AAV1, AAV2, AAV3, AAV5, AAV6, AAV7 AAV8, AAV9, and AAVrh.10 at the titer of ~5·10¹² vp/mL in 20 mM NaCl in 10 mM Bis-Tris buffer at pH 7.0, and AAV1, AAV5, AAV7, and AAV8 at the titer of ~5·10¹² vp/mL in 50 mM Acetate and 2 mM MgCl₂ at pH 5.0 were isolated from the HEK293 cell lysates prepared as described herein. Furthermore, the cell culture lysates of AAV2, AAV3, AAV6, AAV9, and AAVrh.10 were diafiltered against 20 mM NaCl in 10 mM Bis-Tris buffer at pH 7.0 to achieve a final AAV titer of ~5·10¹² vp/mL and an HCP titer of ~0.3 mg/mL.

Binding studies of AAV serotypes 1, 2, 3, 5, 6, 7, 8, 9, and rh.10 in non-competitive conditions using peptide-Toyopearl resins. A volume of 0.5 mL of Poros™ CaptureSelect™ AAVX affinity resin, AVB Sepharose HP resin, A10-Toyopearl, A11-Toyopearl, A12-Toyopearl, and A13-Toyopearl resins was flow-packed in an Alltech chromatography column and washed with 10 column volumes (CVs) of 20% v/v ethanol, 10 CVs of MilliQ water, and 10 CVs of binding buffer (20 mM NaCl in 10 mM Bis-Tris buffer at pH 7.0 or 2 mM MgCl₂ in 50 mM acetate buffer at pH 5.0). A volume of 10 mL of pure AAV solution in binding buffer prepared as described herein was loaded on the column at the flow rate of 0.17 mL/min (residence time, RT: 3 min). The resin was washed with 20 CVs of binding buffer at 0.5 mL/min. The bound AAVs were eluted from the peptide-Toyopearl resins using 0.4 M MgCl₂ in 10 mM Bis-Tris HCl buffer at pH 6.5 at the flow rate of 0.5 mL/min (RT: 1 min); and from Poros™ CaptureSelect™ AAVX affinity resin and AVB Sepharose HP resin respectively using 0.2 M MgCl₂ in 200 mM citrate buffer at pH 2.2 and PBS at pH 2.0 at the flow rate of 0.25 mL/min (RT: 2 min). All resins were regenerated with 10 CVs of phosphate-buffered saline at pH 2.0 at the flow rate of 0.5 mL/min. All dynamic binding experiments were performed using a ÄKTA Avant system from Cytiva (Marlborough, MA, USA) while continuously monitoring the effluent via UV spectrophotometry at 280 nm. The collected fractions were analyzed using a serotype specific AAV ELISA Kit as described herein to quantify the values of AAV binding and recovery.

Purification of AAV from clarified HEK293 using peptide-Toyopearl resins. A volume of 0.5 mL of Poros™ CaptureSelect™ AAVX affinity resin, AVB Sepharose HP resin, A1-Toyopearl, A3-Toyopearl, A4-Toyopearl, A6-Toyopearl, A10-Toyopearl, A11-Toyopearl, A12-Toyopearl, and A13-Toyopearl resins were flow-packed and equilibrated with binding buffer. Specifically, 20 mM NaCl in 10 mM Bis-Tris buffer at pH 7.0 was utilized as binding buffer for the purification of AAV2, AAV3, AAV6, AAV9, and AAVrh.10; whereas 2 mM MgCl₂ in 50 mM acetate buffer at pH 5.0 was utilized as binding buffer for the purification of AAV1, AAV5, AAV7, and AAV8. A volume of 10 mL of clarified HEK293 cell lysate (AAV titer of ~5·10¹² vp/mL; HCP titer of ~0.3 mg/mL) diafiltered against the appropriate binding buffer was loaded at the flow rate of 0.17 mL/min (RT: 3 min). After resin wash in binding buffer, the bound AAV2, AAV3, AAV6, AAV9, and AAVrh.10 were eluted 0.4 M MgCl₂ in 10 mM Bis-Tris HCl buffer at pH 6.5; whereas the bound AAV1, AAV5, AAV7, and AAV8 were released in a first elution step using 20 mM NaCl in 10 mM Bis-Tris buffer at pH 7.0 followed by a second elution step using 0.4 M MgCl₂ in 10 mM Bis-Tris HCl buffer at pH 6.5. After resin wash in binding buffer, the bound AAV2, AAV3, AAV6, AAV9, and AAVrh.10 were eluted 0.4 M MgCl₂ in 10 mM Bis-Tris HCl buffer at pH 6.5; whereas the bound AAV1, AAV5, AAV7, and AAV8 were released in a first elution step using 20 mM NaCl in 10 mM Bis-Tris buffer at pH 7.0 followed by a second elution step using 0.4 M MgCl₂ in 10 mM Bis-Tris HCl buffer at pH 6.5. AAV elution from Poros™ CaptureSelect™ AAVX affinity resin, AVB Sepharose HP resin was conducted using 0.2 M MgCl₂ in 200 mM citrate buffer at pH 2.2 and PBS at pH 2.0 at the flow rate of 0.25 mL/min (RT: 2 min), respectively. The column effluents were continuously monitoring the conductivity, pH, and UV absorbance of the column effluents at 260 and 280 nm. All resins were finally regenerated with 10 CVs of PBS buffer at pH 2.0 at the flow rate of 0.5 mL/min. The collected flow-through and elution fractions were analyzed by ELISA Kit and qPCR/ddPCR to determine the values of AAV yield, HEK293 ELISA Kit to quantify the values of HCP removal, size exclusion chromatography and steric exclusion chromatography to measure global product purity, and fluorescence flow cytometry to quantify the transduction efficiency of the eluted AAVs.

Alkaline stability of A10-Toyopearl resin. A volume of 1 mL of CVIDGSASTDDDRIC (A10′; SEQ ID NO: 33), CVIDGSSSTDDDRIC (A10″; SEQ ID NO: 34), and CVIDGSASTDDDHIC (A10′″; SEQ ID NO: 35) Toyopearl resins were flow-packed and equilibrated with binding buffer (i.e., 20 mM NaCl in 10 mM Bis-Tris buffer at pH 7.0 for the purification of AAV2 and AAV9; 2 mM MgCl₂ in 50 mM acetate buffer at pH 5.0 for the purification of AAV5 and AAV8). The chromatographic steps of loading, washing, and AAV elution were conducted as described herein. Cleaning-in-Place (CIP) was then conducted by flowing 15 CVs of 0.1 M NaOH (aq) at 1 mL/min followed by static incubation for 15 min. The resin was finally washed with 5 CVs of binding buffer. The collected flow-through and elution fractions were analyzed by ELISA Kit to determine the values of AAV yield, HEK293 ELISA Kit to quantify the values of HCP removal, and size exclusion chromatography to measure global product purity, and fluorescence flow cytometry to quantify the transduction efficiency of the eluted AAVs.

Quantification of AAV capsids (vp) via serotype-specific AAV ELISA kits. The flow-through, wash, and elution samples collected as described herein were analyzed using serotype-specific ELISA kits (PROGEN, Wayne, PA) following the manufacturer's protocol.

Quantification of AAV genomes (vg) via qPCR and ddPCR. The flow-through, wash, and elution samples collected as described herein were initially treated with TurboDNAse at 37° C. for 60 minutes. After the DNAse inactivation step, the samples underwent a Proteinase K treatment at 60° C. for 60 minutes. After the Proteinase K inactivation step, the samples were combined with EXPRESS qPCR SuperMix with Premixed ROX, custom TaqMan probe, and the primers listed in Table 2 and Table 3, and analyzed using a QuantStudio™ 7 Flex Real-Time PCR System (ThermoFisher Scientific). Plasmid pAAV-GFP was used as a standard.

TABLE 2
Primers and probe sequences for AAV viral genome
quantification via qPCR (from Addgene).

| Primer | DNA Sequence |
|---|---|
| Forward Primer | 5′-AGC AAA GAC CCC AAC GAG AA-3′ |
| | (SEQ ID NO: 36) |
| Reverse Primer | 5′-GGC GGC GGT CAC GAA-3′ |
| | (SEQ ID NO: 37) |
| Probe | 5′-CGC GAT CAC ATG GTC CTG CTG G-3′ |
| | (SEQ ID NO: 38) |

TABLE 3
Primers and probe sequences for AAV viral genome
quantification via qPCR (from Aldevron).

| Primer | DNA Sequence |
|---|---|
| Forward Primer | 5′-TCTGGGACACAAATTGGAATACAACT-3′ |
| | (SEQ ID NO: 39) |
| Reverse Primer | 5′-CGGGTCTTGAAGTTCACTTTGATTC-3′ |
| | (SEQ ID NO: 40) |
| Probe | 5′-CATGGCAGACAAACAA-3′ |
| | (SEQ ID NO: 41) |

The samples were also plated in triplicate with supermix, custom TaqMan probe, and the primers listed in Table 4. After sample loading, the plate was sealed, centrifuged, and placed in a QX600 AutoDG droplet generator (Bio Rad, Hercules, CA). After droplet generation, the plate was sealed and placed on the C1000 Thermal Cycler (Bio Rad). Finally, the plate was placed in the QX200 plate reader (Bio Rad) for data collection.

TABLE 4
Primers and probe sequences for AAV viral genome
quantification.

| Primer | DNA Sequence |
|---|---|
| Forward Primer | 5′-AGT AAC GCC AAT AGG GAC TTT C-3′ |
| | (SEQ ID NO: 42) |
| Reverse Primer | 5″ GGC GTA CTT GGC ATA TGA TAC A-3′ |
| | (SEQ ID NO: 43) |
| Probe | 5′-/56-FAM/TTA CGG TAA/ZEN/ACT GCC |
| | CAC TTG GCA/3IABkFQ/-3″ |
| | (SEQ ID NO: 44) |

Quantification of HEK293 HCPs. The feed, flow-through, wash, and elution samples collected as described herein was analyzed using a Generation 3 HEK293 HCP ELISA kit (Cygnus Technologies, Southport, NC) following the manufacturer's protocols.

Analytical size-exclusion chromatography (SEC-HPLC). The feed, flow-through, wash, and elution samples collected as described herein were analyzed by SEC HPLC using a BioResolve SEC column (Waters, Milford, MA) operated with a 40-min isocratic method using PBS at pH 7.0 (0.05% w/w sodium azide) at the flow rate of 0.50 mL/min. A volume of 10 μL of sample was injected and the effluent was continuously monitored via UV (260 nm and 280 nm) fluorescence spectroscopy (ex/em: 280/350 nm).

Analytical steric-exclusion chromatography (SXC). The feed, flow-through, and elution samples collected as described herein were analyzed via analytical SXC using a monolith 0.1 mL CIMac™ OH analytical column (BIA Separations, Slovenia) operated with a 7-min linear gradient from 100:0 A:B to 0:100 A:B (mobile phase A: 10% v/v PEG 6K in PBS at pH 7.0; mobile phase B: 3×PBS, 10% Isopropyl alcohol at pH 7.0) at the flow rate of 2.0 mL/min. Injection volumes were normalized based on the AAV capsid titer measured via ELISA kits as described herein. The effluent was continuously monitored via fluorescence spectroscopy (ex/em: 280/350 nm).

Fluorescence flow cytometry (FFC). HT1080 cells were cultured in DMEM media supplemented with 10% v/v FBS at 5% CO₂ and 37° C. until reaching 80-90% confluence. Cells were then seeded in 96-well plates at a density of 6,000 cells/well and cultured overnight. The AAVs in the HEK293 cell lysate and the eluted samples were serially diluted in DMEM (no FBS and antibiotics) and added with polybrene at 8 μg/mL. A volume of 0.1 mL of diluted AAV sample was incubated with the HT1080 cells. After 24 hrs, spent medium was replaced with fresh DMEM supplemented with 10% v/v FBS and the cells were cultured for 72 hrs. The fraction of cells expressing GFP (GFP⁺) was quantified using a CytoFlex flow cytometer (Beckman Coulter, Brea, CA) and the number of transduction units per mL (TU/mL) was calculated using Equation 2:

$$Activity ( \frac{TU}{mL} ) = \frac{{N}_{HT 1080} \times \% {GFP}^{+}}{V \times DF} \qquad Equation 2$$

Wherein N_{HT1080} is the number of cells incubated with the diluted AAV sample, V is the volume of the diluted AAV sample, and DF is the dilution factor.

In silico design of alkaline-stable variants of peptide A10. The crystal structures of AAVR in complex with AAV2 (3JIS, 6IHB and 6NZ0), AAV5 (7KP3 and 7KPN), and AAV9 (7WJX and 7WQP) as well as the complex of AAV2 with monoclonal antibody A20 (3JIS) were initially prepared using Protein Prep Wizard (PPW, Schrödinger, New York, NY). The resulting VPs were utilized to construct triangular clusters: AAV2 and AAV9 were conditioned to pH 7.4 whereas AAV5 and AAV8 were conditioned to pH 5.0 using PROPKA. Peptide CVIDGSQSTDDDKIC-GSG (A10; SEQ ID NO: 10) and its variants CVIDGSASTDDDRIC-GSG (A10′; SEQ ID NO: 33), CVIDGSSSTDDDRIC-GSG (A10″; SEQ ID NO: 34), and CVIDGSASTDDDHIC-GSG (A10′″; SEQ ID NO: 35) were constructed in Avogadro in the disulfide cyclic format Cys-X₁X₂[ . . . ]Xₙ-Cys-GSG (note: the disulfide bond was formed between the two Cys residues) and their structures were prepared in GROMACS using the force field GROMOS 54A7 by implementing the molecular modeling workflow. The structures with absolute energy minima were docked against the triangular clusters using the docking software HADDOCK (High Ambiguity Driven Protein-Protein Docking) v.2.4. The AAVR-binding residues and the A20-binding residues on the VP proteins and residues X₁X₂[ . . . ]Xₙ on the peptides were marked as “active”, while all other residues were marked as “passive”. The top AAV: peptide clusters were ranked using the dMM-PBSA score. The selected AAV: peptide complexes were finally refined via 200-ns MD simulations in explicit solvent conditions to estimate the free energy of binding (AGB).

### 4. Examples

Adeno-associated viruses (AAVs) have acquired a central role in modern medicine as delivery agents for gene therapies targeting rare diseases. While new AAVs are being introduced with improved tissue targeting, potency, and safety, the current technology for AAV manufacturing is modeled after conventional antibody bioprocessing: in particular, the purification pipeline hinges on protein ligands with high binding strength for the affinity-based capture step. While providing high AAV-binding capacity and selectivity, these ligands require strong acid (pH<3) elution conditions, thus compromising product activity and stability, and their low biochemical stability limits their lifetime in spite of their high cost. Seeking to introduce a more robust and affordable—yet equally effective—affinity technology, embodiments of the present disclosure provide a cohort of peptide ligands that (i) mimic the biorecognition activity of the AAV receptor (AAVR) and anti-AAV antibody A20, while (ii) enabling product elution under near-physiological conditions (pH 6.0) and (iii) granting extended reusability by withstanding multiple regenerations. A20-mimetic CYIHFSGYTNYNPSLKSC (SEQ ID NO: 1) and AAVR-mimetic CVIDGSQSTDDDKIC (SEQ ID NO: 10) demonstrated excellent capture of serotypes belonging to distinct clones/clades—AAV1, AAV2, AAV5, AAV6, AAV8, and AAV9—corroborating the in silico models documenting their ability to target regions of the virion proteins that are conserved across all serotypes. CVIDGSQSTDDDKIC-Toyopearl resin (SEQ ID NO: 10) features values of binding capacity (~10¹⁴ vp per mL) and product yields (~60-80%) on par with commercial adsorbents, and purified AAV2 from HEK293 and Sf9 cell lysates affording high recovery (up to 78%) and reduction of host cell proteins (up to 700-fold), and high transduction activity (up to 65%) of the purified viruses.

The accompanying Examples are offered as illustrative as a partial scope and particular embodiments of the disclosure and are not meant to be limiting of the scope of the disclosure.

### Example 1

Rational design of AAVR-mimetic and A20-mimetic peptides. A number of biological ligands targeting AAV are known to date, including transmembrane receptor proteins and engineered proteins. Tissue targeting and cell access by AAVs is mediated by attachment factors, namely glycan moieties (e.g., sucrose octasulfate, sialic acid, and galactose), also known as ‘primary receptors’, which feature promiscuous low-affinity capsid binding and whose role is to accumulate AAV at the cell surface; and cell surface receptors that specifically interact with AAV and whose binding is required to initiate viral cell entry. To date, known receptors and their target serotypes include oligosaccharide heparin (AAV2) and fondaparinux (AAV-DJ) as well as the AAV receptor (AAVR, also known as KIAA0319L), a 150 kDa glycoprotein required for cell transduction by several serotypes, including AAV1, AAV2, AAV3B, AAV5, AAV6, AAV8, and AAV9. The ectodomain of AAVR comprises multiple domains, known as Ig-like polycystic kidney disease repeat domains (PKD): for example, AAV2 is bound by PDK1-3, AAV5 predominantly interacts with PKD1, while AAV1 and AAV8 require a combination of PKD1 and PKD2. Additionally, AAV2 targets integrins, fibroblast growth factor receptor (FGFR1), αVβ5, and α5β1; AAV2, AAV3, AAV8, and AAV9 capsid proteins have been found to bind the laminin receptor displayed on the surface of yeast cells; AAV2 and AAV3 bind FGFR1, the hepatocyte growth factor receptor (HGFR), while AAV5 and AAV6 respectively target the platelet-derived growth factor receptor (PDGFR) and the epidermal growth factor receptor (EGFR). Besides natural receptors, a number of anti-AAV monoclonal neutralizing antibodies have been developed, including mNAbs ADKla, 4E4, and ADK6 targeting AAV1, mNAbs A20 and C37-B targeting AAV2, mNAbs ADK5b and HL2476 and mAbs ADK5a and 3C5 targeting AAV5, and mNAbs ADK5a and ADK6 targeting AAV6, and mAb ADK8 targeting AAV8. Finally, single chain camelid antibody fragments and small protein scaffolds have been developed as affinity ligands for purifying AAVs from recombinant cell lysates, including serotype-agnostic AAVX, AAV8- and AAV9-targeted CSAL8 and CSAL9 developed by ThermoFisher; serotype-agnostic AVB by Cytiva; and AVIPure® AAV2, AAV8, and AAV9 for the corresponding serotypes.

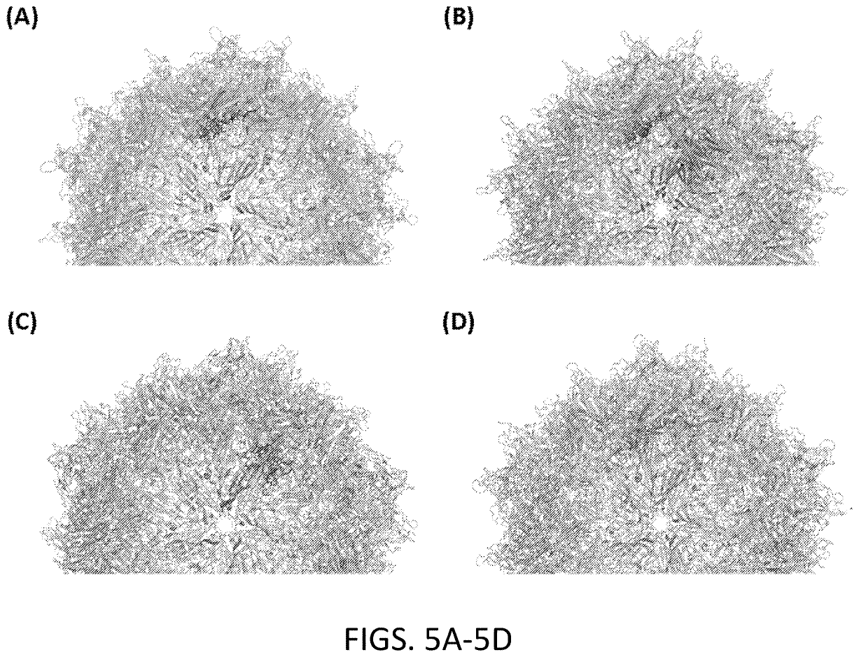

Despite the abundance of AAV-binding ligands, only the crystal structures of AAVR in complex with AAV1 (PDB ID: 6JCQ and 7T15), AAV2 (6IHB and 6NZ0), AAV5 (6JCS), and AAV9 (7WJX) as well as the complex of AAV2 with monoclonal antibody A20 (3J1S) are reported. The analysis of pairwise interactions between the active residues on AAVR and A20 and the targeted residues on the virion protein (VP1), reported in FIG. 5 and Table 8, identified critical AAV-binding residues and motifs. These were utilized to design an in silico ensemble of candidate ligands, whose sequence, structure, and key physicochemical parameters are reported in Table 9. As can be gathered from the sequence homology and the values of root-mean-square deviation (RMSD) of the atomic positions of the peptides vs. their cognate proteins, the proposed library spans a wide space of chemical and structural diversity as well as different levels of similarity with A20 and AAVR.

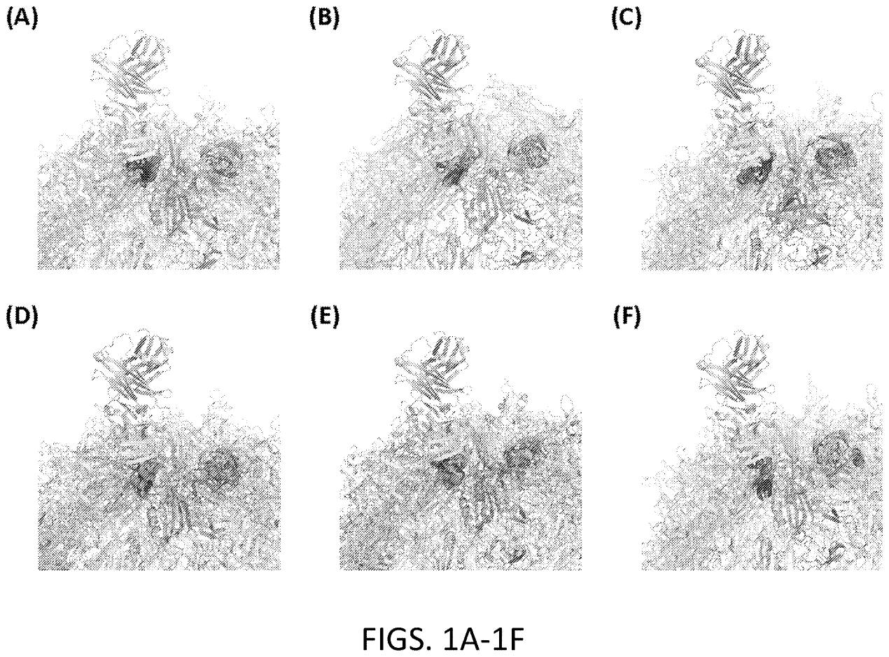

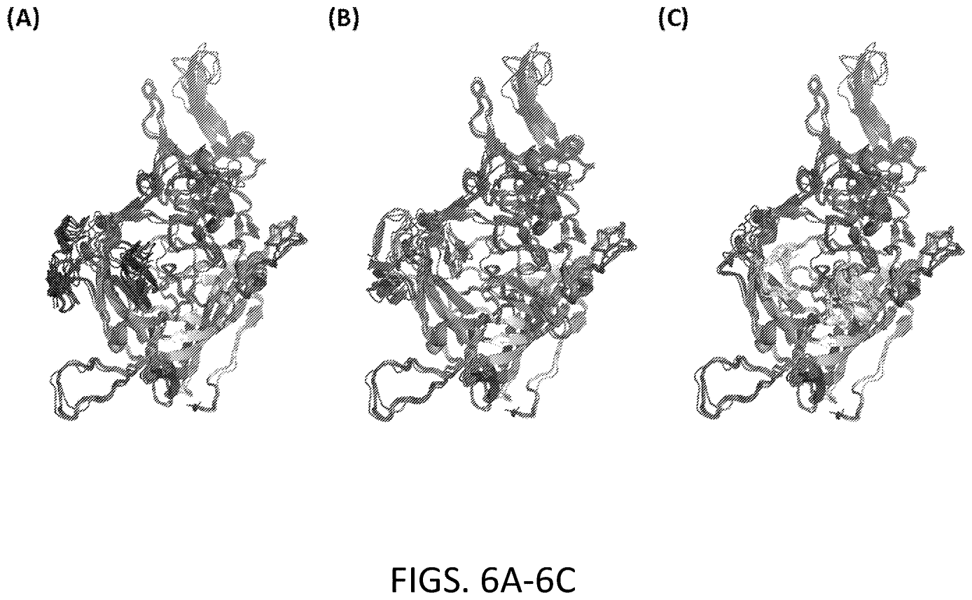

The peptides were docked in silico against the homology spherical cap structures of AAV1, AAV2, AAV5, AAV6, AAV8, and AAV9. These were created by collating the published structures of VP1 into the triangular asymmetric units that form the icosahedral AAV capsid. Prior to docking, the triangular clusters were equilibrated to two values of pH—7.4, which is utilized during adsorption, and 6.0, which is adopted for the product release. An initial round of “blind” docking was performed to evaluate the ability of the designed sequences to target the known binding sites of A20 and AAVR. In order to mimic the orientational constraint imposed upon the peptides by their conjugation onto the surface of the chromatographic resin, the -GSG tripeptide appended on the C-terminal end of the peptides was constrained not to bind AAV. Selected AAV:peptide complexes (those comprising more than 20 peptide clusters) were refined via 250-ns MD simulations in explicit solvent at both pH 7.4 and 6.0 to obtain reliable values of binding free energy (ΔG_{b}). Representative complexes formed by the selected peptides on the target serotypes are shown in FIG. 1, while the values of binding energy and the corresponding dissociation constant (K_{D,in silico}) are listed in Table 5; finally, detailed results of A1, A4, and A10 docking on the target AAV1, AAV2, AAV5, AAV6, AAV8, and AAV9 are reported in FIG. 6.

TABLE 5
Values of dissociation constant (K_{D,in silico}) of the complexes formed by A20-mimetic peptides with the
capsids of AAV1, AAV2, AAV5, AAV6, AAV8, and AAV9 obtained via molecular docking and dynamics
simulations at pH 7.4 and ionic strength of 150 mM and at pH 6.0 and ionic strength of 2M.

| | AAV1:peptide | | AAV2:peptide | | AAV5:peptide | |
|---|---|---|---|---|---|---|
| A20-mimetic | K_{D,in silico} (M) | | K_{D,in silico} (M) | | K_{D,in silico} (M) | |
| Sequence | pH 6.0 | PH 7.4 | PH 6.0 | PH 7.4 | pH 6.0 | PH 7.4 |
| YIHFSGYTNYNPSLKS | 8.81 · 10⁻⁴ | 2.16 · 10⁻⁶ | 7.91 · 10⁻⁴ | 6.12 · 10⁻⁷ | 3.83 · 10⁻³ | 1.84 · 10⁻⁶ |
| (A1) | | | | | | |
| (SEQ ID NO: 1) | | | | | | |
| YIHFSGYTNYNGSLKS | 4.88 · 10⁻⁴ | 1.10 · 10⁻⁶ | 4.69 · 10⁻⁴ | 1.07 · 10⁻⁶ | 4.59 · 10⁻⁴ | 1.08 · 10⁻⁶ |
| (A2) | | | | | | |
| (SEQ ID NO: 2) | | | | | | |
| YVHFSGYSNYSPS (A3) | 1.75 · 10⁻³ | 9.65 · 10⁻⁵ | 4.27 · 10⁻⁴ | 7.47 · 10⁻⁷ | 5.67 · 10⁻⁴ | 8.58 · 10⁻⁶ |
| (SEQ ID NO: 3) | | | | | | |
| YGHFSGYGNYGP (A4) | 5.70 · 10⁻⁴ | 2.64 · 10⁻⁵ | 6.53 · 10⁻⁴ | 6.17 · 10⁻⁶ | 1.26 · 10⁻⁴ | 3.72 · 10⁻⁵ |
| (SEQ ID NO: 4) | | | | | | |
| YGHFSPYGNYGP (A5) | 7.21 · 10⁻⁴ | 4.18 · 10⁻⁵ | 7.27 · 10⁻⁴ | 4.21 · 10⁻⁵ | 7.77 · 10⁻⁴ | 4.20 · 10⁻⁵ |
| (SEQ ID NO: 5) | | | | | | |
| YIHFSGYTNYNP (A6) | 8.77 · 10⁻⁴ | 8.01 · 10⁻⁵ | 8.27 · 10⁻⁴ | 1.34 · 10⁻⁶ | 8.31 · 10⁻⁴ | 9.12 · 10⁻⁵ |
| (SEQ ID NO: 6) | | | | | | |
| YIHFSPYTNYNP (A7) | 1.09 · 10⁻⁴ | 6.31 · 10⁻⁵ | 5.21 · 10⁻⁵ | 2.45 · 10⁻⁵ | 9.24 · 10⁻⁵ | 5.87 · 10⁻⁵ |
| (SEQ ID NO: 7) | | | | | | |
| YHFSYNYPKS (A8) | 5.89 · 10⁻⁴ | 1.57 · 10⁻⁵ | 5.99 · 10⁻⁴ | 1.65 · 10⁻⁵ | 6.18 · 10⁻⁴ | 1.72 · 10⁻⁵ |
| (SEQ ID NO: 8) | | | | | | |
| YHFSYNYP (A9) | 6.79 · 10⁻⁴ | 2.84 · 10⁻⁵ | 6.61 · 10⁻⁴ | 2.87 · 10⁻⁵ | 6.85 · 10⁻⁴ | 2.94 · 10⁻⁵ |
| A20 | † | | | | | |
| | AAV6:peptide | | AAV8:peptide | | AAV9:peptide | |
| A20-mimetic | K_{D,in silico} (M) | | K_{D,in silico} (M) | | K_{D,in silico} (M) | |
| Sequence | pH 6.0 | PH 7.4 | pH 6.0 | PH 7.4 | pH 6.0 | PH 7.4 |
| YIHFSGYTNYNPSLKS | 5.28 · 10⁻⁴ | 2.54 · 10⁻⁷ | 4.95 · 10⁻⁴ | 6.26 · 10⁻⁶ | 1.27 · 10⁻⁴ | 6.27 · 10⁻⁶ |
| (A1) | | | | | | |
| (SEQ ID NO: 1) | | | | | | |
| YIHFSGYTNYNGSLKS | 1.04 · 10⁻⁴ | 1.10 · 10⁻⁶ | 4.59 · 10⁻⁴ | 1.03 · 10⁻⁶ | 4.56 · 10⁻⁴ | 1.09 · 10⁻⁶ |
| (A2) | | | | | | |
| (SEQ ID NO: 2) | | | | | | |
| YVHFSGYSNYSPS (A3) | 4.43 · 10⁻⁴ | 2.84 · 10⁻⁶ | 7.47 · 10⁻⁵ | 4.28 · 10⁻⁶ | 1.09 · 10⁻⁴ | 5.41 · 10⁻⁵ |
| (SEQ ID NO: 3) | | | | | | |
| YGHFSGYGNYGP (A4) | 5.26 · 10⁻⁴ | 1.38 · 10⁻⁶ | 4.50 · 10⁻⁴ | 3.44 · 10⁻⁶ | 2.64 · 10⁻⁴ | 4.39 · 10⁻⁶ |
| (SEQ ID NO: 4) | | | | | | |
| YGHFSPYGNYGP (A5) | 4.32 · 10⁻⁴ | 4.36 · 10⁻⁵ | 7.64 · 10⁻⁴ | 4.18 · 10⁻⁵ | 7.34 · 10⁻⁴ | 4.15 · 10⁻⁵ |
| (SEQ ID NO: 5) | | | | | | |
| YIHFSGYTNYNP (A6) | 4.74 · 10⁻⁴ | 2.20 · 10⁻⁶ | 4.06 · 10⁻⁴ | 3.47 · 10⁻⁵ | 1.21 · 10⁻⁴ | 4.56 · 10⁻⁵ |
| (SEQ ID NO: 6) | | | | | | |
| YIHFSPYTNYNP (A7) | 4.01 · 10⁻⁵ | 2.54 · 10⁻⁵ | 8.03 · 10⁻⁵ | 4.60 · 10⁻⁵ | 1.04 · 10⁻⁴ | 4.50 · 10⁻⁵ |
| (SEQ ID NO: 7) | | | | | | |
| YHFSYNYPKS (A8) | 1.66 · 10⁻⁴ | 1.59 · 10⁻⁵ | 5.79 · 10⁻⁴ | 1.67 · 10⁻⁵ | 6.15 · 10⁻⁴ | 1.59 · 10⁻⁵ |
| (SEQ ID NO: 8) | | | | | | |
| YHFSYNYP (A9) | 2.82 · 10⁻⁴ | 2.92 · 10⁻⁵ | 7.03 · 10⁻⁴ | 2.87 · 10⁻⁵ | 6.84 · 10⁻⁴ | 2.98 · 10⁻⁵ |

| | |
|---|---|
| A20 | † |

†: not available.

TABLE 6
Values of dissociation constant (K_{D,in silico}) of the complexes formed by AAVR-mimetic peptides with the
capsids of AAV1, AAV2, AAV5, AAV6, AAV8, and AAV9 obtained via molecular docking and dynamics
simulations at pH 7.4 and ionic strength of 150 mM and at pH 6.0 and ionic strength of 2M.

| | AAV1:peptide | | AAV2:peptide | | AAV5:peptide | |
|---|---|---|---|---|---|---|
| AAVR−mimetic | KD,in silico (M) | | KD,in silico (M) | | KD,in silico (M) | |
| Sequence | pH 6.0 | PH 7.4 | pH 6.0 | PH 7.4 | pH 6.0 | PH 7.4 |
| VIDGSQSTDDDKI (A10) | 3.84 · 10⁻⁴ | 1.18 · 10⁻⁵ | 4.66 · 10⁻⁴ | 4.15 · 10⁻⁶ | 6.11 · 10⁻⁴ | 9.83 · 10⁻⁶ |
| (SEQ ID NO: 10) | | | | | | |
| DGSQSTDDDKI (A11) | 3.75 · 10⁻⁴ | 1.10 · 10⁻⁵ | 5.31 · 10⁻⁴ | 2.92 · 10⁻⁶ | 6.98 · 10⁻⁴ | 9.90 · 10⁻⁵ |
| (SEQ ID NO: 11) | | | | | | |
| DSQSTDDDKI (A12) | 1.58 · 10⁻⁴ | 1.29 · 10⁻⁴ | 5.43 · 10⁻⁴ | 1.40 · 10⁻⁶ | 5.71 · 10⁻⁴ | 9.62 · 10⁻⁵ |
| (SEQ ID NO: 12) | | | | | | |
| SGSTDDDKI (A13) | 9.45 · 10⁻⁴ | 1.03 · 10⁻⁴ | 5.80 · 10⁻⁴ | 1.59 · 10⁻⁶ | 3.23 · 10⁻⁴ | 1.24 · 10⁻⁵ |
| (SEQ ID NO: 13) | | | | | | |
| GYWIGPFTGGGYIHFSGYT | 3.52 · 10⁻³ | 7.58 · 10⁻⁵ | 3.81 · 10⁻⁴ | 7.57 · 10⁻⁵ | 2.82 · 10⁻³ | 7.26 · 10⁻⁵ |
| (A14) | | | | | | |
| (SEQ ID NO: 14) | | | | | | |
| GYWIGPFTGPGYIHFSGYT | 6.80 · 10⁻³ | 4.81 · 10⁻⁵ | 6.37 · 10⁻³ | 4.95 · 10⁻⁵ | 3.69 · 10⁻³ | 4.98 · 10⁻⁵ |
| (A15) | | | | | | |
| (SEQ ID NO: 15) | | | | | | |
| GYWIGPFTPGPYIHFSGYT | 3.08 · 10⁻³ | 6.30 · 10⁻⁵ | 3.23 · 10⁻³ | 6.71 · 10⁻⁵ | 2.14 · 10⁻³ | 6.61 · 10⁻⁵ |
| (A16) | | | | | | |
| (SEQ ID NO: 16) | | | | | | |
| LITHPRDYSPKLTPGLYEFG | 4.95 · 10⁻³ | 2.71 · 10⁻⁵ | 4.70 · 10⁻³ | 2.75 · 10⁻⁵ | 1.01 · 10⁻³ | 2.74 · 10⁻⁵ |
| (A17) | | | | | | |
| (SEQ ID NO: 17) | | | | | | |
| AAVR | 1.40 · 10⁻⁵ | 1.80 · 10⁻⁶ | 1.17 · 10⁻⁵ | 1.60 · 10⁻⁶ | 9.01 · 10⁻⁶ | 1.71 · 10⁻⁶ |
| | AAV6:peptide | | AAV8:peptide | | AAV9:peptide | |
| AAVR−mimetic | KD,in silico (M) | | KD,in silico (M) | | KD,in silico (M) | |
| Sequence | pH 6.0 | PH 7.4 | pH 6.0 | PH 7.4 | pH 6.0 | pH 7.4 |
| VIDGSQSTDDDKI (A10) | 5.41 · 10⁻⁴ | 1.29 · 10⁻⁵ | 2.21 · 10⁻⁴ | 8.06 · 10⁻⁶ | 6.78 · 10⁻⁴ | 3.08 · 10⁻⁶ |
| (SEQ ID NO: 10) | | | | | | |
| DGSQSTDDDKI (A11) | 4.10 · 10⁻⁴ | 1.93 · 10⁻⁶ | 3.28 · 10⁻⁴ | 7.29 · 10⁻⁵ | 3.16 · 10⁻⁴ | 7.97 · 10⁻⁶ |
| (SEQ ID NO: 11) | | | | | | |
| DSQSTDDDKI (A12) | 3.45 · 10⁻⁴ | 1.56 · 10⁻⁶ | 2.37 · 10⁻⁴ | 7.28 · 10⁻⁵ | 3.52 · 10⁻⁴ | 8.01 · 10⁻⁶ |
| (SEQ ID NO: 12) | | | | | | |
| SGSTDDDKI (A13) | 3.59 · 10⁻⁴ | 1.63 · 10⁻⁶ | 2.06 · 10⁻⁴ | 7.16 · 10⁻⁵ | 4.40 · 10⁻⁴ | 8.56 · 10⁻⁶ |
| (SEQ ID NO: 13) | | | | | | |
| GYWIGPFTGGGYIHFSGYT | 2.80 · 10⁻³ | 7.82 · 10⁻⁵ | 2.80 · 10⁻³ | 7.82 · 10⁻⁵ | 2.66 · 10⁻³ | 7.78 · 10⁻⁵ |
| (A14) | | | | | | |
| (SEQ ID NO: 14) | | | | | | |
| GYWIGPFTGPGYIHFSGYT | 3.82 · 10⁻³ | 4.73 · 10⁻⁵ | 3.84 · 10⁻³ | 4.98 · 10⁻⁵ | 3.76 · 10⁻³ | 4.74 · 10⁻⁵ |
| (A15) | | | | | | |
| (SEQ ID NO: 15) | | | | | | |
| GYWIGPFTPGPYIHFSGYT | 2.09 · 10⁻³ | 6.73 · 10⁻⁵ | 2.22 · 10⁻³ | 6.63 · 10⁻⁵ | 2.15 · 10⁻³ | 6.38 · 10⁻⁵ |
| (A16) | | | | | | |
| (SEQ ID NO: 16) | | | | | | |
| LITHPRDYSPKLTPGLYEFG | 9.98 · 10⁻³ | 2.77 · 10⁻⁵ | 1.02 · 10⁻³ | 2.70 · 10⁻⁵ | 1.02 · 10⁻³ | 2.72 · 10⁻⁵ |
| (A17) | | | | | | |
| (SEQ ID NO: 17) | | | | | | |

| | | | |
|---|---|---|---|
| AAVR | † | 3.57 · 10⁻⁶ | 1.70 · 10⁻⁵ |

†: not available.

As shown by Tables 5 and 6, a significant fraction of the designed peptides—17 sequences out of the 28 originally designed—were found to bind all target serotypes, forming binding poses that overlap with those of the AAVR: AAV and A20: AAV2 complexes (FIG. 1). Most notably, nine A20-mimetic peptides formed true affinity interactions (|ΔG_{b}|>6.5 kcal/mol at pH 7.4) with all serotypes, despite the cognate A20 being designed as an anti-AAV2 antibody: this can be attributed to the cyclic format of these candidate ligands, whose rigidity decreases the entropic penalty to the binding energy that is characteristic of their linear, and hence more flexible, counterparts; however, shorter sequences (A5-A9) exhibited lower binding energies (binding energy |ΔG_{b}|<7.5 kcal/mol) than longer variants (A1-A4), whose higher number of amino acids provides a stronger enthalpic contribution to the binding energy. Particularly notable was the case of CYIHFSGYTNYNPSLKSC (A1; SEQ ID NO: 1), whose dominant binding site on AAV1, AAV2, AAV5, AAV6, and AAV8 (|ΔG_{b}|~7.7-9 kcal/mol at pH 7.4) shares >90% of paired interactions with A20. It was also noted that the shortest A20-mimetics (A6-A9) showed the ability to form multiple low-affinity binding poses on the various serotypes that do not overlap with the binding sites of either A20 or AAVR. This can be imputed to their smaller hydrodynamic radius, which enables these peptides to fit druggable sites displayed on the capsid surface that are precluded to larger binders. The solvent-accessible convex surface of AAV capsids present multiple sites that are highly conserved across serotypes and “ligandable” (i.e., and whose physicochemical features—namely, pocket surface and volume as well as balance of electrostatic, hydrophobic and hydrogen bond-forming residues—are suitable to accommodate peptide ligands); since the target regions of AAVR and A20 are included in the list of ligandable sites, it did not surprise that A20-mimetic peptides interact with multiple sites on all serotypes. At the same time, this also suggests the possibility for smaller A20-mimetic peptides to interact with host cell proteins (HCPs) and other impurities in the feedstock, thus reducing their binding selectivity and thwarts their candidacy for experimental evaluation.

Analogous results were observed with the AAVR-mimetic sequences. Among them, CVIDGSQSTDDDKIC (A10; SEQ ID NO: 10) and its derivatives A11-A13 featured affinity-like interactions (|ΔG_{b}|~6.5-7.7 kcal/mol at pH 7.4) with all target serotypes, especially AAV2, AAV5, AAV6, AAV8, and AAV9, with which they formed binding poses that share >90% of paired interactions with AAVR. Once again, shorter variants, chiefly A12 and A13 were found to form multiple yet low-affinity interactions with multiple sites, besides the epitopes of A20 and AAVR, suggesting their ability to act as promiscuous binders, and thus as ligands with poor selectivity.

While the predicted affinity of these sequences was found to be consistently lower than that of A20-mimetics, it was noted that the binding strength of the AAV:AAVR complexes whence these peptides were derived is originally lower than that of the AAV2:A20 complex. Furthermore, a milder binding strength does not necessarily translate into weaker binding; the peptide density on the surface of the resin is sufficient to form multiple interactions with a single capsid, wherein multiple affinity interactions with modest binding energy are synergized into a strong avidity-like binding that efficient AAV capture (note: the values of peptide density on the resin (~0.12-0.15 mmol per gram), the resin's specific surface (~30 m²/g), and the projection area of the triangular unit formed by 3 VPs on the icosahedral capsid (~81 nm²), in fact, suggest that up to 30 peptides are displayed on pore surface that is impacted by a single capsid, enabling the formation of 3-5 VP:peptide interactions per bound capsid). Finally, moderate binding strength is welcome in the context of affinity purification of AAVs, since weak VP:peptide interactions reduce the risk of irreversible adsorption and promote an easier elution of the capsids, thus safeguarding their tissue tropism and transduction activity.

These observations motivate the selection of sequences whose binding energy decreases to ~4-5 kcal/mol as the pH decreases from pH 7.4 to 6.0 and the ionic strength of the aqueous environment increases to 2 M (representing the transition from the adsorption step conducted in PBS at pH 7.4 to the elution step in 1 M MgCl₂ at pH 6.0). This translates in a 120-to-330-fold shift in the dissociation constant for A20-mimetics (K_{D}, Table 5) and a 30-to-190-fold shift in the K_{D} of AAVR-mimetics (Table 6). A20-mimetics A1-A4 and AAVR-mimetics A10-A12 are the sequences with the strongest variation in binding energy (ΔΔG_{b}>3 kcal/mol, translating in a ΔK_{D}>150-fold increase), and thus the highest likelihood of releasing the bound capsids under the desired conditions. The mechanism of VP:peptide dissociation portrayed by the MD simulations is a combined results of the variation in pH and ionic strength. Contrary to what generally observed with peptide ligands, the Coulombic interactions formed by A20 and its mimetics A1-A4 provided a rather minor contribution (11-15%) to the binding energy at pH 7.4: the only interactions found were formed by cationic Lys in A1 and A2 with Asp514 and Asp 711 on AAV1, Asp269 and Asp 711 on AAV2, Asp704 on AAV5, Asp268 on AAV6, Asp270 on AAV8, and Asp231 on AAV9; conversely, A3 and A4 do not contain ionizable residues (except His, which is neutral at pH 7.4); finally, the triplet (DDD) of A10-A12 only targeted Lys508 on AAV1 and AAV2, Lys501 on AAV5, Lys507 on AAV6, and Lys509 on AAV8. Conversely, a strong network of hydrogen bonds and polar interactions formed by the side chains of Ser and Thr, Asn and Gln, His, Asp, and Tyr residues as well as the backbone amide bonds contribute ~65-74% of the binding energy. Finally, moderate hydrophobic interactions and π-π stacking occur, which account for ~16-24% of the binding energy. Finally, it was noted that the acidification of the environment to pH 6 causes a minor rearrangement in the capsid structure, together with softening most of the electrostatic interactions, whereas the addition of MgCl₂—a known chaotrope—destabilizes the electrostatic, hydrophobic, and hydrogen bonding interactions that maintain the native VP conformation and its interaction with the surface-bound peptide ligands, ultimately triggering the release of the capsid. These phenomena are observed mostly with the VP:A2, VP:A3, and VP:A10 complexes, whose binding strength at pH 7.4 is moderate (i.e., 7.6>|ΔG_{b}|>6.5 kcal/mol) and undergoes the sharpest change upon switching from binding to the elution conditions. For reference, the VP:A20 complex features an in silico ΔG_{b}~8.4 kcal/mol and drops by only 1.2 kcal/mol upon elution; the binding strength of AAV:AAVR complexes, while more modest, exhibits no-to-little dependence upon either pH or ionic strength within the explored ranges, and are therefore unlikely to afford sufficient product yield upon mild elution conditions.

In summary, these results support the adoption of small cyclic peptides as ligands for AAV purification via affinity chromatography: (i) with multiple μM-level affinity interactions cooperating into sub-nM avidity capture, peptide-functionalized adsorbents can match their protein-based counterparts in terms of binding capacity and selectivity; (ii) small pH variations or additions of chaotropes that disrupt the single VP:peptide interaction are sufficient to trigger capsid release; conversely, mild elution conditions only marginally affect VP:protein interactions, which require much stronger stimuli, resulting in a loss of yield and transduction activity of the recovered capsids as well as a shorter resin lifetime (note: irreversible multi-site VP:proteins interactions may in fact form a fouling film of bound capsids, which builds up across multiple uses and results in the short lifetime characteristic of commercial adsorbents).

### Example 2

Evaluation of AAV binding by the designed peptide ligands in non-competitive mode. The results of molecular docking and dynamics of designed peptides on multiple serotypes yielded a shortlist of sequences—namely, A20-mimetic peptides CYIHFSGYTNYNPSLKSC (A1; SEQ ID NO: 1), CYVHFSGYSNYSPSC (A3; SEQ ID NO: 3), CYGHFSGYGNYGPC (A4; SEQ ID NO: 4), and CYIHFSGYTNYNPC (A6; SEQ ID NO: 6), and AAVR-mimetic peptides CVIDGSQSTDDDKIC (A10; SEQ ID NO: 10) and CDSQSTDDDKIC (A12; SEQ ID NO: 12)—to be evaluated in dynamic mode against target serotypes AAV1, AAV2, AAV5, AAV6, AAV8 and AAV9. These targets belong to different clones (AAV5) or clades—namely A (AAV1 and AAV6), B (AAV2), E (AAV8), and F (AAV9)—and were selected for their outstanding therapeutic value: serotypes 1 and 2 target skeletal, muscle and cardiac cells, and are currently utilized in clinical trials against heart failure, Pompe disease, Hemophilia B, and AAT deficiency; serotypes 1, 2, 5, 8, 9 target the cells in the central nervous system, especially neurons, and are currently being tested in clinical trials against Alzheimer, Canavan, and Parkinson diseases; finally, serotypes 6 targets epithelial, skeletal cells, and hepatocytes, while serotype 8 targets cardiac cells, skeletal cells, hepatocytes. Notably, affinity resins marketed as serotype-agnostic show excellent binding of AAV1, AAV2, AAV5, and AAV6, but may struggle to capture AAV8 and AAV9, and dedicated adsorbents for their purification have been developed. Accordingly, the model AAVs adopted in the present disclosure, while only representing half of the wild serotypes, provide a broad coverage of the AAV atlas and thus adequate evaluation of the AAV-targeting activity of the designed sequences.

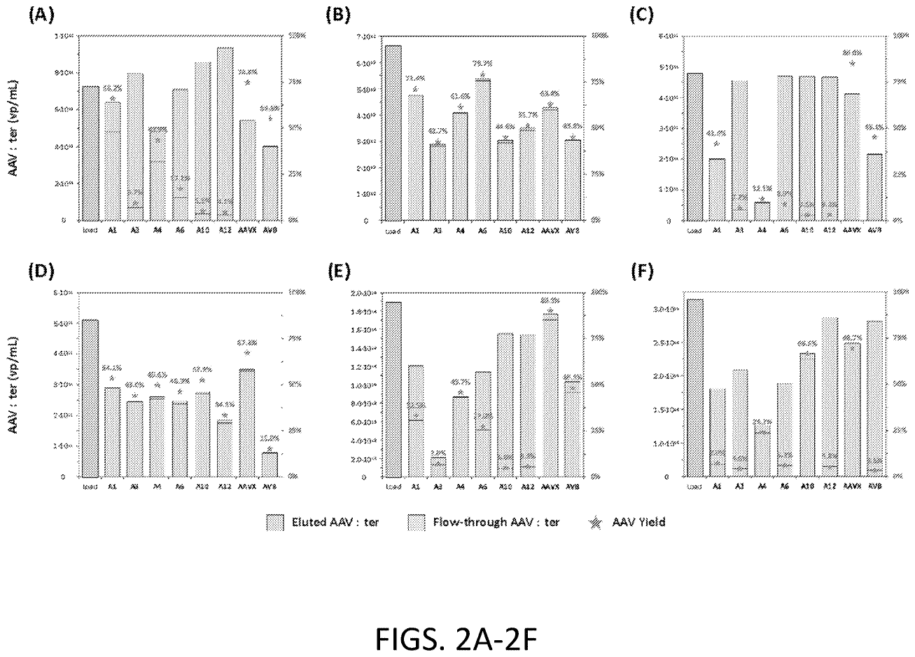

The peptides were conjugated on Toyopearl NH2-750F resin, whose large pore diameter (>100 nm) and small particle size ensures efficient AAV transport into and binding onto the adsorbent pores. To evaluate the peptide-based resins under conditions that are representative of biopharmaceutical processes, the feedstocks were formulated as pure AAVs at ~5.0·10¹¹-5.0·10¹² vp/mL in 10 mM Bis-Tris buffer at pH 7.0 and loaded at the ratio of ~10¹³ vp per mL of resin (the expected to be the average binding capacity of the resins). To ensure a stringent evaluation of the performance of peptide-based adsorbents, the bound AAVs were eluted from the peptide-Toyopearl resins under the same conditions adopted for peptide design—namely, 1 M MgCl₂ in 10 mM Bis-Tris buffer at pH 6.0—whereas a strong acidic buffer (i.e., 200 mM MgCl₂ in 200 mM citrate buffer at pH 2.2 and PBS at pH 2.0, respectively, as recommended by the manufacturers) was used for elution from the POROS™ CaptureSelect™ AAVX and AVB Sepharose HP resins used as reference adsorbents. The values of product loss (i.e., in the flow-through fractions) and yield are summarized in FIG. 2.

As shown in FIG. 2, the selected peptide resins, and particularly A1-Toyopearl, displayed broad AAV-binding activity. Particularly efficient is the binding of AAV2, which, followed by AAV6, was the serotype most efficiently captured. These results align with the rationale of ligand design, which leverages the known epitopes on AAVR and A20—both AAV2-binding proteins. Among the tested sequences, negligible values of product loss during loading and remarkable values of yield at pH 6.0 were provided by peptides A1 (0.14% and 71.4%, respectively), A4 (0.2% and 61.6%), and A6 (1.36%, 79.7%), which outperformed AAVX POROS™ (1.27% and 63.4%) and AVB Sepharose (0.2%, 45.8%) resins. Notably, the in silico results suggest that the list of binding sites of these peptides include, besides those targeted by AAVR and A20, two epitopes located on VP surface and distance from the VP: VP interface. The availability of neighboring binding sites promotes capsid capture, translating in higher values of capacity and lower loss during loading. At the same time, the significant drop in binding strength upon mild acidification is also coherent with the excellent values of product recovery. Supporting the efficient capture of AAV2 by almost all peptides is also the propensity of this serotype to aggregate into soluble multimeric constructs, which further promotes multi-site interactions with the peptide-functionalized surface.

Efficient binding and recovery were also observed with AAV1, AAV6, and AAV8. In particular, peptides A1 and A4 afforded excellent binding and gentle release of AAV1 and AAV6: the values of product yield were respectively 66.2 and 54.1% with A1-Toyopearl resin, which performed comparably to AAVX POROS™ resin (74.8 and 67.4%) and outperformed AVB Sepharose (55.5 and 15.0%); and 43.9% and 49.6% with A4-Toyopearl resin. Both serotypes belong to Clade A, which is closely related to Clade B, to which AAV2 belongs: AAV1 and AAV6 feature, in fact, a 91-92% structural homology with AAV2 (note: sequence homology, however, is 81-83%). With structural complementarity playing a key role in AAV docking on the peptide-functionalized surface, it stands to reason that structural homology is an underlying factor of AAV1/2/6 binding shared by these three sequences.

Peptides A1 and A4 also showed a similar performance with AAV8, affording product yield of 32.5% and 45.7% respectively, followed by A6 with 27%. Notably, AA8 (Clade E) exhibits a 94% structural homology, but only 82% sequence homology, with AAV2. When tested against AAV9, however, only A4 maintained acceptable product recognition with only 4.2% loss and 24.7% yield; conversely, A1 only afforded 7% yield and significant product loss. Notably, A10 returned a remarkable yield of 66.5%, comparable with that of AAVX POROS™ resin (69.7%) and significantly higher than that of AVB Sepharose resin (3.5%).

The capture of AAV5 proved the most challenging: the most unique among all AAVs, AAV5 exhibits a poor structural (58-71%) and sequence (58-79%) homology with the other serotypes evaluated in the present disclosure. It is therefore remarkable that peptide A1 captured (0.2% loss) and released it efficiently (41.6% yield).

Three general conclusions can be drawn from the experimental evaluation of the in silico-selected peptides. First, peptide CYGHFSGYGNYGPC (A4; SEQ ID NO: 4) performed well with all serotypes, exhibiting negligible product loss, thus suggesting that the variations in yield should be attributed to differences in binding strength to the various serotypes. A comparative evaluation of the values of K_{D} measured in silico (Table 5) and the experimental values of AAV yield, in fact, show that high yields (45-80%) were obtained when a >100-fold increase in K_{D} was registered (AAV2, AV6, and AAV8), whereas moderate yields (25-44%) were obtained when only a 10-to-50 fold increase was registered (AAV1 and AAV9); finally, the lowest yield, obtained with AAV5, coincided with a mere 3-fold increase in K_{D}. A similar comparison can be made for the second best sequence CYIHFSGYTNYNPSLKSC (A1; SEQ ID NO: 1), whose high yield of AAV1, AAV2, and AAV6 coincided respectively with a 400-, 130-, and 2080-fold increase in K_{D}, while the product loss and lower yield registered with AAV8 and AAV9 coincided with both a lower binding strength and a lower shift in K_{D}. Shifts in affinity of such magnitude under mild acidification (pH 6) are hardly attainable with protein ligands, which require much harsher environments to release the bound capsids, and justify the choice of peptide ligands for the purification of labile products such as viral vectors. Secondly, peptides A3, A6, and A12 behaved differently with different serotypes, affording excellent capture and yield of AAV2 and AAV6, but high product loss and consequently low yield of AAV1, AAV5, AAV8, and AAV9. Notably, the low capture of these serotypes is matched by the in silico results, which predicted a low binding strength of these peptides at pH 7.4 (K_{D}>3.5 10-5 M); conversely, a higher binding strength (K_{D}~7.5 10⁻⁷ M-2.8 10⁻⁶ M) was predicted for AAV2 and AAV6, suggesting that, while moderate affinity is still desirable, binding strength must be above a minimum threshold to ensure sufficient product capture. On the other hand, the serotypes that were poorly captured by A3, A6, and A12 were effectively recovered using A1 and A4, thus prompting the choice of discontinue these peptides. Thirdly, sequence CVIDGSQSTDDDKIC (A10; SEQ ID NO: 10) performed uniquely well with AAV9, on par with AAVX POROS™ resin, while outperforming all other peptide-based resins and AVB Sepharose; once again, the performance of the peptide is inscribed in the values of binding strength at pH 7.4 (K_{D}~3.08·10⁻⁶ M) and strong affinity loss (220-fold increase in K_{D}) upon acidification predicted in silico. Under the light of these results, peptides A1, A4, and A10 were carried forward for additional experimental evaluation.

### Example 3

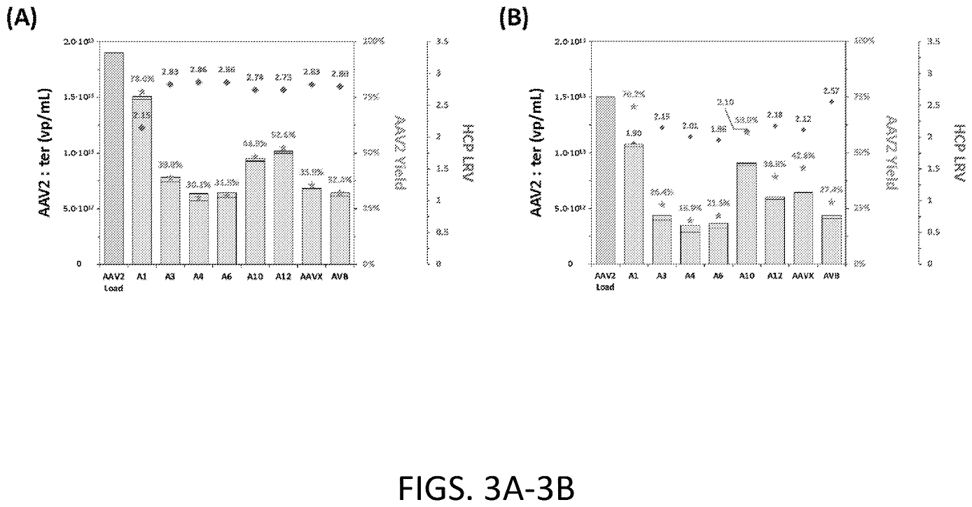

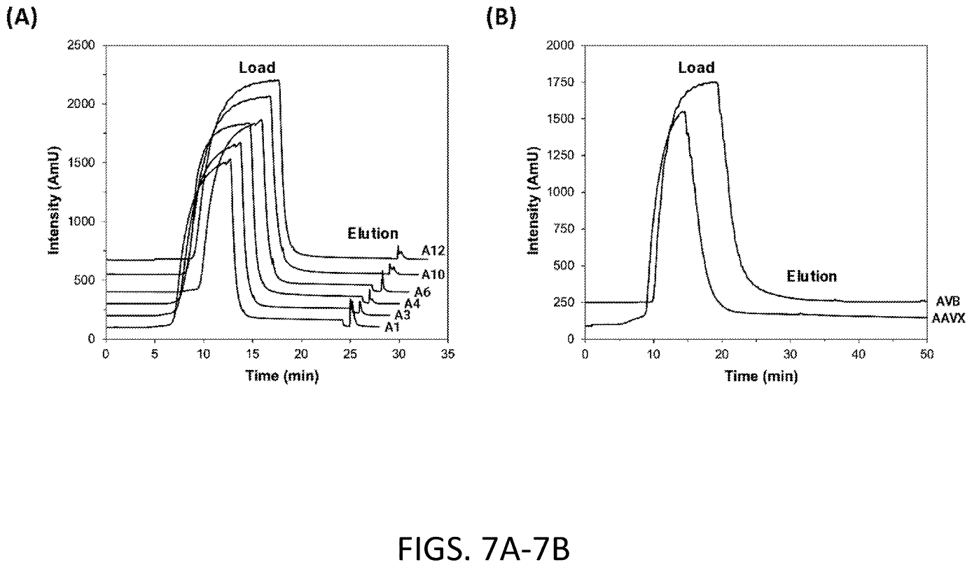

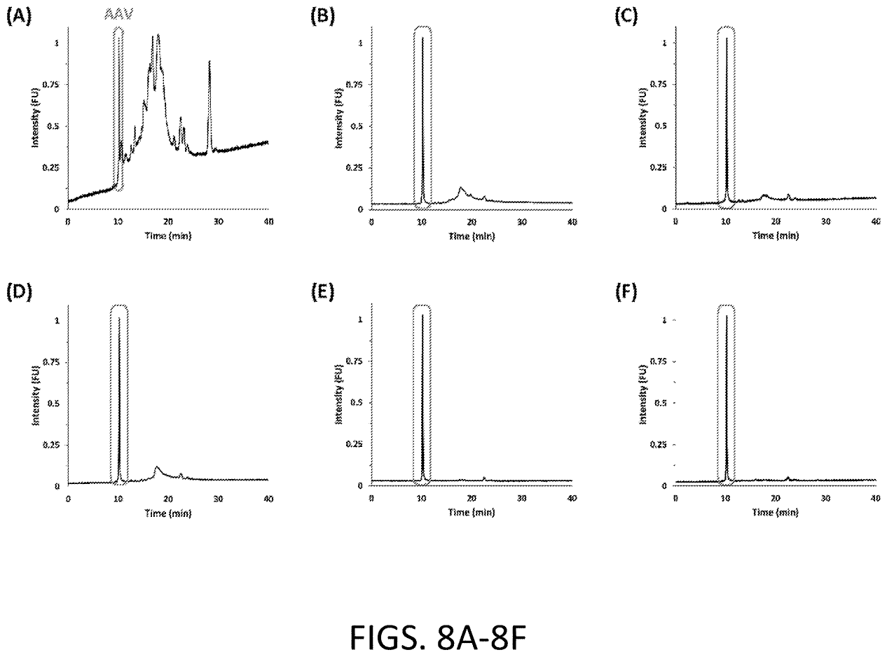

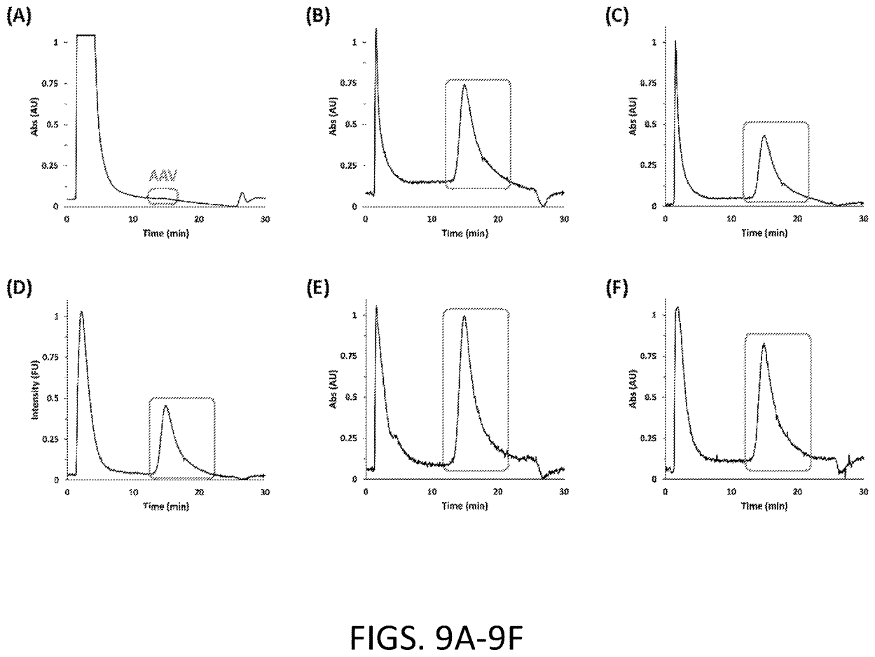

Purification of AAV2 from HEK293 and Sf9 cell lysates. Experiments were conducted to evaluate the three selected peptide-based adsorbents A1-, A4-, and A10-Toyopearl resin by purifying AAV2 from a clarified HEK293 cell culture lysate. The two feedstocks utilized—namely a HEK293 cell lysate featuring an AAV2 titer ~1.9·10¹² vp/mL and an HCP titer ~0.3 mg/mL, and a Sf9 cell lysate containing AAV2 at ~1.56·10¹² vp/mL and HCPs at ~1.1 mg/ml—are representative of bioreactor harvests in the gene therapy industry; similarly, a residence time (RT) of 3 mins for both binding and washing steps was adopted based on industrial operating conditions. The chromatograms of AAV2 purification are presented in FIG. 7, while the analysis of the collected fractions via size exclusion (SEC) and steric exclusion chromatography (SXC) are reported in FIGS. 8 and 9. Finally, the resulting values of AAV2 yield and logarithmic removal of HEK293 host cell proteins (HCP LRV) from the HEK293 and Sf9 feedstocks are summarized in FIGS. 3A and 3B, respectively.

The results of AAV2 purification from the HEK293 cell lysate (FIG. 3A) mirror the corresponding values obtained in non-competitive conditions in FIG. 2B: (i) little-to-no product loss was observed in the flow-through and wash fractions, confirming that the peptides maintain a strong AAV biorecognition when loaded with complex feedstocks; (ii) the product yields, ranging between 30% (A4- and A6-Toyopearl resins) and 78% (A1-Toyopearl resins), were either on par with or substantially higher than those returned by the reference POROS™ CaptureSelect™ AAVX Affinity and AVB Sepharose HP resins; and (iii) the values of HCP LRV were consistently above 2, reaching values as high as 2.86, corresponding to a >720-fold decrease of protein contaminants, thus matching the reference adsorbents in terms of product purity as well. The analysis of the feedstock and elution fractions via size exclusion chromatography (SEC, FIG. 8) and steric exclusion chromatography (SXC, FIG. 9) offer an at-a-glance of the AAV2 purification performance of the selected resins. While HEK293 ELISA assays returns the titer of HCPs only, analytical chromatography provides a quantitative measure of all process-related impurities—including denatured or hydrolyzed HCPs, other non-proteinaceous metabolites, host cell DNA and RNA, media components—as well as product-related impurities—such as capsid fragments. Mirroring the ELISA results, the SEC and SXC results demonstrate the high purity of the AAV2 eluted from the peptide-based adsorbents. Specifically, the comparative analysis of the SEC chromatograms returns global values of impurity decrease of 150-fold for A1-Toyopearl resin, 730-fold for A4-Toyopearl resin, and 550-fold for A10-Toyopearl resin. Similar results were provided by the SXC chromatograms, confirming that peptide-based adsorbents deliver eluates whose purity is comparable to that afforded by the affinity adsorbents utilized in the gene therapy industry.

Excellent results were also obtained on the front of AAV2 purification from the Sf9 cell lysate (FIG. 3B). As observed before, A1-Toyopearl resin afforded the highest product yield (70%), although the purity of the eluate was somewhat wanting, whereas A4- and A10-Toyopearl resin performed comparably to the control resins. While it cannot be excluded that the association of some HCPs to the AAV2 capsids may be responsible for the lower purity of the A1 eluates (note: the titer of HEK293 and Sf9 HCPs in the A1 vs. AAVX eluates differ by 9.7 and 13.7 μg/mL, respectively, corresponding to 2.7 and 1.2% of the HCPs in the feedstocks), it was thought that A1—as a small peptide ligand—may not quite match the binding selectivity of its V_{H}H counterparts. This can be alleviated by decreasing its density on the surface of the resin by further optimizing its sequence, which will be the object of future studies. It was also noted that, unlike the HEK293 cell lysate, the Sf9 harvest features a significantly higher HCP titer (0.3 vs. 1.1 mg/mL, respectively), which motivates why the values of HCP LRV are lower than those reported in FIG. 3A. Nonetheless, the concentration of residual HCPs in the eluates from the peptide-based adsorbents were consistently below 35 μg/mL, in line with eluates of affinity resins characteristic of chromatographic processes for biotherapeutics.

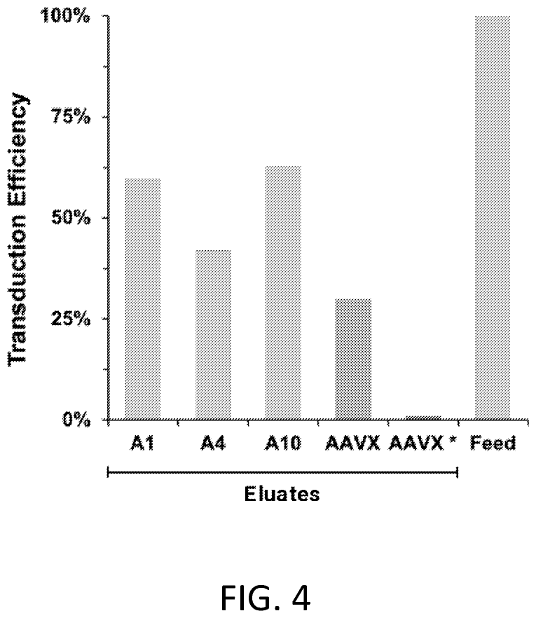

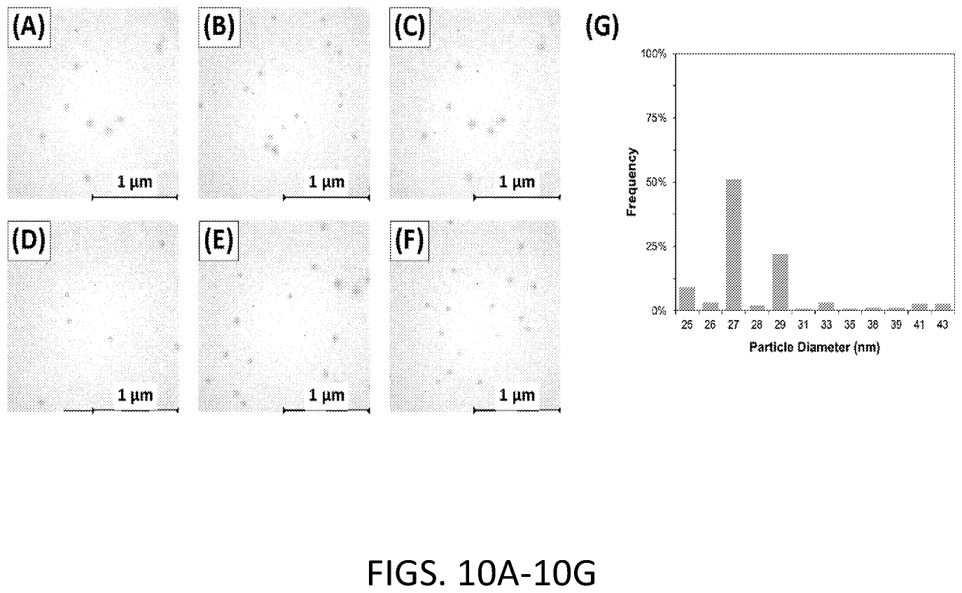

The purification performance of the peptide-based adsorbents is particularly remarkable when considering that elution is conducted under near-physiological pH. To quantify the benefits of mild elution, a comparative measurement of the transduction activity on human epithelial cells (HT1080) of the AAV2 isolated from HEK293 cell culture lysate was conducted using A1-, A4-, and A10-Toyopearl resins vs. POROS™ CaptureSelect™ AAVX resin (note: the gene encapsidated in the target AAV2 encodes for green fluorescence protein (GFP), which enables quantifying the transduction activity via fluorescence flow cytometry). The transduction activity—namely, the ability of a virus to effectively deliver its gene payload to the target cells—is a critical quality parameter of viral vectors and is significantly impacted by the process parameters. AAVs, like most viral vectors, are prone to lose their activity in response to variations in buffer conductivity and pH used to control adsorption and elution during chromatographic purification. Current affinity resins, however, require a rather acidic elution pH, in the range ~2-3 depending upon serotype and desired elution yield (>50%), to achieve sufficient product yield. By enabling elution under significantly milder conditions (1M MgCl₂ in the 20 mM Bis-Tris buffer at pH 6.0), the proposed peptide-based adsorbents are expected to return products with higher transduction activity than their commercial counterparts. The values collated in FIG. 4 confirm this hypothesis, showing that the activity of AAV2 eluted at pH 6 is between 1.5- and 2.2-fold higher than those isolated by the POROS™ AAVX resin. Remarkable is the activity of AAV2 purified by A1- and A10-Toyopearl resins, which, at 59% and 62% transduction relative to the AAV2 in the feedstock, match the values obtained via CsCl and iodixanol gradient ultracentrifugation. In this context, it was noted that the peptide-based resins release mostly intact and gene-loaded AAV2 particles, showing no signs of capsid fragmentation or aggregation (FIG. 10).

### Example 4

Dynamic AAV binding capacity and reusability of peptide-based adsorbents in competitive mode. Two additional performance parameters, which—together with the values of yield, purity, and activity of the recovered AAVs—determine the suitability of an affinity resin for industrial biopharmaceutical manufacturing are the dynamic binding capacity at 10% product breakthrough (DBC_{10%}) and the reusability upon subsequent cleaning in place. Accordingly, the DBC_{10%} of (A1; SEQ ID NO: 1) CYIHFSGYTNYNPSLKS-, (A4; SEQ ID NO: 4) CYGHFSGYGNYGPC-, and (A10; SEQ ID NO: 10) CVIDGSQSTDDDKIC-Toyopearl resins were measured via frontal loading of a clarified lysate containing AAV2 at the titer of 2.51·10¹² vp/mL at the residence time (RT) of 3 min (note: the clarified lysate was adopted in lieu of a pure AAV2 solution to provide a realistic evaluation of the binding capacity of the resins, whose operation is intended for competitive conditions; the adopted RT is recommended for POROS™ AAVX and AVB Sepharose resins and was therefore adopted to ensure comparability). The values of DBC_{10%}, compared in Table 7, demonstrate that the peptide-based adsorbents, notwithstanding the milder binding strength, feature an AAV binding capacity on par with or exceeding that of commercial affinity resins. This can be ascribed to the multi-site interaction governing the AAV adsorption by the peptide-functionalized surface, whose binding strength effectively matches that of protein-functionalized adsorbents. This decouples the value of binding capacity from the single AAV: peptide binding strength and makes it mostly- or solely-dependent on the specific surface of the resin.

Secondly, the binding capacity and selectivity of an affinity resin can decrease over time due to several factors, such as chemical degradation, physical damage, and fouling, leading to a loss of product yield and purity as well as additional costs related to the replacement and validation of the adsorbent. Unlike Protein A-based resins for antibody purification, whose lifetime has now reached 150-200 cycles with intermediate caustic cleaning in place, the commercial affinity resins for AAV purification cannot withstand harsh alkaline treatment and rapidly lose their binding capacity, mandating frequent column replacement. The reusability of the peptide-based adsorbents to withstand 20 cycles of AAV2 purification followed by regeneration and cleaning in place was evaluated. As shown in Table 7, A1—, A4, and A10-Toyopearl resins consistently maintained their DBC_{10%} (~10% variation), demonstrating the chemical stability of the selected peptides and their linkage to the resin.

TABLE 7
Values of dynamic AAV2 binding capacity (DBC_{10%})
of peptide-functionalized resins loaded (RT: 3 min)
with a clarified HEK293 cell lysate containing AAV2 at
the titer of 2.51 · 10¹² vp/mL.

DBC_{10%} (vp AAV per
mL of resin)

| Resin | Cycle 1 | Cycle 20 |
|---|---|---|
| A1-Toyopearl | 2.80 · 10¹⁴ | 2.35 · 10¹⁴ |
| A4-Toyopearl | 2.52 · 10¹⁴ | 2.16 · 10¹⁴ |
| A10 -Toyopearl | 2.10 · 10¹⁴ | 2.01 · 10¹⁴ |
| POROS ™ | 5.60 · 10¹⁴ | — |
| AAVX | | |

—: not available.

Viral vectors are rapidly becoming—and will soon be—an integral part of modern medicine: as the discourse on biomanufacturing evolves (e.g., the layout of platform processes, whether scaling-up vs. scaling-out will meet the growing demand, or the standardization and comparability in the process analytical technology, etc.), the need of a portfolio of bioprocess technologies dedicated to the expression, purification, and analytical characterization of viral vectors becomes every day more evident. Contributing to the efforts on improving downstream technologies, embodiments of the present disclosure provide an ensemble of small peptide affinity ligands designed to transform AAV purification as they provide (i) selective as well as flexible product capture, being serotype-agnostic and applicable to both HEK293 and Sf9 fluids; (ii) gentle elution, allowing product release under near-physiological pH; and (iii) robust reusability, maintaining a high binding capacity over multiple purification cycles. Furthermore, unlike affinity adsorbents that rely on antibody-derived ligands, the proposed adsorbents are undoubtedly more scalable and affordable, and they leverage the ability to mass manufacture GMP-quality peptides at relatively low cost (~US$8 per gram per amino acid residue, when manufactured at >10 kg scale per year). Combining the cost of synthesis with the average values of number of residues (~15-17) and molecular weight (~1.5-1.7 kg per mol) of the peptide ligands, their density on the resin surface (~0.03 mol per liter), and the cost of the base resin (~$2,500 per liter) indicates that direct material cost of the peptide-functionalized adsorbent ranges between $7,900 and $9,500 per liter, when produced at the ~100 liters scale (note: direct labor and manufacturing overhead are not factored).

These considerations, combined with the purification performance of peptide-functionalized adsorbents, show the promise of this technology to transform the biomanufacturing of modern medicines and reduce their cost (note: the latter is of particular concern, given the price tag of gene therapies well above US$1M per patient). Under these considerations, experiments were conducted to (i) demonstrate further the technology introduced in the present disclosure by purifying AAVs of different serotypes from a variety of HEK293 and Sf9 fluids; and (ii) leverage the in silico-in vitro toolbox for ligand development to establish a portfolio of peptide-based purification tools for the other key viral vector families, namely lentivirus (LV), adenovirus (Ad), and baculovirus (BV).

TABLE 8
Pairwise interactions between the active residues on AAVR and the targeted
residues on the virion protein (VP1) of AAV1 (PDB ID: 6JCQ), AAV2 (6IHB),
AAV5 (6JCS), and AAV9 (7WJX) as well as between the active residues on
A20 and the targeted residues on the virion protein (VP1) of AAV2 (3J1S).

| | ΔG | | | ΔG | | | ΔG |
|---|---|---|---|---|---|---|---|
| AAVR | (kcal/mol) | AAV1 | AAVR | (kcal/mol) | AAV2 | AAVR | (kcal/mol) |
| SER413 | 0.17 | THR504 | ASN145 | 0.093 | GLN385 | VAL305 | 0.175 |
| | | ASN500 | SER153 | 0.558 | THR503 | LEU348 | 0.175 |
| VAL427 | 0.43 | THR504 | | | GLU499 | ILE349 | 0.524 |
| | | SER499 | PRO154 | 0.279 | GLU499 | | |
| ASP429 | 0.77 | THR504 | VAL167 | 0.372 | THR503 | | |
| | | TRP503 | ILE168 | 0.093 | THR503 | THR350 | 0.873 |
| SER431 | 0.60 | TRP503 | ASP169 | 0.93 | TRP502 | | |
| | | ASN269 | | | THR503 | | |
| | | ASP270 | | | LYS507 | | |
| | | SER268 | SER171 | 0.651 | LYS507 | | |
| GLN432 | 0.43 | TRP503 | | | ASN268 | HIS351 | 0.873 |
| | | ASP270 | | | ASP269 | | |
| | | SER268 | | | TRP502 | | |
| | | ASN271 | | | SER267 | | |
| SER 433 | 0.86 | ASN269 | GLN172 | 0.186 | TRP502 | | |
| | | SER268 | | | SER267 | | |
| | | ALA267 | SER173 | 0.93 | ALA266 | | |
| THR434 | 0.60 | GLY266 | | | SER267 | PRO352 | 0.873 |
| | | ALA267 | | | ASN268 | | |
| | | ASN269 | THR174 | 0.558 | SER267 | | |
| | | SER268 | | | ALA266 | | |
| ASP435 | 0.77 | HIS272 | | | GLY265 | | |
| | | SER268 | ASP175 | 0.837 | ALA266 | ARG353 | 0.611 |
| | | SER262 | | | HIS271 | | |
| | | GLY266 | | | SER384 | | |
| | | ALA267 | | | SER264 | | |
| ASP436 | 0.43 | SER385 | | | GLY265 | | |
| | | HIS272 | | | GLN263 | | |
| | | ALA267 | | | SER267 | | |
| | | SER262 | | | SER262 | | |
| | | ALA263 | ASP176 | 0.651 | GLY265 | | |
| | | GLY266 | | | HIS271 | | |
| ASP437 | 0.69 | ASN269 | | | SER264 | | |
| | | HIS272 | | | GLY383 | ASP354 | 0.262 |
| | | ALA267 | | | GLN263 | | |
| | | GLN386 | | | GLN385 | | |
| | | GLY384 | | | SER262 | | |
| | | SER385 | | | ALA266 | TYR355 | 0.873 |
| | | ASN383 | | | SER384 | | |
| LYS438 | 0.51 | GLY384 | ASP177 | 0.837 | HIS271 | SER356 | 0.611 |
| | | ASN512 | | | GLY383 | | |
| | | SER385 | | | ASN382 | | |
| | | ASN383 | | | GLN385 | | |
| | | ASN269 | | | SER384 | LYS371 | 0.175 |
| | | HIS272 | | | SER262 | | |
| | | GLY513 | | | ALA266 | PRO374 | 0.262 |
| ILE439 | 0.69 | ASN269 | LYS178 | 0.558 | ASN511 | | |
| TYR442 | 0.51 | ASN269 | | | GLY383 | GLY375 | 0.873 |
| ILE462 | 0.34 | LYS508 | | | ASN382 | | |
| | | TRP503 | | | HIS271 | | |
| | | THR504 | | | SER384 | LEU376 | 0.873 |
| | | | | | ASN268 | | |
| | | | ILE179 | 0.93 | SER267 | | |
| | | | | | ALA266 | | |
| | | | | | ASN268 | | |
| | | | | | HIS271 | GLU378 | 0.524 |
| | | | TYR182 | 0.372 | ASN268 | | |
| | | | ILE202 | 0.465 | THR503 | | |
| | | | | | LYS507 | THR397 | 0.524 |
| | | | | | TRP502 | VAL398 | 0.960 |
| | | | | | | LYS399 | 0.087 |
| | | | | | | PRO400 | 0.175 |

| | | ΔG | | | | ΔG | |
|---|---|---|---|---|---|---|---|
| AAV5 | AAVR | (kcal/mol) | AAV9 | A20-H | A20-L | (kcal/mol) | AAV [symbol] |
| SER319 | ARG406 | 0.284 | GLY266 | | TYR91 | 0.669 | THR716 |
| GLU708 | | | ASN262 | | | | ASN717 |
| GLN532 | | | THR264 | | TRP92 | 2.008 | THR716 |
| PRO533 | | | SER263 | | | | ASN717 |
| ASN535 | | | GLY267 | | ILE93 | 3.346 | ASN717 |
| GLN532 | SER413 | 0.213 | GLU500 | | | | VAL719 |
| TYR542 | GLN417 | 0.496 | SER499 | | GLY94 | 2.008 | ASN717 |
| ALA534 | GLU418 | 0.071 | ASN498 | | PHE96 | 0.669 | ASN717 |
| ASN530 | VAL427 | 0.425 | GLU500 | TYR52 | | 1.367 | THR716 |
| ASN546 | | | PRO504 | HIS54 | | 1.708 | VAL708 |
| ASP704 | ASP429 | 0.638 | PRO504 | PHE55 | | 1.025 | VAL708 |
| SER531 | | | TRP503 | | | | LYS706 |
| ASN546 | SER431 | 0.567 | SER269 | SER56 | | 1.025 | VAL708 |
| GLY545 | | | TRP503 | TYR58 | | 0.683 | VAL708 |
| GLN532 | | | ASN270 | | | | ASN709 |
| GLU708 | | | ASP271 | PRO63 | | 0.683 | GLU54 [symbol] |
| ARG710 | GLN432 | 0.142 | TRP503 | LYS66 | | 1.367 | LYS55 [symbol] |
| SER531 | | | SER269 | SER67 | | 0.342 | ASP55 [symbol] |
| ASN546 | | | SER268 | | | | |
| GLY545 | SER433 | 0.709 | SER269 | | | | |
| GLN532 | | | ASN270 | | | | |
| ARG710 | THR434 | 0.425 | SER268 | | | | |
| ARG710 | | | SER269 | | | | |
| PHE698 | | | GLY266 | | | | |
| MET547 | | | GLY267 | | | | |
| SER531 | ASP435 | 0.709 | GLY266 | | | | |
| THR711 | | | GLY267 | | | | |
| ASN546 | | | ASN262 | | | | |
| GLU544 | | | SER268 | | | | |
| THR712 | | | SER269 | | | | |
| LEU548 | | | ASN270 | | | | |
| GLY545 | | | SER263 | | | | |
| ARG713 | ASP436 | 0.567 | SER386 | | | | |
| THR712 | | | GLY266 | | | | |
| GLY545 | | | SER263 | | | | |
| ARG710 | | | GLY267 | | | | |
| PHE698 | | | SER268 | | | | |
| PHE698 | | | ASN262 | | | | |
| ARG710 | ASP437 | 0.638 | ALA273 | | | | |
| ARG710 | | | SER386 | | | | |
| ASP704 | | | ASN270 | | | | |
| GLN697 | | | GLN387 | | | | |
| PHE698 | | | SER263 | | | | |
| GLN697 | | | ASP384 | | | | |
| GLY545 | | | SER269 | | | | |
| ASN546 | | | ASN262 | | | | |
| LEU543 | | | SER268 | | | | |
| ASN546 | | | GLY385 | | | | |
| LEU543 | LYS438 | 0.425 | SER386 | | | | |
| GLY545 | | | GLY385 | | | | |
| TYR542 | | | ASP384 | | | | |
| THR541 | | | ASN262 | | | | |
| ALA540 | | | ALA273 | | | | |
| ASN546 | | | ASN270 | | | | |
| LEU543 | ILE439 | 0.638 | ASN270 | | | | |
| THR538 | VAL440 | 0.071 | ASN270 | | | | |
| TYR542 | TYR442 | 0.425 | SER269 | | | | |
| ALA540 | | | ASN270 | | | | |
| LEU543 | ILE462 | 0.284 | PRO504 | | | | |
| LEU543 | | | TRP503 | | | | |
| LEU543 | LYS464 | 0.071 | PRO504 | | | | |
| GLY478 | | | | | | | |

[symbol]

indicates data missing or illegible when filed

TABLE 9
Sequences and biophysical properties of selected AAVR-mimetic and A20-mimetic
AAV-binding peptide ligands. The values of isoelectric point (pI), polarity
(Grantham scale), and Grand Average Hydropathy index (GRAVY) were calculated
based on the amino acid sequence and assuming an amidated C-terminus to represent
the conjugation of the peptide to the chromatographic resin.

| AAVR mimetic | | | | A20 mimetic | | | |
|---|---|---|---|---|---|---|---|
| Sequence | pI | Polarity | GRAVY | Sequence | pI | Polarity | GRAVY |
| VIDGSQSTDDDKI | 3.83 | 0.55 | −0.393 | YIHFSGYTNYNPSLKS | 8.94 | −0.64 | −0.388 |
| (SEQ ID NO: 10) | | | | (SEQ ID NO: 1) | | | |
| DGSQSTDDDKI | 3.83 | 0.89 | −1.123 | YIHFSGYTNYNGSLKS | 8.94 | −0.64 | −0.322 |
| (SEQ ID NO: 11) | | | | (SEQ ID NO: 2) | | | |
| DSQSTDDDKI | 3.83 | 0.97 | −1.183 | YVHFSGYSNYSPS | 8.24 | −0.80 | −0.253 |
| (SEQ ID NO: 12) | | | | (SEQ ID NO: 3) | | | |
| SGSTDDDKI | 4.25 | 0.76 | −0.691 | YGHFSGYGNYGP | 8.24 | −0.80 | −0.485 |
| (SEQ ID NO: 13) | | | | (SEQ ID NO: 4) | | | |
| GYWIGPFTGGGYIHFSGYT | 9.36 | -1.05 | 0.021 | YGHFSPYGNYGP | 8.24 | −0.80 | −0.571 |
| (SEQ ID NO: 14) | | | | (SEQ ID NO: 5) | | | |
| GYWIGPFTGPGYIHFSGYT | 9.36 | -1.05 | −0.042 | YIHFSGYTNYNP | 8.24 | −0.96 | −0.388 |
| (SEQ ID NO: 15) | | | | (SEQ ID NO: 6) | | | |
| GYWIGPFTPGPYIHFSGYT | 9.36 | -1.05 | −0.105 | YHFSYNYPKS | 8.94 | −0.68 | −0.825 |
| (SEQ ID NO: 16) | | | | (SEQ ID NO: 8) | | | |
| LITHPRDYSPKLTPGLYEFG | 9.44 | -0.16 | −0.515 | YHFSYNYP | 8.24 | −1.14 | −0.520 |
| | | | | (SEQ ID NO: 9) | | | |

### Example 5

Serotype-agnostic AAV binding and release by AAVR-mimetic peptide ligands. As described herein, the A20- and AAVR-mimetic peptides were designed to target residues that are displayed by contiguous VPs and are conserved across serotypes. Peptides were cyclized to enhance their binding affinity and selectivity by reducing the entropic penalty of the binding energy. Additionally, single amino acid mutations were introduced in the A20-mimetic peptides to expand their targeting beyond AAV2, although that did not manage to reach the pan-selective biorecognition of their AAVR-mimetic counterparts. Initial characterization of A20-mimetic CYIHFSGYTNYNPSLKSC (A1; SEQ ID NO: 1), CYVHFSGYSNYSPSC (A3; SEQ ID NO: 3), and CYGHFSGYGNYGPC (A4; SEQ ID NO: 4), and AAVR-mimetic CVIDGSQSTDDDKIC (A10; SEQ ID NO: 10) and its shorter variants CDGSQSTDDDKIC (A11; SEQ ID NO: 11), CDSQSTDDDKIC (A12; SEQ ID NO: 12), and CSGSTDDDKIC (A13; SEQ ID NO: 13) corroborated the design criteria, but was not conducted systematically on all AAV serotypes. Accordingly, this example demonstrates the ability of these peptide ligands to (i) capture and purify AAVs in a serotype-agnostic fashion from complex feedstocks and (ii) withstand multiple purification cycles with intermediate caustic cleaning in place.

Adeno-associated viruses (AAVs) have emerged as a central family of vectors in the realm of gene delivery, providing therapeutic options to diseases once deemed incurable. At the same time, they necessitate efficient and affordable purification methods that can be platformed to serve all AAV serotypes. Current chromatographic tools, while affording high product purity, fail to bind certain serotypes, provide limited yield and lifetime, and impose harsh elution conditions that can compromise the vector's activity and safety. Embodiments of the present disclosure address these challenges by exploring the potential of peptide ligands discovered for serotype-agnostic AAV purification via affinity chromatography. Previous studies reveal a pH-dependent affinity interaction: AAV2, AAV3, AAV6, AAV9, and AAVrh.10 are effectively captured at neutral pH, while binding AAV1, AAV5, AAV7, and AAV8 is stronger in acidic environment (pH 5). The elution of bound AAVs was achieved using magnesium chloride at neutral pH for all serotypes, consistently affording capsid yields above 50% and genome yields above 80%, together with a >100-fold reduction in host cell proteins and nucleic acids. In particular, peptide ligand A10 exhibited remarkable binding capacity (up to 5·10¹⁴ vp per mL of resin) and purification performance for all AAV serotypes, demonstrating a clear promise for industrial AAV manufacturing. Finally, alkaline-stable variants of A10 were developed and utilized to conduct multiple cycles of AAV2, AAV5, AAV8, and AAV9 purification with intermediate caustic cleaning without loss of capacity, product yield, or purity. Collectively, these results demonstrate the promise of this technology to further the impact and affordability of gene therapy.

The AAV binding to cell-displayed AAVR varies among serotypes in a pH-dependent fashion. These differences, rooted in the amino acid sequence of the virion proteins (VPs), are manifested in the structural and functional features unique to each serotype, and govern AAV's response to the environment, tissue tropism, and cell interaction. For example, the N-terminus of VP1 undergoes a reversible pH-induced unfolding and refolding process, while the N-termini of VP3 and VP2 are reversibly externalized as the pH is lowered from 7.4 to 5. These mechanisms result in variations in the AAV:AAVR interaction that favor certain serotypes at neutral pH and others in acidic environment. The impact of these phenomena reaches beyond AAV infectivity and becomes evident in AAV purification—whether by ion-exchange or affinity chromatography. Accordingly, experiments were conducted to evaluate the A10, A11, A12, and A13 binding of AAV serotypes 1, 2, 3, 5, 6, 7, 8, 9, and rh. 10 non-competitive conditions at both pH 5 and 7.4 to identify optimal adsorption conditions to be utilized in the subsequent AAV purification studies. All feedstocks were adjusted to an AAV titer of ~5·10¹² vp/mL, which resembles the concentration of cell lysates found in the gene therapy industry, and loaded on the resins at ~5·10¹³ vp per mL of resin. The AAVs bound at neutral pH were eluted from the peptide-functionalized chromatographic resins at pH 6.5 (400 mM MgCl₂ in 10 mM Bis-Tris buffer), whereas the AAVs bound at pH 5 were initially eluted at pH 7 (20 mM NaCl and 2 mM MgCl₂ in 10 mM Bis-Tris buffer) followed by a second elution step at pH 6.5. The elution of AAVs at near-physiological pH is a unique feature of peptide ligands, whose multi-site binding through a dense network of hydrogen bonds provides for both high binding capacity and facile product release.

The results collated in Table 10 document the pH-governed binding activity of AAVR-mimetic peptide ligands. Specifically, AAV serotypes 2, 3, 6, 9, and rh.10 were effectively captured at neutral pH, with a less than 3% loss in the flow-through fraction, and were eluted with high yield (up to 70%; note: higher yields can be obtained by increasing the load to 10¹⁴ vp per mL of resin, as done in subsequent purification studies, vide infra). Notable exceptions were observed with AAV9, which was captured effectively by ligand A10 only, with a loss of 2.9% and a yield of 66.3%; and AAVrh.10, which was captured by A10 and A11, but not A12, despite the latter differing by one residue only. These results demonstrate the binding selectivity of the AAVR-mimetic peptides to the virion proteins: the formation of a network of hydrogen bonds sufficiently strong to capture AAVs, which are present in the feed at low titer (~5·10¹² vp/mL comprise ~10¹⁴ encapsidated VPs per mL, corresponding to ~0.2 μM).

Serotypes 1, 5, 7, and 8 were captured in acidic conditions, where they exhibited low values of loss in the flow-through (<4%). Notably, elution was conducted in two steps, the first at low conductivity and pH 7 and the second in 0.4 M MgCl₂ and pH 6.5. The first elution step afforded appreciable values of yield, yet insufficient in the context of bioprocessing, necessitating the second elution step to achieve global yields up to 90% (Table 10). The differential release of AAVs in buffers with different conductivities and pH results from the heterogeneity of the capsids (e.g., VP ratio and structural arrangement, identity of genetic payload, etc.). This suggests the applicability of peptide ligands not only for purifying, but also for fractionating capsids and isolating the population with higher transduction activity.

It is also worthy of note that AAV1 and AAV6, despite belonging to clade A and their VPs sharing 99% sequence and structural homology, require different binding pH. A similar effect was observed with AAV8 and AAVrh.10, which belong to clade E and share 93% sequence-based and 97% structural homology. This indicates, as observed above, that the peptide ligands target highly defined binding sites on the capsid surface, which require optimal display for effective AAV capture. Collectively, these results indicate that A10 can be utilized as a universal ligand for AAV purification, but that ad hoc chromatographic protocols are needed to achieve the productivity and product quality needed for the efficacy, safety, and affordability of gene therapies.

TABLE 10
Capsid loss and yield obtained by loading pure AAV1, AAV2,
AAV3, AAV5, AAV6, AAV7 AAV8, AAV9, and AAVrh.10
at the titer of ~5 · 10¹² vp/mL in 20 mM NaCl in 10 mM
Bis-Tris buffer at pH 7.0, or AAV1, AAV5, AAV7, and
AAV8 at the titer of ~5 · 10¹² vp/mL in 2 mM
MgCl₂ in 50 mM acetate buffer at pH 5.0 on A10-,
A11-, A12-, and A13-Toyopearl resins at the load
of ~5 · 10¹³ vp per mL of resin. The AAVs
bound at neutral pH were eluted using 400 mM
MgCl₂ in 10 mM Bis-Tris buffer at pH 6.5,
whereas the AAVs bound at pH 5 were eluted
in two steps using 20 mM NaCl and 2 mM
MgCl₂ in 10 mM Bis-Tris buffer at pH 7.0
and 400 mM MgCl₂ in 10 mM Bis-Tris
buffer at pH 6.5 (the reported yield
is the sum of the step yields).

| | | Binding at pH 7.0 | | Binding at pH 5.0 | |
|---|---|---|---|---|---|
| | | Capsid | Capsid | Capsid | Capsid |
| | | Yield | Loss | Yield | Loss |
| Serotype | Peptide | (%) | (%) | (%) | (%) |
| AAV1 | A10 | 3.6 | 100 | 88.5 | Undetectable |
| | A11 | 3.0 | 100 | 56.8 | |
| | A12 | 2.5 | 100 | 61.3 | |
| | A13 | 2.9 | 100 | 21.2 | |
| AAV2 | A10 | 44.6 | 0.10 | Not measured | |
| | A11 | 49.5 | 2.81 | | |
| | A12 | 51.7 | 1.38 | | |
| | A13 | 54.9 | 1.63 | | |
| AAV3 | A10 | 59.9 | 1.34 | Not measured | |
| | A11 | 53.9 | 2.53 | | |
| | A12 | 52.2 | 1.56 | | |
| | A13 | 48.6 | 7.65 | | |
| AAV5 | A10 | 4.1 | 94.4 | 62.9 | Undetectable |
| | A11 | 3.1 | 95.9 | 42.5 | |
| | A12 | 3.3 | 94.1 | 39.6 | |
| | A13 | 0.0 | 100 | 48.9 | |
| AAV6 | A10 | 52.8 | 1.26 | Not measured | |
| | A11 | 39.8 | 1.94 | | |
| | A12 | 34.0 | 1.54 | | |
| | A13 | 33.9 | 1.60 | | |
| AAV7 | A10 | | 76.3 | 49.4 | Undetectable |
| | A11 | | 82.1 | 42.9 | |
| | A12 | Undetectable | 75.4 | 46.3 | |
| | A13 | | 88.5 | 12.2 | 51.2 |
| AAV8 | A10 | 5.00 | 77.0 | 51.2 | ND |
| | A11 | 7.50 | 75.6 | 38.4 | |
| | A12 | 5.44 | 75.4 | 45.9 | |
| | A13 | 5.52 | 73.9 | 18.8 | 56.65 |
| AAV9 | A10 | 66.3 | 2.9 | Not measured | |
| | A11 | 6.1 | 80.0 | | |
| | A12 | 5.4 | 84.1 | | |
| | A13 | 5.9 | 86.5 | | |
| AAVrh.10 | A10 | 59.6 | 23.5 | Not measured | |
| | A11 | 55.1 | 36.6 | | |
| | A12 | 3.1 | 29.3 | | |
| | A13 | 51.1 | 9.3 | | |

### Example 6

Purification of AAV serotypes 1, 2, 3, 5, 6, 7, 9, and rh.10 from HEK293 cell lysates. The debate on the optimal AAV expression system has occupied decades, comparing the performance of HEK293 vs. Sf9 cells in terms of capsid titer, full/empty capsid ratio, in vitro, and in vivo potency. While some studies favor mammalian systems for their product quality while others advocate for insect cells for higher productivity, there is a general consensus in adopting HEK293 cells for small preparations dedicated to orphan diseases and clinical trials of investigational new gene therapies. Under such premise, all purification efforts conducted in the present disclosure utilized HEK293 cell culture lysate as model feedstocks.

The first set of purification tests focused on serotypes 2, 3, 6, 9, and rh.10, which enable adsorption at neutral pH. Experiments were conducted to compare the purification performance of AAVR-mimetic ligands with A20-mimetics A1, A3, A4, and A6, and commercial standards Poros™ CaptureSelect™ AAVX and AVB Sepharose HP affinity resins. After equilibration, the columns were loaded to a ratio of 10¹³ vp/mL, corresponding to ~10% of the binding capacity of the affinity resins. Following wash, the bound AAVs were eluted from all peptide-functionalized resins at pH 6.5, whereas elution from commercial resins required much harsher conditions (pH 2-2.5), as recommended by the manufacturers.

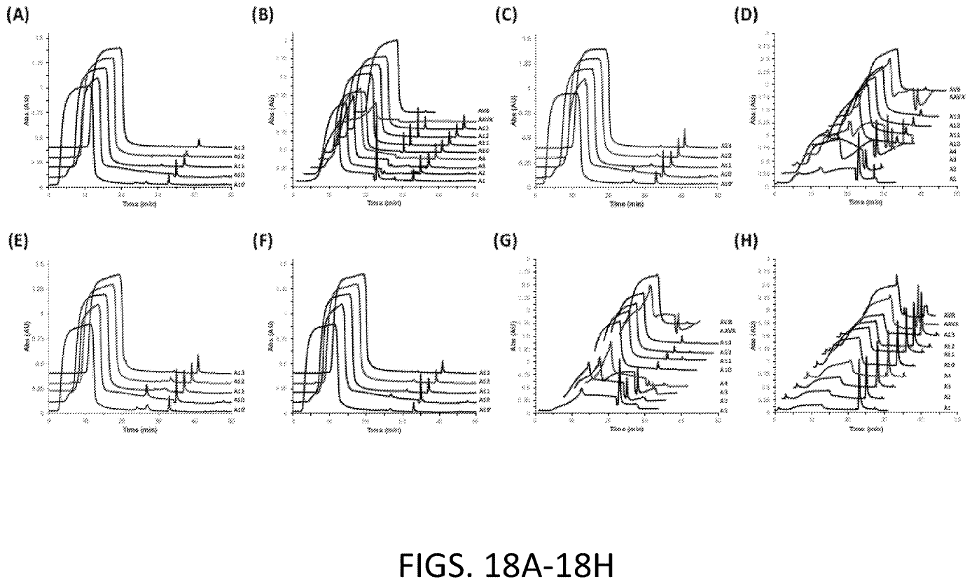

The chromatograms of AAV purification collated in FIGS. 18A-18D provide a visual representation of the separation dynamics, offering insights into the efficiency and effectiveness of the purification process. The flow-through segment of the various chromatograms indicate different adsorption dynamics, likely rooted in the material properties of the chromatographic substrates (i.e., polymethyl-methacrylate beads of 65 μm diameter and ~100 nm pores for the peptide-Toyopearl resin; polystyrene-divinylbenzene beads of 50 μm diameter and ~1000 nm pores for CaptureSelect™ AAVX resin; and crosslinked agarose beads of 35 μm diameter and ~30 nm pores) and AAV: ligand association kinetic. Conversely, the elution peaks are consistently sharp, suggesting—in tandem with the complementary analytics presented below—a rapid release of bound capsids.

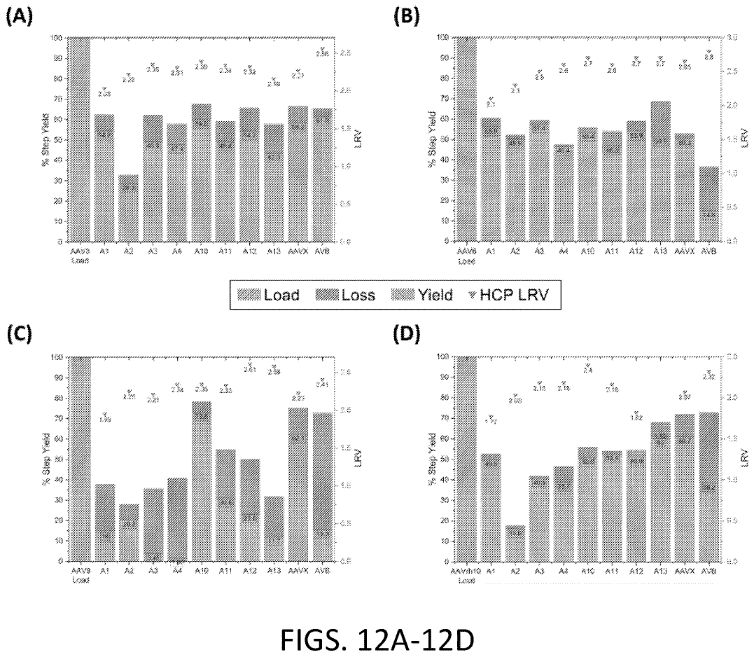

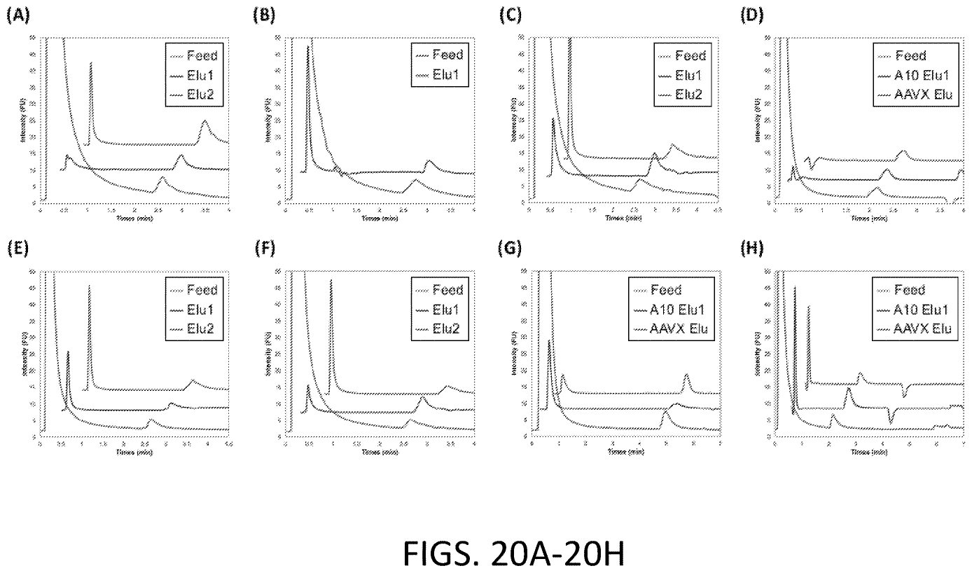

The collected fractions were analyzed by serotype-specific ELISA and q/ddPCR to quantify the capsid and transgene yields, and HEK293 HCP ELISA, size exclusion chromatography (SEC), and steric exclusion chromatography (SXC) to quantify the AAV purify. The AAVR-mimetic ligands consistently afforded yields of 50-to-60% and 400-to-500-fold reduction of HCPs, on par with AAVX and AVB affinity resins (FIGS. 12A-12D). The A20-mimetic peptides afforded comparable values of yields of serotypes 3, 6, and rh.10, but lower values of purity (200-to-300-fold reduction of HCPs). Ligand A10 emerged as a top performing ligand by providing a consistently high purification performance. Particularly remarkable was the AAV9 purification by A10, which outperformed all other ligands in terms of product yield and purity (72.8% and 230-fold reduction of HCPs). SEC and SXC data showed a significant increase in capsid titer (retention time ~10-10.5 min in FIGS. 20A-20H; retention time ~2-2.5 min in Figure S4) and confirmed the reduction in all biomolecular contaminants (~11-25 min in FIGS. 20A-20H; ~0.2-2 min in FIGS. 20A-20D).

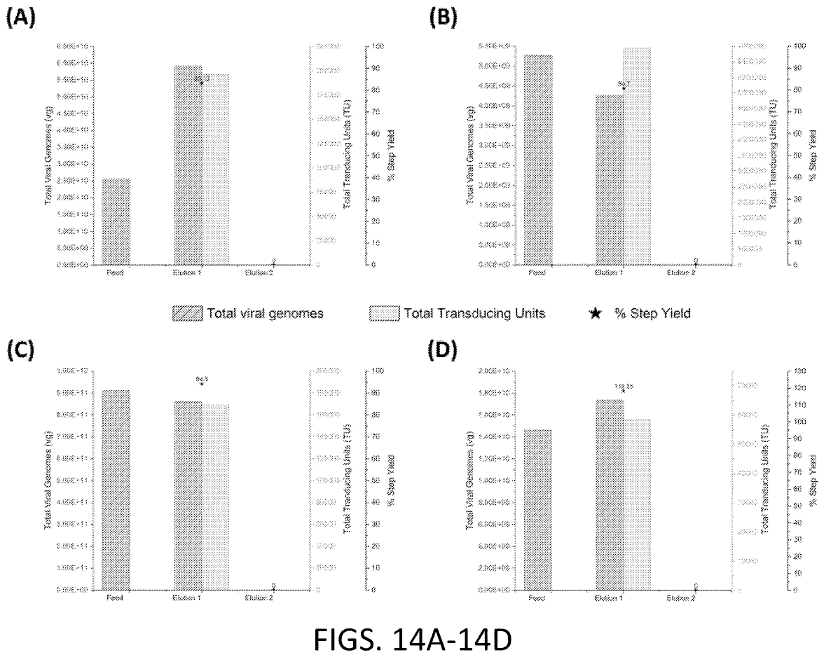

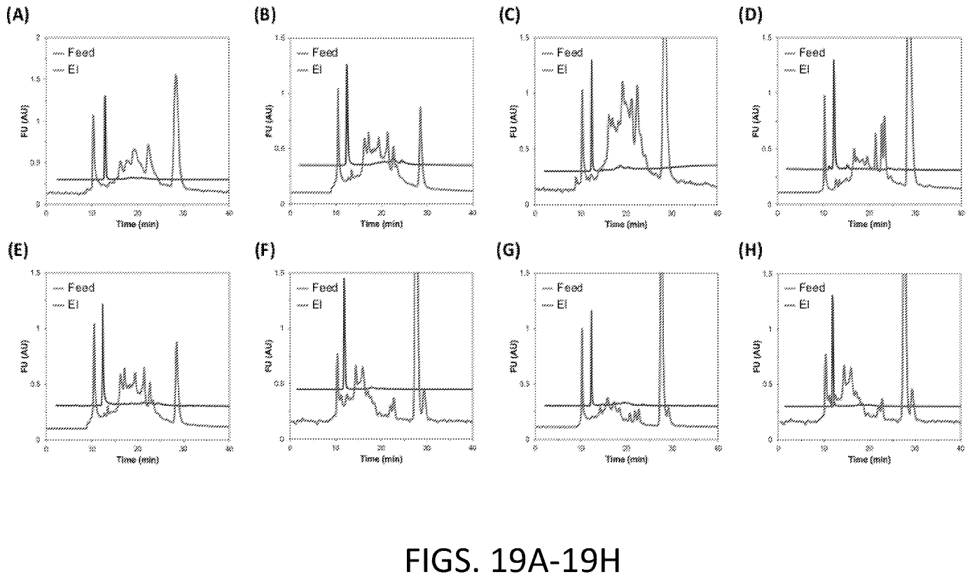

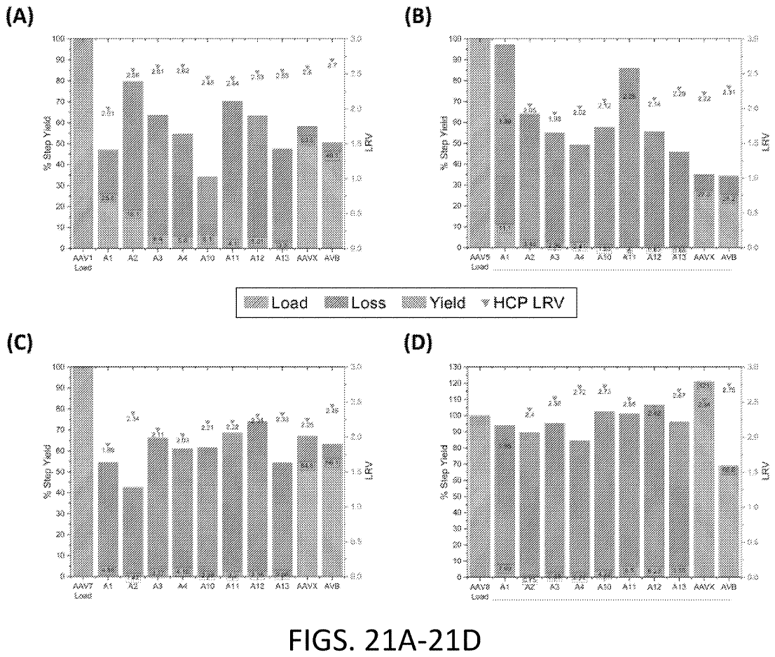

The peptides were unsuccessful in purifying AAV1, AAV5, AAV7, and AAV8 from HEK293 cell lysates at pH 7.4 (FIGS. 21A-21D). Informed by the binding studies in non-competitive conditions, experiments were conducted to purify these serotypes after conditioning the feedstocks to a conductivity of ~3 mS/cm and a pH of 5 via tangential flow filtration. The resulting chromatograms, collated in FIGS. 19A-19H, resemble those obtained with serotypes 2, 3, 6, 9, and rh. 10, suggesting that adjusting the pH of the binding buffer is sufficient to recapitulate the kinetics of affinity adsorption and release of the peptide ligands. This is confirmed by the values of capsid yields and HEK293 HCP removal summarized in FIGS. 14A-14D, which stood respectively at 50-60% and 200-to-500-fold for serotypes 1, 5, and 8—placing the purification performance of peptide ligands on par with that of AAVX (note: AVB failed to bind effectively serotypes 5, 6, 7, 9, for which the yield was limited to 15%). As noted with AAV9, peptide A10 afforded remarkable results with AAV7, outperforming all other ligand with a yield of 73% and a 250-fold reduction of HCPs. The SEC and SXC results in FIGS. 20A-20H and FIGS. 21A-21D confirm these finding by providing an at-a-glance demonstration of capsid concentration and clearance of all soluble contaminants across the entire spectrum of composition and size.

### Example 7

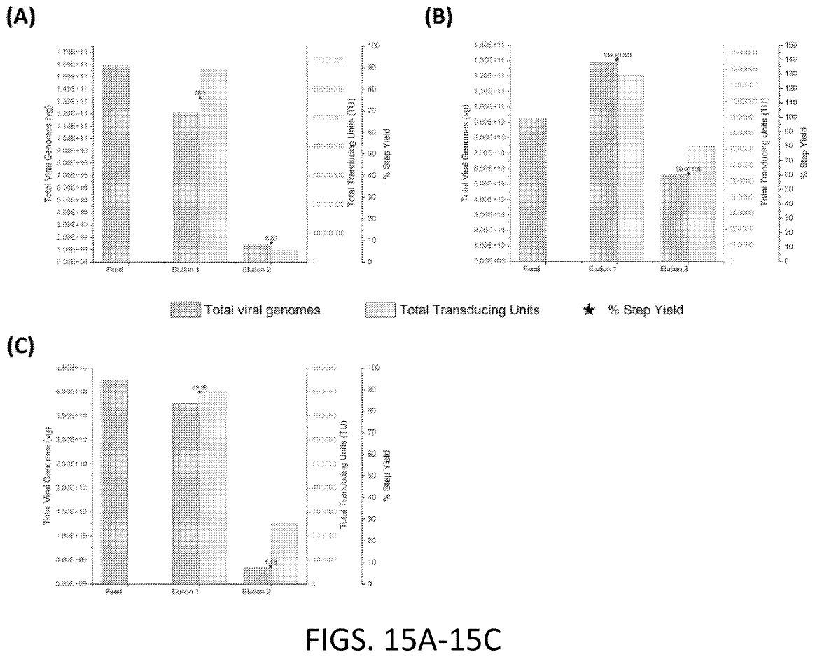

Optimizing AAV recovery and purity using AAVR-mimetic ligand A10. The first set of purification tests was conducted by loading the resin to a ratio of 10¹³ vp per mL of resin (due to limited availability of feedstock). Prior studies, however, indicated that higher resin loads and the combination of loading in up-flow and elution in down-flow is conducive to higher values of yield. The purification of all serotypes was therefore repeated using the lead ligand A10 by increasing the load to approach the dynamic binding capacity (DBC_{10%}~1-3·10¹⁴ vp per mL of resin). A10 was demonstrated to capture preferentially assembled AAV particles and release an early elution fraction enriched in transgene-loaded capsids, thus affording a concomitant enrichment and purification of transducing virions. Accordingly, the elution of bound viruses was conducted in two steps. The resulting values of yield of encapsidated genome and transducing virions are reported in FIGS. 14A-14D for serotypes 3, 6, 9, and rh.10 (bound at pH 7.4), and in FIGS. 15A-15C for serotypes 1, 7, and 8 (bound at pH 5).

The values of genome enrichment (elution vs. feed) of the serotypes bound in neutral pH, together with the high yield (80-100%) and HCP clearance (LRV ~2.3-2.7), confirms the excellent purification performance of A10. The high values of transduction activity of eluted AAVs, likely rooted in the moderate binding strength and mild elution conditions, confirm the ability of A10 to safeguard the potency of gene therapies (note: HT1080 cells were utilized in lieu of HEK293 or HeLa cells owing to their consistent performance in transducing different serotypes; the large amounts of host cell proteins in the cell lysates inhibited the expression of GFP and disrupted the cells in most cases, allowing to measure the transduction activity of AAV in the eluates only).

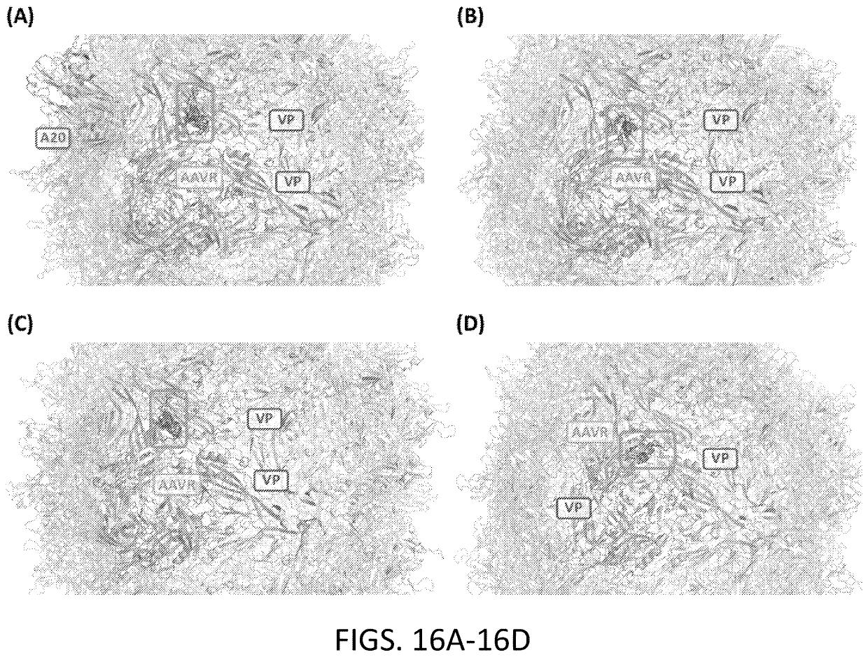

Similar results of transgene enrichment (2-5-fold), yield (76-100%), and HCP removal (LRV ~2.3-2.7) were obtained with the AAV bound in acidic environment (FIGS. 16A-16D). Once again, most of the bound capsids were eluted in 0.4 M MgCl₂ at pH 6.8, with the sole exception of AAV5, for which the q/ddPCR and transduction assays failed (data not shown but can be made available upon request).

### Example 8

Development of alkaline-stable variants of ligand A10. The lifetime of chromatographic adsorbents for AAV purification is currently a lesser concern than purification performance, given the small cohorts of patients receiving AAV-based drugs. However, several efforts are underway to increase the effective transgene size (e.g., by deleting sequences not involved in the protein's function, leveraging the ability of vector genomes to “concatemerize” upon cell entry, or using multiple vectors to deliver overlapping fragments of a larger gene) thus expanding the range of therapeutic indications enabled by AAVs. Should these innovation come to fruition, it is reasonable to expect that the need of reusing affinity resins—and the corresponding cGMP practices—currently in force for blockbuster biotherapeutics (e.g., monoclonal antibodies) will expand to the chromatographic purification of viral vectors. In this context, the ability of affinity ligands to withstand caustic cleaning-in-place (CIP) is paramount to ensure safe reusability.

Ligand A10 contains a glutamin (Q), which is rapidly deamidated to glutamic acid (E) in alkaline conditions, resulting in a loss of AAV affinity. To prevent this issue, variants CVIDGSASTDDDRIC (A10′; SEQ ID NO: 33), CVIDGSSSTDDDRIC (A10″; SEQ ID NO: 34), and CVIDGSASTDDDHIC (A10′″; SEQ ID NO: 35) were designed by replacing Q with either an alanine (A) or a serine(S) residue. These substitutions, however, modify the folding and therefore the binding mechanism of A10, making it necessary to replace the original lysine residue (K) with a cationic amino acid capable of forming more hydrogen bonds, such as arginine (R) or histidine (H). The serotype-agnostic binding activity of A10′, A10″, and A10′″ was first evaluated in silico via molecular docking and dynamic studies, which confirmed the identity of the binding footprint and energy of variant A10′ and its precursor A10 (FIGS. 16A-16D).

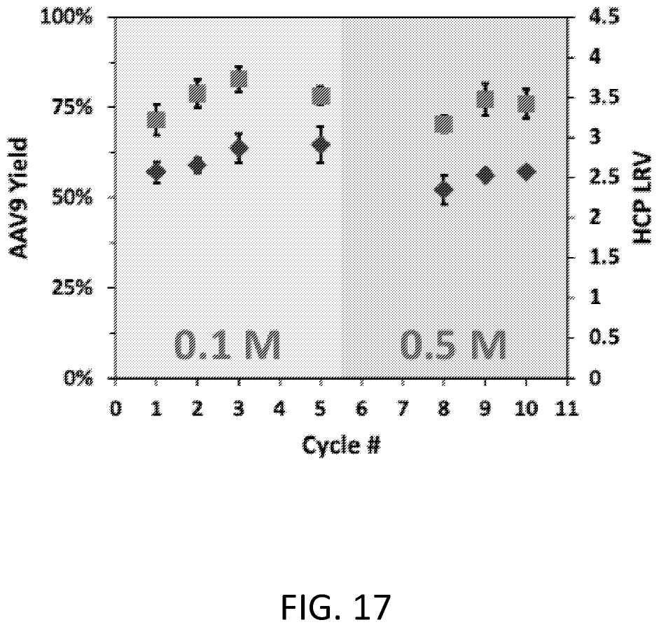

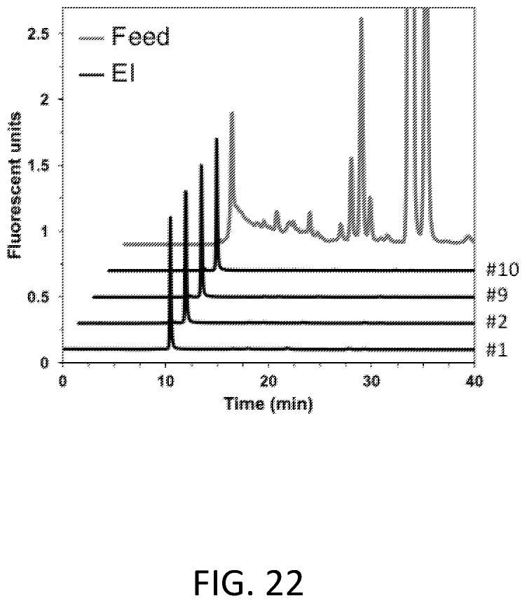

Experiments were therefore conducted to test the ability of A10′-Toyopearl resin to perform 10 consecutive cycles of AAV9 purification with intermediate CIP using 0.1 or 0.5 M NaOH (15 CVs in flow followed by 30 min of static contact). The values of capsid yield and HCP clearance, summarized in FIG. 17 and the SEC analysis of the eluates obtained at successive cycles (FIG. 22) demonstrate the purification ability and stability of A10′, positioning it as an excellent candidate for large-scale manufacturing of therapeutic AAVs.

What is claimed is:

1. A composition for purifying an adeno-associated virus (AAV) from a biological fluid, wherein the composition comprises at least one peptide ligand that is at least ten amino acids in length and comprises an AAV-binding motif having at least 80% identity to one of the following:
(a)
(SEQ ID NO: 18)
YIHFSGYT;
(b)
(SEQ ID NO: 19)
STDDD;
(c)
(SEQ ID NO: 20)
CYHFS;
and/or
(d)
(SEQ ID NO: 21)
LITHPRDYS.

2. The composition of claim 1, wherein the AAV is a recombinant AAV (rAAV).

3. The composition of claim 1 or claim 2, wherein the at least one peptide ligand is cyclic.

4. The composition of any one of claims 1 to 3, wherein the AAV is selected from the group consisting of AAV1, AAV2, AAV3, AAV4, AAV5, AAV6, AAV7, AAV8, AAV9, and AAVrh10.

5. The composition of claim 4, wherein the AAV is selected from the group consisting of AAV1, AAV2, AAV5, AAV6, AAV8, and AAV9.

6. The composition of any one of claims 1 to 5, wherein the at least one peptide ligand comprises an AAV-binding motif having at least 80% identity with SEQ ID NO: 18, and further comprises an amino acid sequence having at least 80% identity with any one of SEQ ID NOs: 1, 2, 6, 7, and 14-16.

7. The composition of any one of claims 1 to 5, wherein the at least one peptide ligand comprises an AAV-binding motif having at least 80% identity with SEQ ID NO: 19, and further comprises an amino acid sequence having at least 80% identity with any one of SEQ ID NOs: 10-13, and 33-35.

8. The composition of any one of claims 1 to 5, wherein the at least one peptide ligand comprises an AAV-binding motif having at least 80% identity with SEQ ID NO: 20, and further comprises an amino acid sequence having at least 80% identity with any one of SEQ ID NOs: 3-5, 8, and 9.

9. The composition of any one of claims 1 to 5, wherein the at least one peptide ligand comprises an AAV-binding motif having at least 80% identity with SEQ ID NO: 21, and further comprises an amino acid sequence having at least 80% identity with any one of SEQ ID NO: 17.

10. The composition of any one of claims 1 to 9, wherein the at least one peptide ligand binds AAV1 by interacting with at least one of the following amino acids located on the solvent-accessible surface of the conserved regions of its capsid virion proteins (VP1, VP2, and VP3): THR504, ASN500, SER499, TRP503, ASN269, ASP270, SER268, ASN271, ALA267, GLY266, HIS272, SER262, SER385, ALA263, GLN386, GLY384, ASN383, ASN512, GLY513, and/or LYS508.

11. The composition of any one of claims 1 to 9, wherein the at least one peptide ligand binds AAV2 by interacting with at least one of the following amino acids located on the solvent-accessible surface of the conserved regions of its capsid virion proteins (VP1, VP2, and VP3): GLN385, THR503, GLU499, TRP502, LYS507, ASN268, ASP269, SER267, ALA266, GLY265, HIS271, SER384, SER264, GLN263, SER262, GLY383, ASN382, and/or ASN511.

12. The composition of any one of claims 1 to 9, wherein the at least one peptide ligand binds AAV5 by interacting with at least one of the following amino acids located on the solvent-accessible surface of the conserved regions of its capsid virion proteins (VP1, VP2, and VP3): SER319, GLU708, GLN532, PRO533, ASN535, TYR542, ALA534, ASN530, ASN546, ASP704, SER531, GLY545, ARG710, PHE698, MET547, THR711, GLU544, THR712, LEU548, ARG713, GLN697, LEU543, THR541, ALA540, THR538, and/or GLY478.

13. The composition of any one of claims 1 to 9, wherein the at least one peptide ligand binds AAV9 by interacting with at least one of the following amino acids located on the solvent-accessible surface of the conserved regions of its capsid virion proteins (VP1, VP2, and VP3): GLY266, ASN262, THR264, SER263, GLY267, GLU500, SER499, ASN498, PRO504, TRP503, SER269, ASN270, ASP271, SER268, SER386, ALA273, GLN387, ASP384, and/or GLY385.

14. The composition of any one of claims 1 to 9, wherein the at least one peptide ligand binds AAV2 by interacting with at least one of the following amino acids located on the solvent-accessible surface of the conserved regions of its capsid virion proteins (VP1, VP2, and VP3): THR716, ASN717, VAL719, VAL708, LYS706, ASN709, GLU548, LYS556, and/or ASP553.

15. The composition of any one of claims 1 to 14, wherein the at least one peptide ligand comprises more than one of the AAV-binding motifs of SEQ ID NOs: 18-21.

16. The composition of any one of claims 1 to 15, wherein the at least one peptide ligand comprises any combination of the AAV-binding motifs of SEQ ID NOs: 18-21.

17. The composition of any one of claims 1 to 16, wherein the at least one peptide ligand is no more than 25 amino acids in length.

18. The composition of any one of claims 1 to 17, wherein the at least one peptide ligand comprises an isoelectric point from about 3.5 to about 9.5.

19. The composition of any one of claims 1 to 18, wherein the at least one peptide ligand comprises a polarity value from about-1.2 to about 1.2.

20. The composition of any one of claims 1 to 19, wherein the at least one peptide ligand exhibits a disassociation constant (K_{D}) less than or equal to about 10⁻⁵ M at a pH that is higher than or equal to 7.0.

21. The composition of any one of claims 1 to 20, wherein the at least one peptide ligand exhibits a disassociation constant (K_{D}) higher than or equal to about 10⁻⁴ M at a pH that is lower than or equal to 6.5.

22. The composition of any one of claims 1 to 21, wherein the at least one peptide ligand from (a) and/or (b) exhibits a dynamic binding capacity (DBC_{10%}) of at least 10¹³ vp/mL of resin.

23. The composition of any one of claims 1 to 22, wherein the at least one peptide ligand from (a) comprises:
(i) an amino acid sequence having at least 80% sequence identity with SEQ ID NO: 1;
(ii) an amino acid sequence having at least 80% sequence identity with SEQ ID NO: 2;
(iii) an amino acid sequence having at least 80% sequence identity with SEQ ID NO: 3;
(iv) an amino acid sequence having at least 80% sequence identity with SEQ ID NO: 4;
(v) an amino acid sequence having at least 80% sequence identity with SEQ ID NO: 5;
(vi) an amino acid sequence having at least 80% sequence identity with SEQ ID NO: 6;
(vii) an amino acid sequence having at least 80% sequence identity with SEQ ID NO: 7;
(viii) an amino acid sequence having at least 80% sequence identity with SEQ ID NO: 8;
(ix) an amino acid sequence having at least 80% sequence identity with SEQ ID NO: 9;
(x) an amino acid sequence having at least 80% sequence identity with SEQ ID NO: 10;
(xi) an amino acid sequence having at least 80% sequence identity with SEQ ID NO: 11;
(xii) an amino acid sequence having at least 80% sequence identity with SEQ ID NO: 12;
(xiii) an amino acid sequence having at least 80% sequence identity with SEQ ID NO: 13;
(xiv) an amino acid sequence having at least 80% sequence identity with SEQ ID NO: 14;
(xv) an amino acid sequence having at least 80% sequence identity with SEQ ID NO: 15;
(xvi) an amino acid sequence having at least 80% sequence identity with SEQ ID NO: 16;
(xvii) an amino acid sequence having at least 80% sequence identity with SEQ ID NO: 17;
(xviii) an amino acid sequence having at least 80% sequence identity with SEQ ID NO: 33;
(xix) an amino acid sequence having at least 80% sequence identity with SEQ ID NO: 34; and/or
(xx) an amino acid sequence having at least 80% sequence identity with SEQ ID NO: 35.

24. The composition of any one of claims 1 to 23, wherein the composition comprises:
(i) at least two peptide ligands from SEQ ID NOs: 1-17 and 33-35;
(ii) at least three peptide ligands from SEQ ID NOs: 1-17 and 33-35;
(iii) at least four peptide ligands from SEQ ID NOs: 1-17 and 33-35;
(iv) at least five peptide ligands from SEQ ID NOs: 1-17 and 33-35;
(v) at least six peptide ligands from SEQ ID NOs: 1-17 and 33-35;
(vi) at least seven peptide ligands from SEQ ID NOs: 1-17 and 33-35;
(vii) at least eight peptide ligands from SEQ ID NOs: 1-17 and 33-35;
(viii) at least nine peptide ligands from SEQ ID NOs: 1-17 and 33-35;
(ix) at least ten peptide ligands from SEQ ID NOs: 1-17 and 33-35;
(x) at least eleven peptide ligands from SEQ ID NOs: 1-17 and 33-35;
(xi) at least twelve peptide ligands from SEQ ID NOs: 1-17 and 33-35;
(xii) at least thirteen peptide ligands from SEQ ID NOs: 1-17 and 33-35;
(xiii) at least fourteen peptide ligands from SEQ ID NOs: 1-17 and 33-35;
(xiv) at least fifteen peptide ligands from SEQ ID NOs: 1-17 and 33-35;
(xv) at least sixteen peptide ligands from SEQ ID NOs: 1-17 and 33-35;
(xvi) at least seventeen peptide ligands from SEQ ID NOs: 1-17 and 33-35;
(xvii) at least eighteen peptide ligands from SEQ ID NOs: 1-17 and 33-35;
(xviii) at least nineteen peptide ligands from SEQ ID NOs: 1-17 and 33-35; or
(xix) all twenty peptide ligands of SEQ ID NOs: 1-17 and 33-35.

25. The composition of any one of claims 1 to 24, wherein the at least one peptide ligand comprises a linker.

26. The composition of any one of claims 1 to 25, wherein the at least one peptide ligand is bound to a solid support.

27. The composition of claim 26, wherein the solid support comprises a non-porous or porous particle, a porous monolith, a cast membrane, a plastic surface, a fiber or a woven or non-woven fibermat, a hydrogel, a microplate, and/or a microfluidic device.

28. The composition of claim 27, wherein the solid support comprises polymethacrylate, polyolefin, polyether, polyester, crosslinked polysaccharide, iron oxide, silica, titania, and/or zirconia.

29. The composition of any one of claims 1 to 22, wherein the biological fluid is a cell culture fluid.

30. The composition of any one of claims 1 to 29, wherein the biological fluid comprises a supernatant and/or a cellular lysate.

31. The composition of claim 29 or claim 30, wherein the biological fluid is derived from a virus production cell line.

32. The composition of claim 31, wherein the virus production cell line is selected from the group consisting of CHO cells, HEK293 cells, MDCK-S, MDCK-A, Vero cells, LLC-MK2D, PER.C6, EB66, AGE1.CR cells, Spodoptera frugiperda (Sf9) cells, and HeLa cells, or any derivatives or variants thereof.

33. An adsorbent comprising the composition of any of claims 1 to 32.

34. A method of purifying an adeno-associated virus (AAV) from a biological fluid, the method comprising:
contacting the composition comprising the at least one peptide ligand of any one of claims 1 to 32, or the adsorbent of claim 33, with a biological fluid comprising the AAV, wherein the at least one peptide ligand binds the AAV; and
eluting the AAV from the at least one peptide ligand.

35. The method of claim 34, wherein the elution is performed at pH from about 6.0 to about 7.5.

36. The method of claim 34 or claim 35, wherein the composition comprising the at least one peptide ligand of any one of claims 1 to 32, or the adsorbent of claim 33, is substantially resistant to NaOH, or a similar alkaline agent, at a concentration from about 0.1M to about 0.5M.

37. The method of any one of claims 34 to 36, wherein the composition comprising the at least one peptide ligand of any one of claims 1 to 32, or the adsorbent of claim 33, is substantially reusable for up to about 10 cycles.

38. The method of any one of claims 34 to 37, wherein the method further comprises a washing step before eluting the AAV from the at least one peptide ligand.

39. The method of any one of claims 34 to 38, wherein the method results in at least a 50% yield for the AAV.

40. The method of any one of claims 34 to 39, wherein the method produces at least an 80-fold reduction in host cell proteins.

41. An adeno-associated virus (AAV) purified using the method of claim 34, wherein the AAV exhibits at least 25% transduction efficiency.
